(19) United States
(12) Patent Application Publication
(10) Pub. No.: US 2026/0256980 A1
(43) Pub. Date: Sep. 3, 2026

(54) MEDICINAL ADHESIVE COMPOSITIONS

(71) Applicant: THE REGENTS OF THE UNIVERSITY OF CALIFORNIA, Oakland, CA (US)

(72) Inventors: PHILLIP B. MESSERSMITH, Berkeley, CA (US); SUBHAJIT PAL, Berkeley, CA (US)

(21) Appl. No.: 19/067,344

(22) Filed: Feb. 28, 2025

(51) Int. Cl. A61L 24/04

(52) U.S. Cl. CPC A61L 24/046; A61L 2430/06; A61L 2430/10; A61L 2430/34

(57) ABSTRACT

Medicinal adhesive compositions and methods of use are provided. These can include cyclic disulfide monomers, oligomers of reactions between the cyclic disulfides, polymers of the cyclic disulfides, solid articles formed from the polymers, and any combination thereof. The compositions can be adhesive, and can also deliver drugs. Methods of making and using the compositions are provided, including use of the compositions on a variety of substrates that include biological tissue, biomaterials, and various substrates including synthetics and natural such as bone, wood, and cellulosics; metals and alloys; polymers, plastics, and rubbers; ceramics; composites; and combination thereof. The adhesives can adhere materials in a variety of environmental conditions including ambient conditions, wet conditions including underwater, and in a variety of temperatures and pressures found in medical environments, including in vitro and living tissue environments. Any one or combination of bioactive agents can be delivered to a target tissue by these compositions.

### CROSS-REFERENCE TO RELATED APPLICATIONS

This application does not claim priority to any other applications.

### BACKGROUND

### Field of the Invention

The systems and methods taught herein are generally directed to medicinal adhesive compositions.

### Description of the Related Art

The technology provided herein includes a medicinal adhesive composition and methods of using the composition. Poor water solubility is known to affect up to 40% of potentially valuable drug candidates, and such drugs administered through implants are better absorbed, requiring lower dosages and improving bioavailability. Moreover, another advantage of drug-eluting implants is patient compliance. An implant can be designed, for example, to release medication at a desired rate, overcoming intentional and unintentional non-compliance, either of which can advance disease, increase healthcare costs, and increase risk of death. In addition, implants can release drug in a targeted area, allowing for use of a reduced dosage that can obtain a more targeted effect. For at least these reasons, the skilled artisan will appreciate the technology provided herein.

It should be appreciated that there is plenty of room for technical innovations in the development of medical adhesives, particularly for specialized medical uses. Rapid curing and high mechanical strength are certainly desired features of many adhesives, but a longfelt-but-unsolved need is an adhesive that not only cures rapidly with a high strength, but one that also adheres well to a wet surface. Those of skill will understand that many desirable adhesives are manmade and, unfortunately for the medical field, it's unusual for a manmade adhesive to adhere well to a wet surface. An example of state-of-the-art medical adhesives are the cyanoacrylates. These “superglues” are used, for example, in external wound closures due to their rapid cure, high strength, and good infection barrier properties. Unfortunately, while the cyanoacrylates are approved and widely used for external wound closure, they're not approved by the Food and Drug

Administration (FDA) for many internal uses because of their high cytotoxicity and inability to degrade and excrete from the body. Hydrogel-based adhesives, such as fibrin glue, albumin glue, and polyethylene glycol (PEG)-based glues, have been approved by the FDA for internal procedures but, unfortunately, these suffer from poor mechanical strength, often at least an order of magnitude weaker than the cyanoacrylates. The market for medical adhesives is highly segmented, the superglues used primarily for external wound closure, and hydrogel adhesives used for internal adhesion and sealing. As such, those of skill know that the art suffers a longfelt-but-unsolved need for an adhesive that can do both, serve as an adhesive and sealant for both external and internal medical uses. As such, one market segment that can be improved is the creation of a medical adhesive that works for both internal and external applications, has rapid curing and a high mechanical strength, can adhere to wet surfaces, can degrade and excrete from the body, and has an acceptable cytotoxicity on degradation.

Another market segment that can be improved by the adhesives taught herein is the PSA medical adhesive market. Pressure sensitive adhesives (PSAs) are examples of topical adhesive products that are valuable in the medical sector. PSAs are used, for example, in wound dressings, such as wound coverings that cover wounds and promote healing, and dressing tapes to secure the dressings to skin. PSAs are also used to attach surgical drapes to a patient while protecting an incision site. PSAs are also used to attach wearable devices to the skin of a subject, including, for example, insulin pumps, EEG sensors, pulse oximetry devices, and location sensors for dementia patients. Moreover, PSAs can help to ease assembly of medical devices.

Another market segment that can be improved by the adhesives taught herein are hemostats and sealants, products that are valuable in the medical sector. Hemostats adhere to damaged tissue and seal injured vessels. Hemostats can concentrate clotting factors, absorbing water from blood to concentrate clotting factors in a wound, and they can activate coagulation pathways to promote clot formation. Surgical sealants are commonly used to prevent leakage of fluid and/or gas from a surgical incision to repair tissue damaged by injury or the surgery itself. Sealants are commonly used on skin, internal organs and blood vessels to help reduce blood loss or prevent leaks, often in combination with sutures and staples. One of skill will appreciate that state-of-the-art hemostats can suffer from infection, inflammation, and foreign body reactions; and, state-of-the-art sealants can suffer from toxic degradation effects, intense inflammatory response, and risk of cancer. The adhesive compositions herein can address these issues and, with the addition of drug for delivery at a targeted site, can address these issues with the release of one or more active agents.

Recently, poly(α-lipoic acid) has attracted significant attention due to its excellent mechanical and adhesion strength, along with cost-effective production and close-loop chemical recycling. α-lipoic acid (L1), also known as thioctic acid, is a five-member cyclic disulfide with a carboxylic acid side chain. It is an essential cofactor for aerobic metabolism in animals. The (R)-form of α-lipoic acid is found in nature, but a racemic mixture of α-lipoic acid is also considered safe and has been widely used as a dietary supplement. α-lipoic acid is mainly used as an antioxidant due to its exceptional ability to quench reactive oxygen species. Besides having a favorable biological profile, α-lipoic acid also shows exceptional potential for ring-opening polymerization due to the release of ring strain from the polymerization. In 1956, R. C. Thomas and L. J. Reed first observed that α-lipoic acid undergoes polymerization thermally, when heated above its melting temperature. Unfortunately, the polymer had poor stability at ambient conditions due to spontaneous depolymerization. Since then, multiple attempts have been made to synthesize a stable form poly(α-lipoic acid).

Asparagusic acid is another example. Although asparagusic acid is known to exist in nature, isolation of asparagusic acid on a larger scale in its pure monomeric form is challenging. Because of this, asparagusic acid can be synthesized in the laboratory or purchased at a high cost, and consists of a mixture of monomers and oligomer (<10% oligomer). And, moreover, poly(asparagusic acid) does not exist in nature, and has not been produced in the art. As such, poly(asparagusic acid) is novel and inventive as a composition in itself. Asparagusic acid (1,2-dithiolane-4-carboxylic acid) is unique to asparagus and is of great interest as a monomer, namely due to research of pharmacological properties of the monomers that include beneficial biological activity in the treatment of urinary problems, fertility, breast milk production, and diseases that include kidney, bladder, rheumatic, liver disease, asthma and cancer.

In discovering how to stabilize these polymers, it was discovered that the rate of biodegradation can be selected through the choice of stabilizer and/or terminator moieties. This provides a design feature for adjusting the biodegradation rate of the polymer for a particular product or use. The biodegradation rate can be adjusted by first deciding whether a stabilizer and/or terminator moiety is needed at all for a desired use, and then by choosing a stabilizer and/or terminator for the bond strengths provided with the polymer, where the choice of bond strength can facilitate the selection and design of a polymer for a desirable biodegradation rate.

As described above, the adhesives taught herein have inherent medicinal qualities that are a result of the biodegradation and liberation of monomers during biodegradation, the monomers themselves having desired biologic activities. The medicinal qualities of the polymers can be enhanced and tailored for a particular application by adding additional active agents for particular products and uses. The medical adhesives provided herein, for example, can be configured to have any one, or any set of, desired therapeutic activities that include, but are not limited to, antimicrobial activity, anti-inflammatory activity, antiviral activity, antivirulent activity, chemotherapeutic activity, immunomodulatory activity, hemostat activity, and the like.

Those of skill in the art of medical adhesives would appreciate at least the following: biocompatible and biodegradable adhesive compositions that (i) work for both internal and external applications, (ii) cure rapidly, (iii) have high mechanical strength, (iv) adhere to wet surfaces, (v) degrade at an adjustable rate, (vi) excrete from the body, (vii) have an acceptable cytotoxicity on degradation, the monomers have desired medicinal activities, and the adhesives (viii) can be further configured to have any one, or any set of, desired therapeutic activities, including delivery of drugs attached to the polymer mechanically, ionically, covalently, by hydrogen bonding, or some combination thereof, for example.

### SUMMARY

Stable medical compositions for medical uses are provided and can include cyclic disulfide monomers, oligomers of reactions between the cyclic disulfides, polymers of the cyclic disulfides, solid articles formed from the polymers, and any combination thereof. The compositions can be adhesive, binding tissue-to-substrate, tissue-to-tissue, substrate-to-substrate, or a coating for a substrate. Methods of making and using the compositions are provided, including use of the compositions on a variety of substrates that include biological tissue, biomaterials, and various substrates including synthetics and natural such as bone, wood, and cellulosics; metals and alloys; polymers, plastics, and rubbers; ceramics; composites; and combination thereof. The adhesives can adhere in a variety of environmental conditions including ambient atmospheric conditions, wet conditions including underwater, and in a variety of temperatures and pressures found in medical environments, including in vitro and living tissue environments. Bioactive agents can be delivered to a target tissue by the adhesive compositions, allowing for the design of many different types of medicinal compositions, including medicinal adhesive compositions that can be applied to a variety of medical products and uses, both topical and internal, including surgical uses.

Medical products are provided herein. In some embodiments, the medical products comprising:
a composition having a liquid, powder, solid, or a combination thereof, and the composition including
   a plurality of substituted 1,2-dithiolane monomers, the substituted 1,2-dithiolane monomers functional to polymerize through a ring-opening reaction to form a polydisulfide polymer having
   a first active thiol end and a second active thiol end; and,
   a plurality of repeating units having a substituted dithioalkyl structure from the ring opening reaction as follows

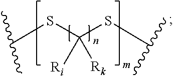

In some embodiments,
n is 3;
each Rᵢ and Rₖ is independently selected from the group consisting of H; alkyl, cycloalkyl, alkenyl, alkynyl, and aryl groups, each of the groups having from 1-8 carbons; hydroxylated alkyl, cycloalkyl, alkenyl, alkynyl, and aryl groups, each of the groups having from 1-8 carbons; and, carboxylated alkyl, cycloalkyl, alkenyl, alkynyl, and aryl groups, each of the groups having from 1-8 carbons; wherein, i and k are integers, and at least one Rᵢ or Rₖ in each repeating unit includes a carbonyl functionality;
and,
m is an integer selected to match a desired molecular weight of the polymer.

In some embodiments, a plurality of stabilizer molecules can be configured to stabilize the first active thiol end of the polymer, the plurality of stabilizer molecules being the substituted cyclic disulfide molecules derivatized to include a functional group that forms a first labile bond with the first active thiol end, the first labile bond selected from the group consisting of a thioester bond, a thiocarbamate bond, and a thioether bond.

Moreover, in some embodiments, the compositions can include a bioactive agent.

In some embodiments, the polydisulfide polymer can
be formed by reacting the plurality of monomers and the plurality of stabilizers in the composition;
adhere to a biological tissue in water;
be biocompatible and biodegradable.

In some embodiments, the polydisulfide polymer can be designed to release the bioactive agent for delivery to a target tissue.

In some embodiments, the composition further including a plurality of terminator molecules for forming a labile bond with the second active thiol group, the plurality of terminator molecules selected from the group consisting of R′OH, R′CO₂H, and R′SH, and R′ is selected from the group consisting of alkyl, cycloalkyl, alkenyl, cycloalkenyl, alkynyl, cycloalkynyl, and aryl groups, each of the groups having from 1-8 carbons; and, the second labile bond is selected from the group consisting of thioethers, thioesters, and disulfides.

In some embodiments, the composition further including a plurality of terminator molecules for forming a labile bond with the second active thiol group, the plurality of terminator molecules selected from the group consisting of alkanols having from 1-8 carbons, alkanoic acids having from 1-8 carbons, and alkylthiols having from 1-8 carbons, and the second labile bond is selected from the group consisting of thioethers, thioesters, and disulfide.

In some embodiments, the composition further including a plurality of terminator molecules for forming a labile bond with the second active thiol group, the plurality of terminator molecules selected from the group consisting of ethanol, and a substituted 1,2-dithiolane with a hydroxyl functionality.

In some embodiments, the repeating unit is a substituted 1,3-dithiopropyl structure as follows

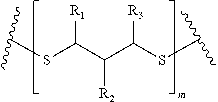

wherein,
m is an integer;
Rᵢ includes R₁, R₂, and R₃; and,
each Rₖ is H;
and,
each stabilizer molecule includes the substituted 1,3-dithiopropyl structure.

In some embodiments, the repeating unit is a substituted 1,3-dithiopropyl structure as follows

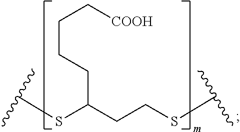

wherein,
m is an integer;
R₁ is a pentanoic acid group, and R₂ and R₃ are each H;
and,
each stabilizer molecule includes the substituted 1,3-dithiopropyl structure, wherein R₁ is a pentanoic acid group, and R₂ and R₃ are each H;
wherein, the bioactive agent is releasably attached chemically to a carboxylate group in at least one of the repeating units;
   chemically to a sulfur in at least one of the repeating units;
   chemically to the first active thiol end and/or the second active thiol end;
   mechanically to the polymer;
   or a combination thereof.

In some embodiments, the bioactive agent is a wound healing agent.

Methods of treatment are also include. In some embodiments, a method of adhering a first biological tissue to a substrate, the method comprising:
applying the medical product of claim 1 to the first biological tissue; and,
creating a joint between the first tissue and the substrate to adhere the first biological tissue to the substrate.

In some embodiments, the substrate is a second biological tissue, and the second biological tissue is selected from the group consisting of a lung tissue, a stomach tissue, a bladder tissue, an intestinal tissue, a vascular tissue, a heart tissue, a dural tissue, an ocular tissue, a cartilage tissue, skin tissue, a fetal membrane tissue, and a tendon tissue.

In some embodiments, the substrate is a medical device selected from the group consisting of wound dressings, collagen membranes, and ostomy devices.

In some embodiments, the method is a wound healing treatment of a subject, the bioactive agent selected to include a wound healing agent.

In some embodiments, the method is a topical tissue closure treatment of a subject, the bioactive agent selected to assist in the topical tissue closure.

In some embodiments, the method is a skin graft treatment of a subject, the bioactive agent selected to assist in the skin graft.

In some embodiments, the method is a wound dressing attachment in a treatment of a subject, the bioactive agent selected to include a wound healing agent.

In some embodiments, the method is a medical device attachment in a treatment of a subject, the bioactive agent selected to assist in the treatment of the subject.

In some embodiments, the method is an internal tissue closure treatment of a subject, the bioactive agent selected to assist in the internal tissue closure.

In some embodiments, the method is an internal tissue closure treatment of a subject, and the internal closure treats an organ puncture, the bioactive agent selected to assist in the internal tissue closure of the organ puncture.

In some embodiments, the method is an internal cartilage repair treatment of a subject, the bioactive agent selected to assist in the internal cartilage repair.

In some embodiments, the method is an internal anastomosis repair treatment of a subject, the bioactive agent selected to assist in the internal anastomosis repair.

In some embodiments, the method is an internal tendon or ligament repair treatment of a subject, the bioactive agent selected to assist in the tendon repair, ligament repair, or a combination thereof.

There are several methods of using the adhesive compositions in their various forms, and the adhesive compositions can be used in a variety of conditions. In some embodiments, the adhesives are used in ambient conditions. In some embodiments, the adhesives are used in dry conditions. In some embodiments, the adhesives are used in wet conditions. In some embodiments, the adhesives are used underwater.

There are a variety of products that can be made from the adhesive compositions, and the products can be liquid, powder, solid, or a combination of thereof. In some embodiments, the adhesive compositions include monomers that are precursors for polymerization to adhesive polymers. In some embodiments, the adhesive compositions include monomers and oligomers that are precursors for polymerization to adhesive polymers. In some embodiments, the adhesive compositions include monomers oligomers that are precursors for polymerization to adhesive polymers, and polymers that are the product of partial polymerization of the monomers, or monomers and oligomers. In some embodiments, adhesive compositions can be a combination of monomers, oligomers, and polymers, alone or in any combination. The adhesive composition products can also include any one or any combination of a component selected from the group consisting of a stabilizer, a terminator, the conjugate bases of the monomers, DMSO, an acid such as a Lewis acid or Bronsted acid, a cross-linker, and a reaction solvent. And, of course, any of the adhesive compositions can be further configured to have any one, or any set of, desired therapeutic activities, including delivery of drugs attached to the polymer mechanically, ionically, covalently, by hydrogen bonding, or some combination thereof, for example . . .

The stability of the polymers in the adhesive compositions can be adjustable, in some embodiments, providing control over the rate of biodegradation of the polymer. In some embodiments, the biodegradation rate of the polymer can be used to control the rate of release of the bioactive agent. The choice of stabilizer moieties further allows to design the stability of the polymer, by choosing whether the stabilizer is needed at all for a desired use, and then adjusting the stability of the polymer for it's desired use by choosing a stabilizer moiety having a desired bond strength between the stabilizer and polymer. The same is true of the choice of terminator moieties by choosing whether to the terminator moiety is needed at all for the desired use, and then further adjusting the stability of the polymer choosing a terminator moiety having a desired bond strength between the terminator and polymer. Moreover, the bioactive agent can be included with a selection of stabilizer and/or terminator.

The biocompatible and biodegradable adhesive compositions can be used for any of a variety of medical uses. In some embodiments, the adhesive compositions can be used as a medical tissue adhesive, a medical tissue sealant, a medical tissue hemostat, a medical device fastener, a drug delivery device and combinations thereof.

### BRIEF DESCRIPTION OF THE FIGURES

FIG. 1A illustrates examples of dithiolane monomers and stabilizers that can be used in the adhesive compositions, namely those made from α-lipoic acid and asparagusic acid, in some embodiments.

FIG. 1B illustrates the conversion of a liquid adhesive composition to a polydisulfide polymer, in some embodiments. The liquid adhesive contains α-lipoic acid monomer (L1), an NHS-derivatized α-lipoic acid (L2) as a first stabilizer, and a Gly-OSu-derivatized α-lipoic acid (L3) as a second stabilizer. The liquid adhesive composition can sometimes be referred to as a “precursor” solution because it is a composition that can form the poly(α-lipoic acid) polymer when water, a polar, protic solvent, is added to the composition. The polymer has a repeating disulfide unit as shown, with labile thioester bonds in the polymer.

FIGS. 1C1, 1C2, and 1C3 illustrate a solid adhesive composition of solid monomers and stabilizer(s), a liquid adhesive composition of a solution of the monomers and stabilizer(s) in a reaction solvent, and an adhesive composition in which the monomers and stabilizer(s) have been polymerized in an aqueous polymerization, in some embodiments. FIG. 1C1 illustrates a solid mixture of α-lipoic acid monomer (L1), an NHS-derivatized α-lipoic acid (L2) as a first stabilizer, and a Gly-OSu-derivatized α-lipoic acid (L3) as a second stabilizer. FIG. 1C2 illustrates the solid mixture in solution that has not yet polymerized and, as such, can be referred to as a “precursor” solution which can be obtained by dissolving the solid composition of FIG. 1C1 in pure ethanol, for example. FIG. 1C3 is the polymerized version of the either FIG. 1C1 or FIG. 1C2 that can be obtained by adding a polar protic solvent such as water, an aqueous solution of water and ethanol, perhaps along with DMSO and/or acid as a polymerization catalyst.

FIGS. 1D1, 1D2, and 1D3 illustrate a liquid adhesive composition containing polymers with monomers, the conjugate base of the monomers, and stabilizer(s); brushing of the liquid adhesive composition onto a substrate, and aqueous polymerization of the liquid adhesive composition on the substrate to create a sticky adhesive on the substrate, in some embodiments. FIG. 1D1 shows a liquid adhesive composition of α-lipoic acid monomer (L1), an NHS-derivatized α-lipoic acid (L2) as a first stabilizer, and the conjugate base of the α-lipoic acid monomer (L4) to help stabilize the “precursor” from an undesired polymerization. The precursor can be brushed onto a substrate which can be applied to a wet surface to polymerize as shown in FIG. 1D2, the aqueous polymerization that results from contact with a wet surface is shown in FIG. 1D3.

FIG. 1E illustrates the stepwise conversion of a liquid adhesive composition to a wet polymer that can be applied to a substrate, or be formed into a solid shape before or after polymerization, to create a solid article such as a solid adhesive patch, in some embodiments. The unpolymerized liquid adhesive composition is shown to polymerize to a wet polymer when water is added to the liquid adhesive “precursor” composition, and the wet polymer can be used to form the solid adhesive patch.

FIG. 1F illustrates the stepwise conversion of a liquid adhesive composition to a wet polymer that can be applied to a substrate as a pressure sensitive adhesive (PSA), in some embodiments. The unpolymerized liquid adhesive composition is polymerized, and the sticky polymer adhesive is added to a substrate as a PSA. A paper substrate with a strip of the PSA, for example, can be a sticky note in some embodiments.

FIG. 2A measures the stress-strain curve of the synthesized polymer at different strain rates, 200 mm/min, 400 mm/min, and 600 mm/min, in some embodiments. The results show that the polymer can be stretched over 10 times of its initial length (1000% strain deformation) without rupture.

FIG. 2B is a cyclic stress-strain measurement with subsequent incremental strain without intermediate resting, in some embodiments. The polymer shows excellent recovery at the lower deformation region, whereas another cyclic stress-strain measurement with subsequent incremental strain without intermediate resting indicates excellent fatigue resistance of the obtained polymer.

FIG. 2C is a stress-strain curve taken after 2 min healing at 37° C., in some embodiments. A polymer film was cut into two pieces and then carefully brought into contact for 2 min at 37° C. and equilibrated for 5 min at room temperature before measurements were taken. Interestingly, and surprisingly, FIG. 2C illustrates that over 60% recovery efficiency was achieved within such a short interval, indicating an unexpectedly high chain mobility within the polymer network.

FIGS. 2D and 2E compares the storage modulus (squares) and the loss modulus (triangles) between solid patches, in-situ synthesized polymer after 5 minutes incubation in PBS at 37° C., and in-situ synthesized polymer after 24 hours incubation in PBS at 37° C., in some embodiments. FIG. 2D is a frequency sweep measurement at a constant strain of 0.4% at 37° C., and FIG. 2E is an amplitude sweep at a constant frequency of 5 rad/s at a temperature of 37° C., in some embodiments. Without intending to be bound by any theory or mechanism of action, the data suggests that the polymer network has a lower crosslinking density, and it increases over time and provides higher stability of the network.

FIG. 3A illustrates a comparison of the lap shear strengths of the adhesive polymer on wet bovine pericardium and pig skin at different concentrations (7 mol % and 14 mol %) of the stabilizer (L2) referred to as the N-hydroxysuccinimide (NHS)-derivative of the α-lipoic acid monomer, in some embodiments. The p value was calculated with a student's t-test in Microsoft Excel (2 array, 2 tails, 2 type). * p≥0.05, ** p≤0.05, *** p≤0.01. No significant difference in the shear strength was observed at the different stabilizer concentrations.

FIG. 3B illustrates a comparison of the lap shear strengths of the adhesive polymer on wet bovine pericardium when using a different stabilizer (L3) that is a second derivatized α-lipoic acid monomer referred to as the Gly-OSu derivative of α-lipoic acid, the lap shear strength taken after an incubation of 2 minutes in PBS and an incubation of 24 hours in PBS, in some embodiments. The p value was calculated with a student's t-test in Microsoft Excel (2 array, 2 tails, 2 type). * p≥0.05, ** p≤0.05, *** p≤0.01. An almost two-fold increase was observed between the 2 minutes of incubation and the 24 hours of incubation.

FIG. 3C illustrates a comparison of shear strength between a pristine solid adhesive patch and the patch also having an adhesive liquid spray, the comparison made on wet bovine pericardium taken after an incubation of 2 minutes in PBS and an incubation of 24 hours in PBS, in some embodiments. The p value was calculated with a student's t-test in Microsoft Excel (2 array, 2 tails, 2 type). * p>0.05, ** p≤0.05, *** p≤0.01. The addition of the spray should a very significant difference at 2 minutes, but there was no significant difference at 24 hours. Without intending to be bound by any theory or mechanism of action, we suspect that the high hydrophobicity and lower chain mobility in the solid state hindered the interaction of the polymer surface with wet tissue. Likewise, we suspect that the presence of highly dynamic covalent bonds in the network allows the reorganization of the network to obtain favorable interaction with the tissue surface; hence, the shear strength increases over time.

FIG. 3D illustrates a comparison of shear strength of the adhesive on wet bovine pericardium when applied using a spray application, brush application, and a solid adhesive patch, wherein, the shear strengths of all were compared to the shear strength of Dermabond, in some embodiments. The p value was calculated with a student's t-test in Microsoft Excel (2 array, 2 tails, 2 type). * p>0.05, ** p≤0.05, *** p≤0.01. The adhesive contains α-lipoic acid monomer (L1), an NHS-derivatized α-lipoic acid (L2) as a first stabilizer, and the conjugate base of the α-lipoic acid monomer (L4) to help stabilize the “precursor” from an undesired polymerization. At 2 minutes incubation in PBS the adhesion strengths between the 2 different adhesives are comparable. At 24 hours incubation in PBS, the spray adhesive was significantly stronger than the brush adhesive, assumed due to the lower carboxylic acid concentration in the stable liquid precursor used in the brush adhesive due to the addition of the conjugate base of the lipoic acid (L4). Even with the lower adhesion strength, however, the brush adhesive remains comparable to the medical grade cyanoacrylate adhesive.

FIG. 3E illustrates a comparison of shear strength of the adhesive when bonding between wet bovine pericardium and several different surfaces after 5 minutes incubation in PBS, the surfaces being stainless steel (SS), titanium dioxide (TiO2), silicate glass (SiO2), Nylon, high density polyethylene (HDPE), Polydimethylsiloxane (PDMS, a silicone polymer), and Poly(tetrafluoroethylene) (PTFE, TEFLON), in some embodiments. The p value was calculated with a student's t-test in Microsoft Excel (2 array, 2 tails, 2 type). * p>0.05, ** p≤0.05, *** p≤0.01. Interestingly, and surprisingly excellent adhesion strengths were observed with both metallic and polymeric substrates, showing a remarkable ability of our adhesive to mediate bonding of various medical devices to tissue. Moreover, the high adhesion strength of tissue with polymeric substrates like PE and PTFE opens the possibility of using the adhesives taught herein in vascular grafting.

FIG. 3F illustrates a comparison of burst strength of a solid adhesive patch and liquid spray combination on wet pig skin, stomach, and intestine after an incubation of 2 minutes in PBS and an incubation of 24 hours in PBS, in some embodiments. The p value was calculated with a student's t-test in Microsoft Excel (2 array, 2 tails, 2 type). * p>0.05, ** p≤0.05, *** p≤0.01. The data shows at least that the patch, with or without the addition of liquid adhesive before application, provides a rapid and effective tissue adhesion and sealing without burst.

FIG. 3G illustrates a comparison of shear strength comparison with and without non-adhesive spray containing a PEG-derivatized poly(α-lipoic acid) on a wet bovine pericardium after an incubation of 2 minutes in PBS and an incubation of 24 hours in PBS, in some embodiments. The p value was calculated with a student's t-test in Microsoft Excel (2 array, 2 tails, 2 type). * p>0.05, ** p≤0.05, *** p≤0.01. The data shows at least that use of a lipoic-acid-functionalized, antifouling polymer, on top of an in-situ formed poly(α-lipoic acid) adhesive could be used to prevent unwanted biofouling or post-surgical adhesion formation.

FIG. 3H illustrates the polymerization and depolymerization of poly(α-lipoic acid) using glutathione (GSH)-mediated degradation of the polymer, in some embodiments. The p value was calculated with a student's t-test in Microsoft Excel (2 array, 2 tails, 2 type). * p>0.05, ** p≤0.05, *** p≤0.01. The data shows at least that the polymers are recyclable, and can be recycled efficiently due to the ability to degrade efficiently from the polymer form to the individual monomers.

FIGS. 31 and 3J illustrate effective patching of a punctured porcine stomach with a solid adhesive patch, in some embodiments. FIG. 3I shows the puncture, and FIG. 3J shows the successful patching as tested for air leaks with a soap water spray, in some embodiments. The p value was calculated with a student's t-test in Microsoft Excel (2 array, 2 tails, 2 type). * p>0.05, ** p≤0.05, *** p≤0.01 . . .

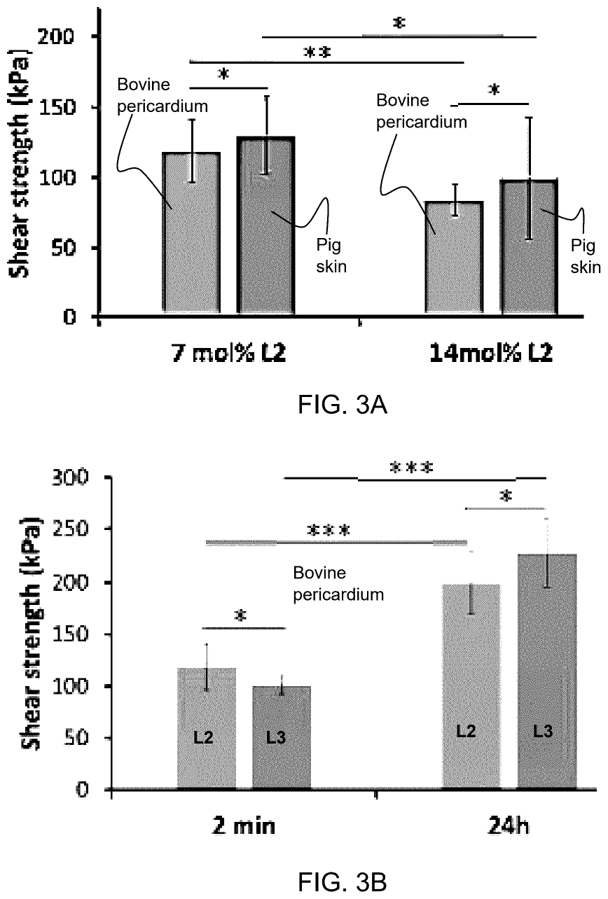

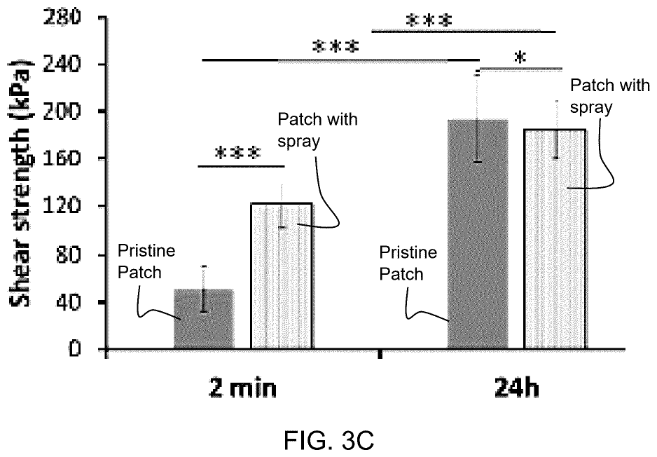

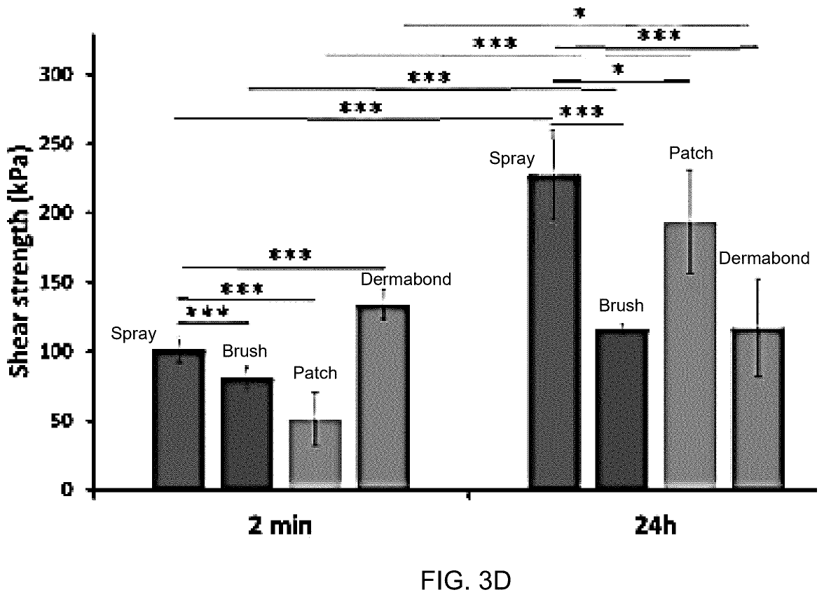

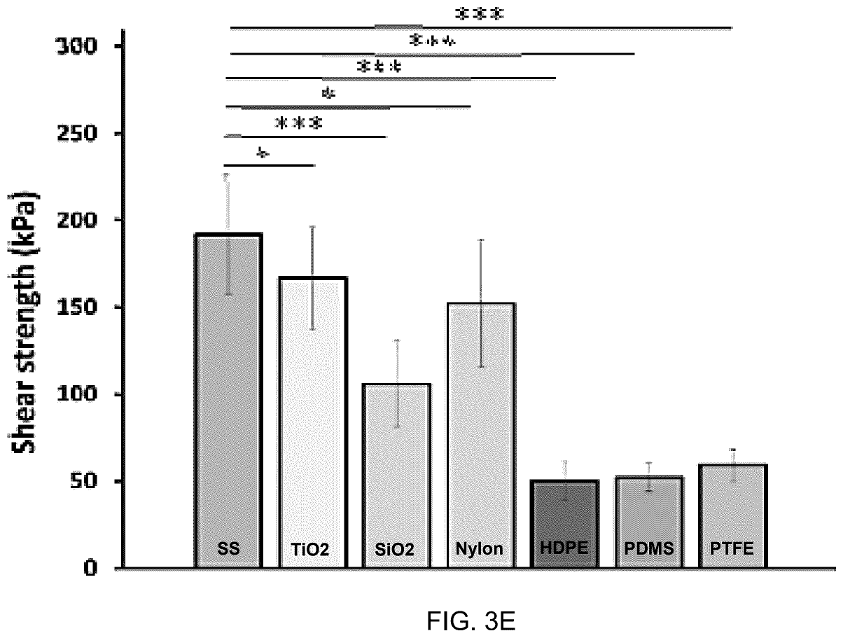

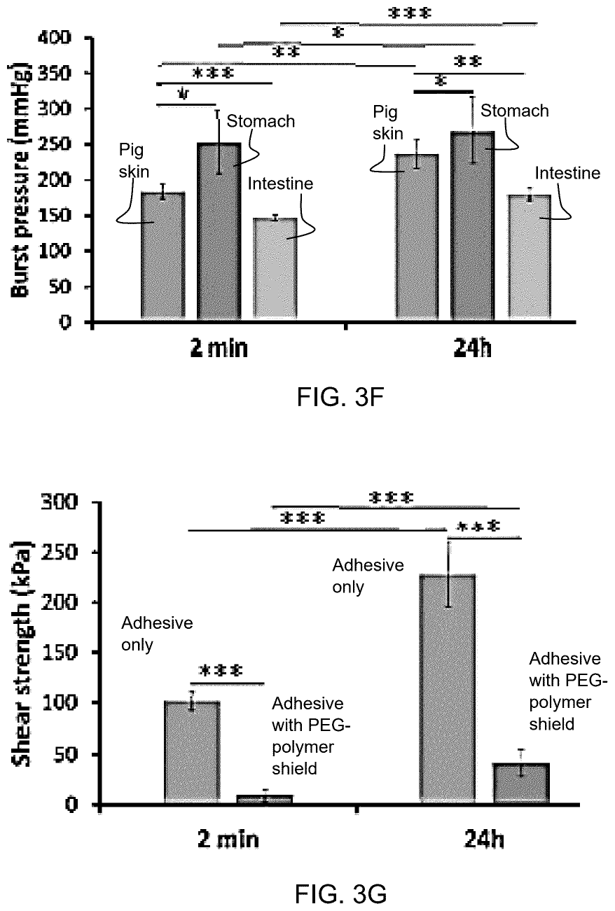

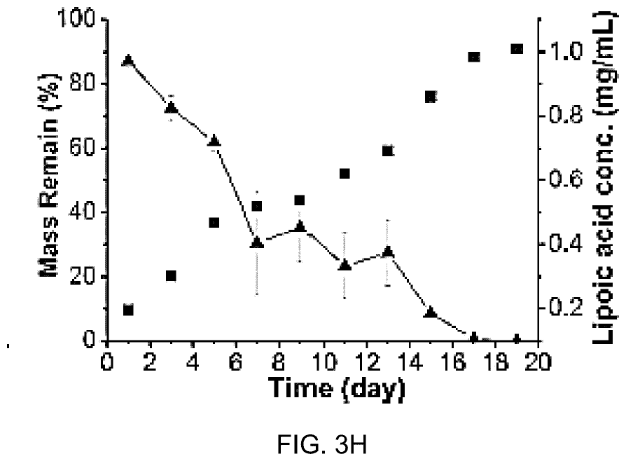

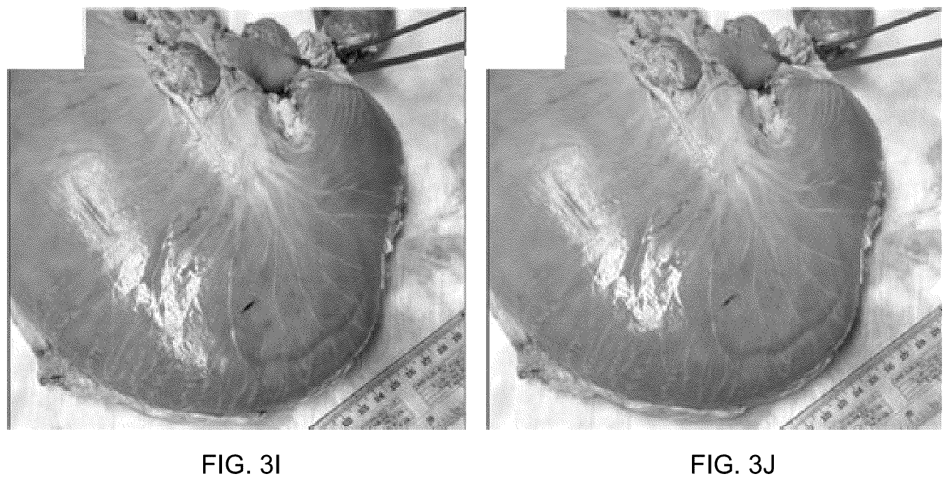

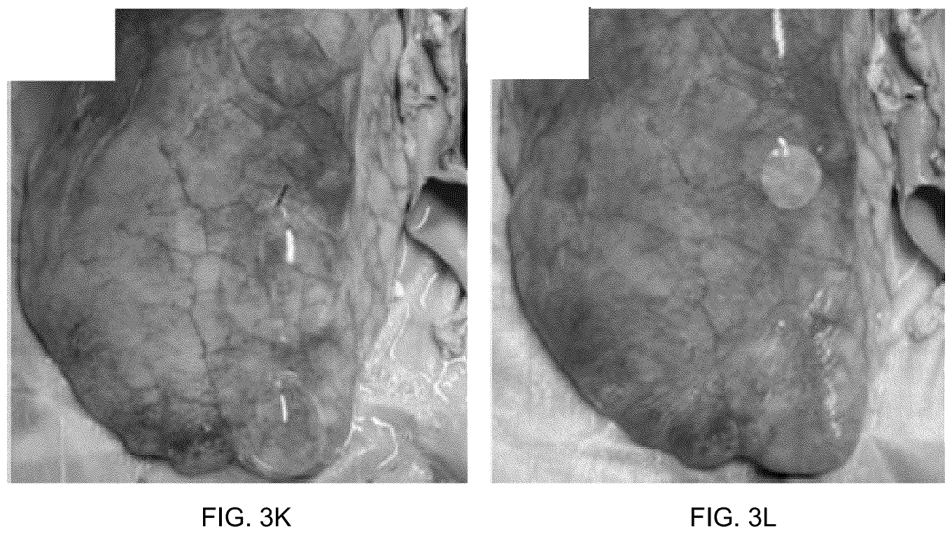

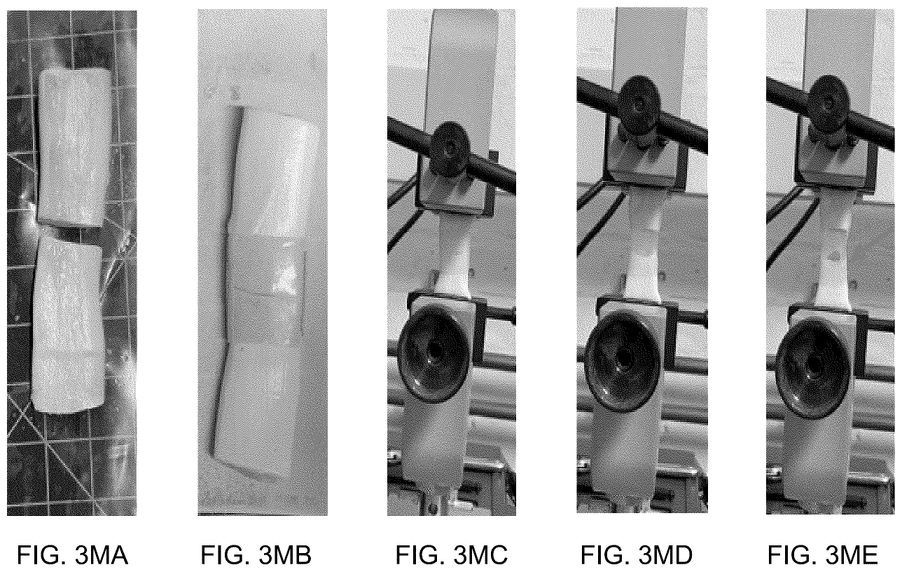

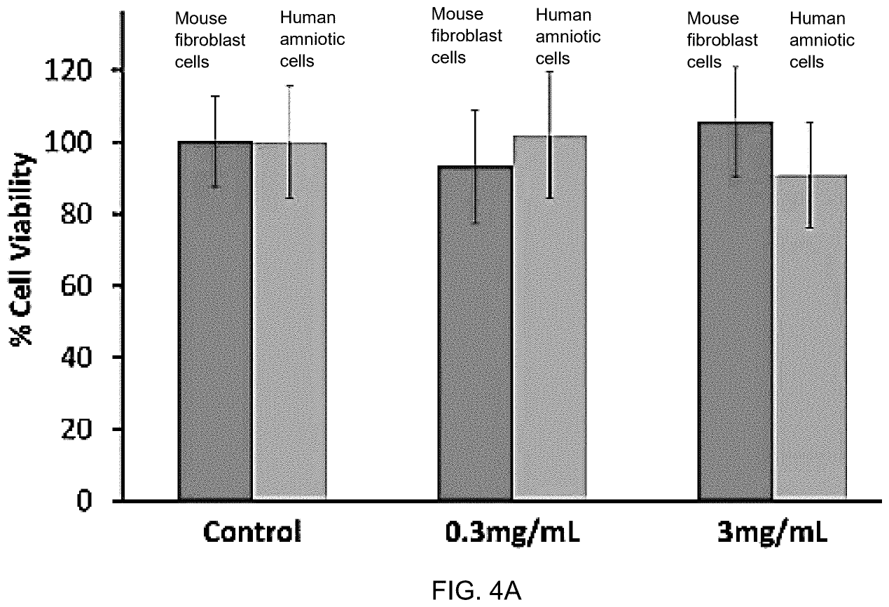

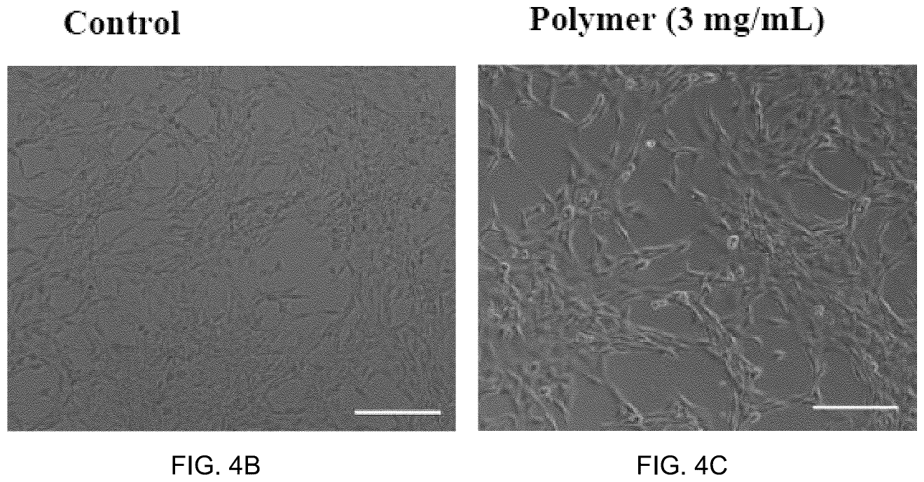

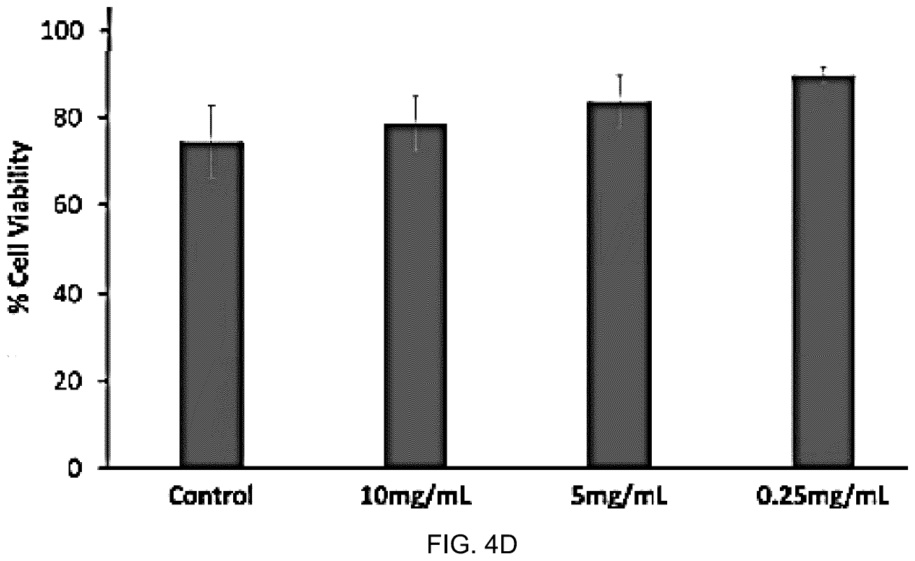

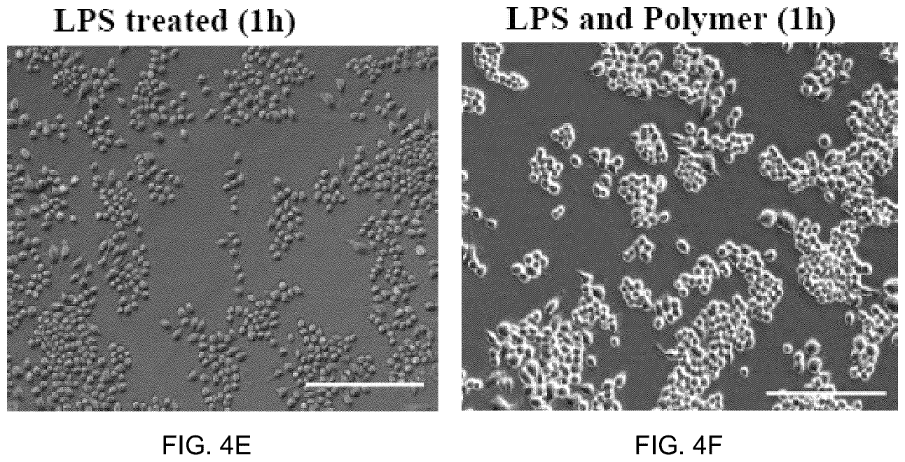

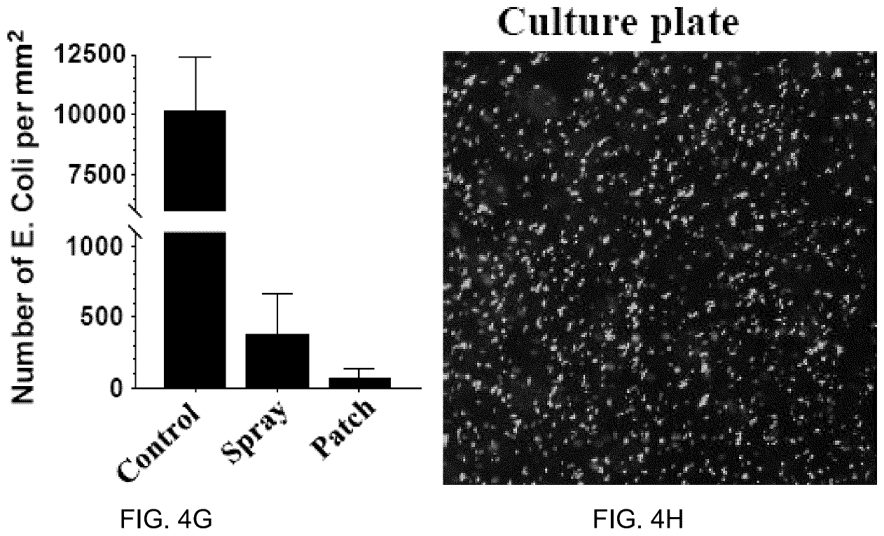

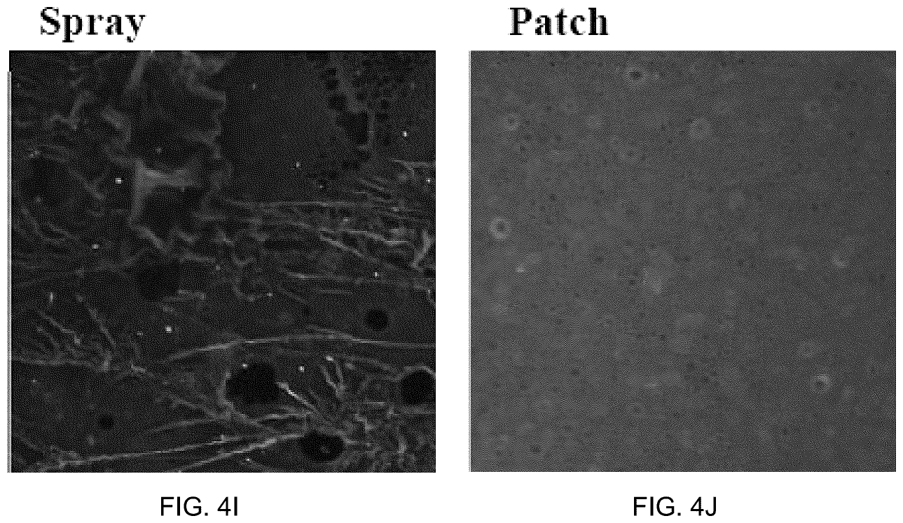

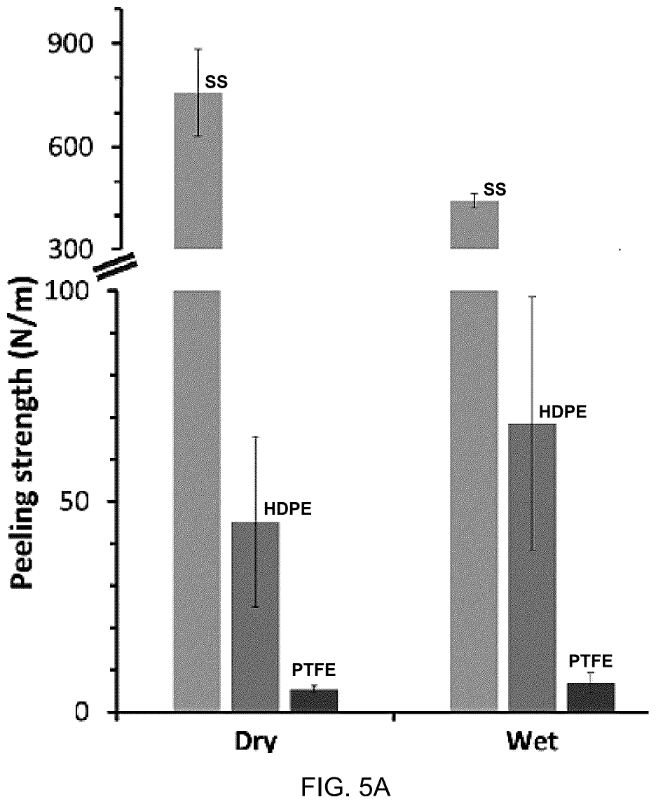

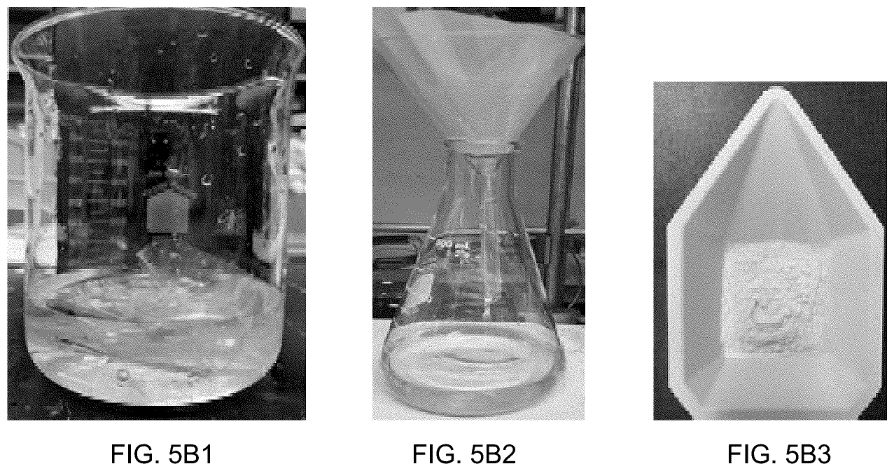

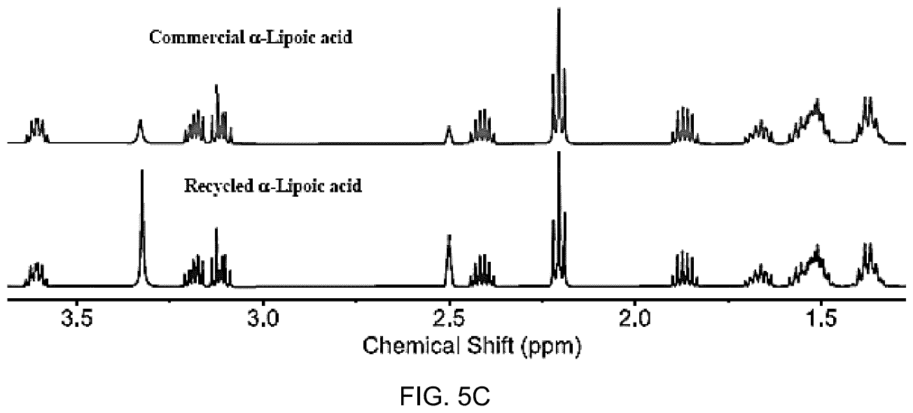

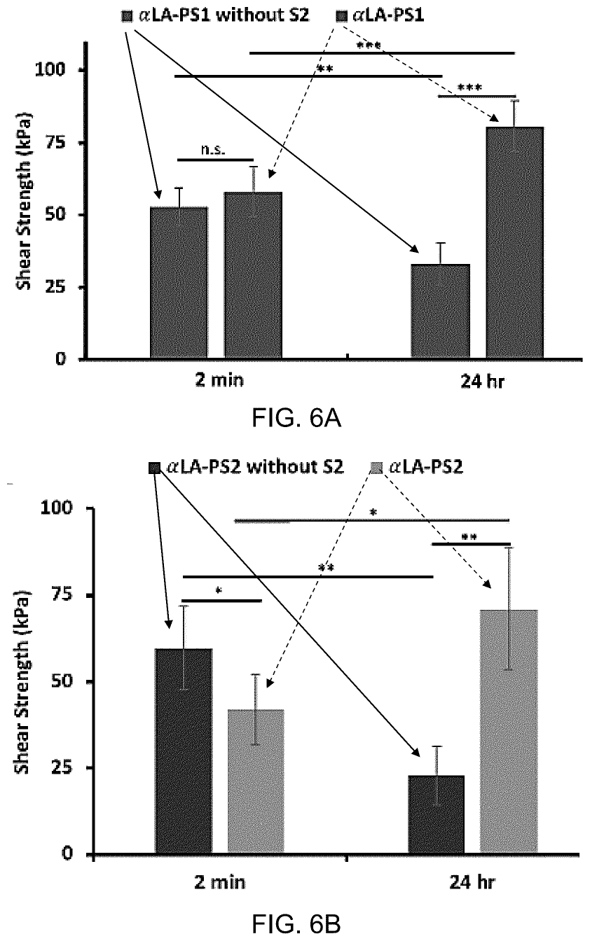

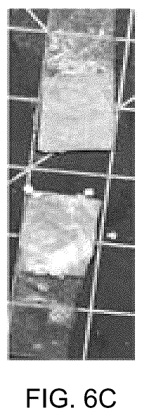

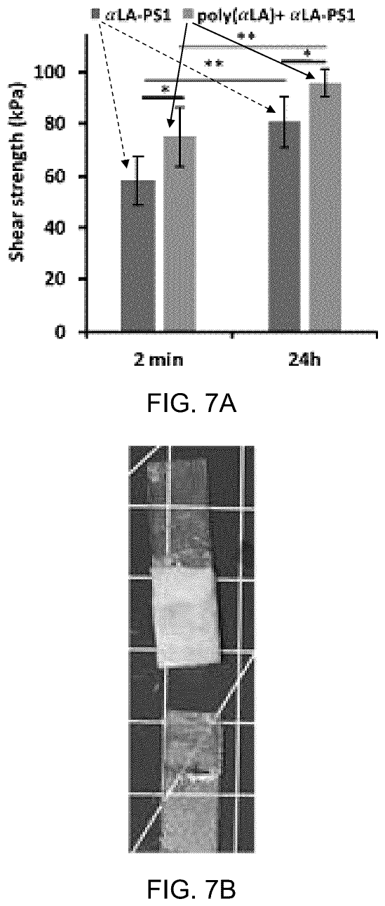

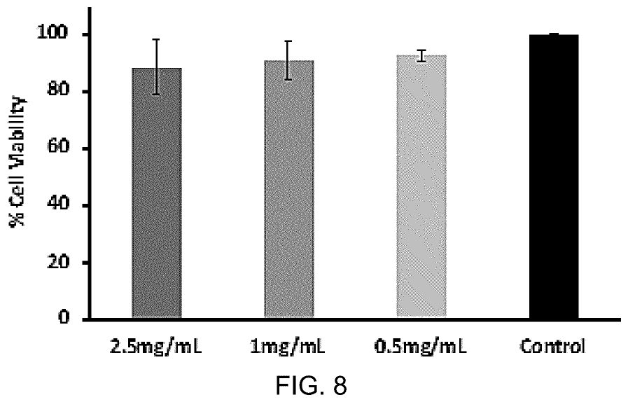

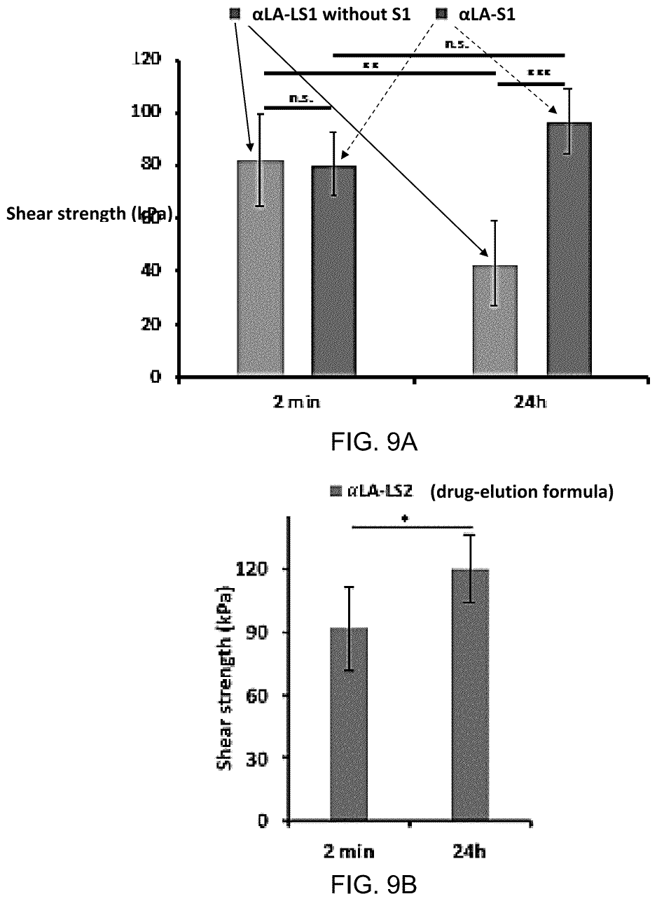

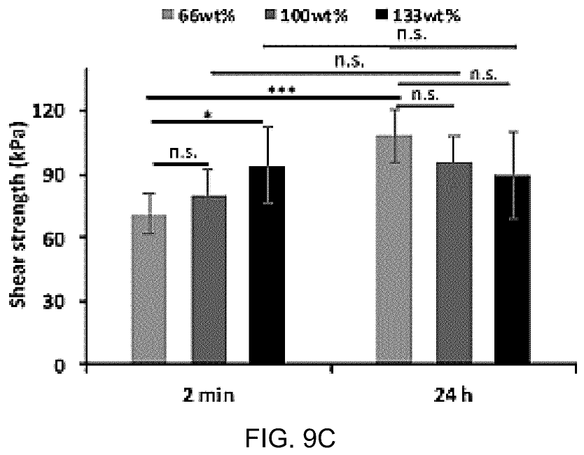

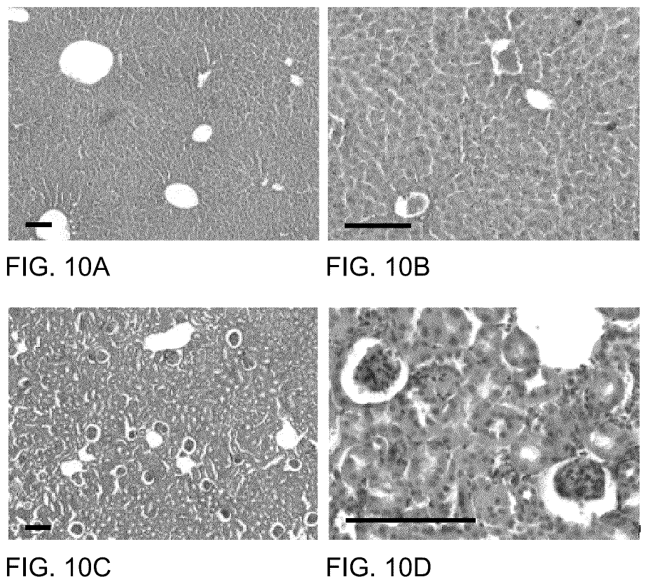

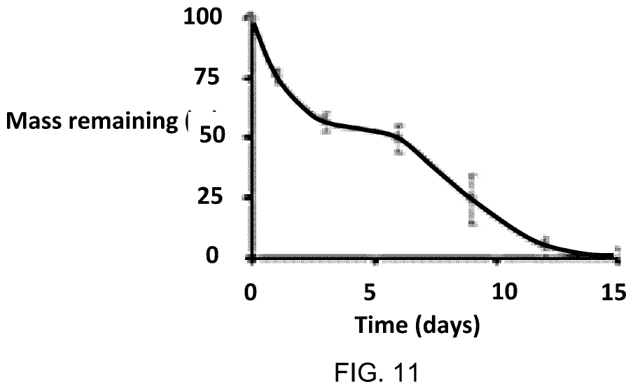

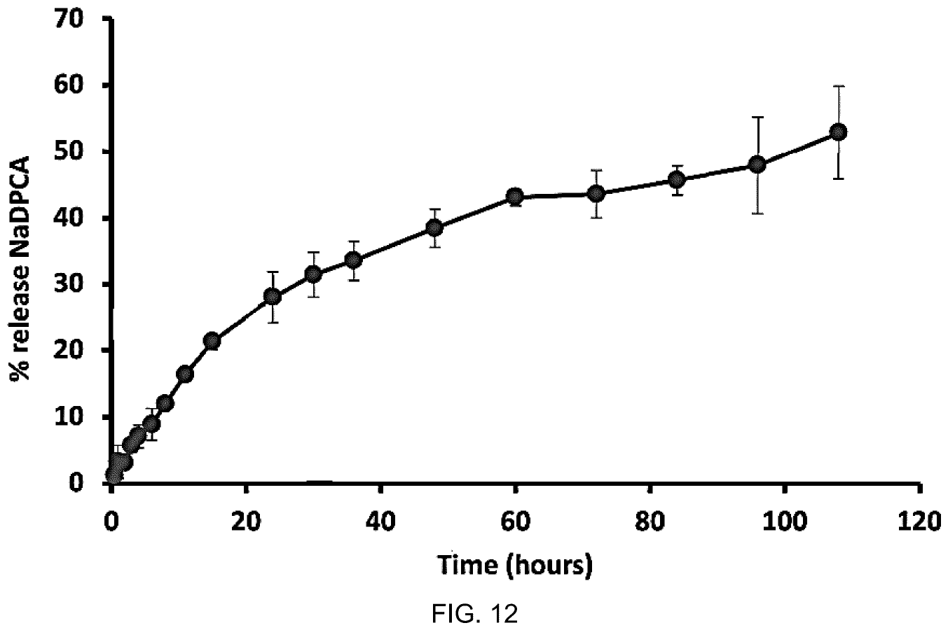

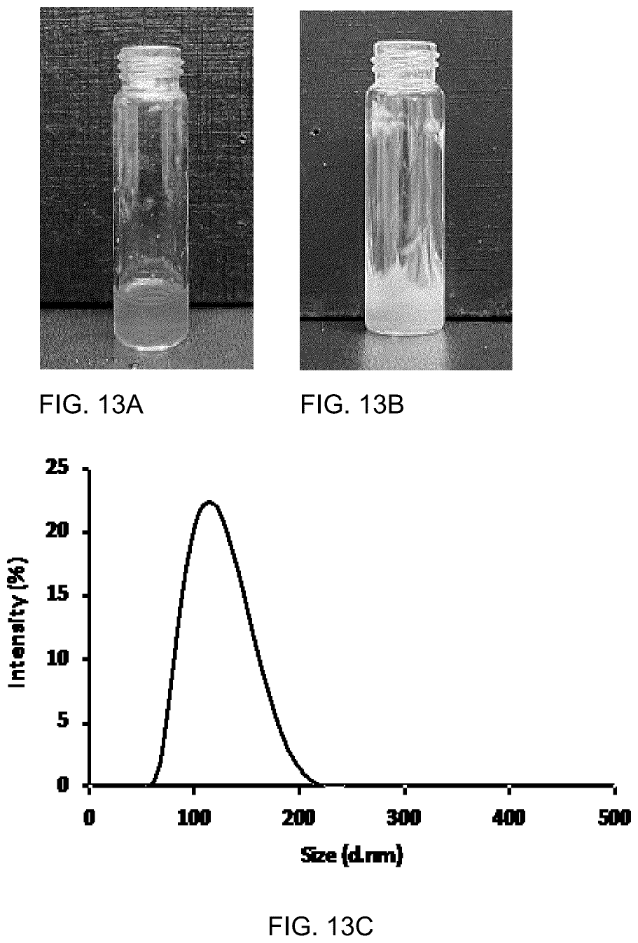

FIGS. 3K and 3L illustrate effective patching of a punctured porcine lung with a solid adhesive patch, in some embodiments. FIG. 3K shows the puncture, and FIG. 3L shows the successful patching as tested for air leaks with a soap water spray, in some embodiments. The p value was calculated with a student's t-test in Microsoft Excel (2 array, 2 tails, 2 type). * p>0.05, ** p≤0.05, *** p≤0.01. The patched lung puncture was tested for air leaks with a soap and water spray and none were found.

FIGS. 3MA-3ME illustrate adhesion of a patch to a bovine tendon, in some embodiments. FIG. 3MA shows a completely dissected bovine tendon. FIG. 3MB shows a wrapping of the transected tendon with a single layer of 2 cm wide patch sprayed ethanolic precursor. FIG. 3MC is a photograph of the wrapped transected tendon loaded in the instrument, before tensile extension at a crosshead speed of 50 mm/min after 5 min incubation in 1×PBS at 37° C. FIG. 3MD is a photograph taken in the middle of the tensile extension and shows elongation of the polymer patch without any interfacial failure. FIG. 3ME is a photograph taken at the end of the tensile extension and shows cohesive rupture (indicated by the arrow) of the patch between two tendon segments, while the patch remains attached to tendon segments. Cohesive rupture of the patch was observed under tensile extension, while the adhesive remains attached to the tendon.

FIG. 4A illustrates an in-vitro biocompatibility profile of in-situ polymerized spray adhesive with mouse fibroblast cells (NIH 3T3) and human amniotic cells after 24 hrs co-culture of the cells with the poly(α-lipoic acid) polymers, in some embodiments. A high density of cells, comparable to growth media controls was observed after the 24 h co-culture with the synthesized polymers, indicating a high biocompatibility of the polymers with the cells.

FIGS. 4B and 4C illustrates comparison images of an in-vitro biocompatibility profile of in-situ polymerized spray adhesive with mouse fibroblast cells (NIH 3T3) and human amniotic cells after 24 hrs co-culture of the cells with the 3 mg/ml concentration of poly(α-lipoic acid) polymers, in some embodiments. The images also show a high density of cells, comparable to growth media controls was observed after the 24 h co-culture with the synthesized polymers, indicating a high biocompatibility of the polymers with the cells. The scale bar is 200 μm.

FIG. 4D illustrates a relative cell viability in an in-vitro biocompatibility profile of a solid adhesive patch with mouse fibroblast cells (NIH 3T3) and human amniotic cells after 24 hrs co-culture of the cells with the solid adhesive patch, in some embodiments. A high density of cells, comparable to growth media controls was observed after the 24 h co-culture with the synthesized polymers, indicating a high biocompatibility of the polymers with the cells.

FIGS. 4E and 4F tests the antioxidant property of in-situ synthesized poly(α-lipoic acid) polymers in the presence of macrophages in an lipopolysaccharide (LPS)-induced oxidative stress model, in some embodiments. The macrophages were treated with LPS for 1 hour and imaged for the presence of reactive oxygen species (ROS) as shown FIG. 4E, and that image was compared to an image in FIG. 4F of the macrophages treated with LPS in the presence of the poly(α-lipoic acid) for 1 hour. Green dots are seen in FIG. 4E which is a formation of reactive oxygen species (ROS), whereas the formation of the ROS was completed suppressed in the presence of the poly(α-lipoic acid) as shown by the absence of the green dots in FIG. 4F. The scale bar is 200 μm.

FIGS. 4G-4J test the bacterial resistance of the adhesive compositions by illustrating a comparison of a control assay of E. coli growth in a culture plate to the E. coli growth on a solid adhesive patch and a culture plate coated with the in-situ synthesized poly(α-lipoic acid) polymer. As shown in FIG. 4G, the adhesive compositions serve very well as a bacterial barrier after 24 hours co-culture. FIG. 4H shows the presence of the cells in the control, whereas FIGS. 41 and 4J show how the presence of the cells is reduced on both the solid adhesive patch and the polymer coated culture plate due to the bacterial resistance properties of the adhesive composition.

FIGS. 5A, 5B1-B3, and 5C show the results of a test of the adhesive strength, recycling, and chemical profile of a pressure sensitive adhesive (PSA) produced from a poly(α-lipoic acid) polymer adhesive composition, in some embodiments. FIG. 5A compares peeling strength of the PSA against stainless steel (SS), high density polyethylene (HDPE), and polytetrafluoroethylene (PTFE) at dry and underwater conditions, showing the adhesion works almost as well underwater as when used in dry conditions, and that although the adhesion strength varies between substrates, the PSA did adhere to all of the substrates, where the adhesion strength order is generally SS>>HDPE>>PTFE.

FIG. 5B1-B3 show a recycling sequence of the PSA. FIG. 5C provides an ¹H NMR comparison between the chemical spectra of commercial vs. recycled α-lipoic acid, showing the recycling process is successful.

FIGS. 6A-6C illustrate how the adhesive strength of the powder adhesives relies on the addition of stabilizer with the passage of time from 2 minutes to 24 hours, according to some embodiments. The data suggests that regardless of any effect that the ratio of αLA:NaLA may have on adhesion at 2 minutes, there appears to be significantly more adhesion in the presence of stabilizer, regardless of whether the stabilizer is S1 or S2, and the failure appears to be a cohesive failure rather than an adhesive failure.

FIGS. 7A-7B illustrate how the adhesive strength of the powder adhesives affect the adhesive strength of a poly(αLA) patch on bovine pericardium tissue, according to some embodiments. Decorating the patch increases adhesive strength appreciably.

FIG. 8 illustrates that αLA-PS1 is biocompatible with NIH 3T3 (murine fibroblasts) cells at concentrations of 2.5 mg/ml, 1.0 mg/ml, and 0.5 mg/ml, according to some embodiments.

FIGS. 9A-9C illustrate how the adhesive strength of the powder adhesives relies on the addition of stabilizer with the passage of time from 2 minutes to 24 hours, how the addition of 2.34% NaDPCA has no effect on adhesion and, in fact, might surprisingly add adhesive strength, and the concentration of water has an appreciable effect that inverts from 2 minutes to 24 hours, according to some embodiments.

FIGS. 10A-10D illustrate H&E-stained images of liver tissue (FIGS. 10A and 10B) and kidney tissue (FIGS. 10C and 10D) from mice that received subcutaneous αLA-LS1 impants, according to some embodiments. All organ tissue appear healthy with no evidence of anomalies resulting from polymer implantation. The bar scale represents 100 μm. These figures illustrate that αLA-LS1 is highly biocompatible in mice, according to some embodiments.

FIG. 11 illustrates the biodegradation of the liquid adhesive, showing that a complete degradation occurred in about two weeks, in some embodiments.

FIG. 12 illustrates the drug elution curve of a liquid adhesive composition eluting NaDPCA over several days, according to some embodiments.

FIGS. 13A-13C show that the NaDPCA appears to be present as a nanostructure in view of the opacity it creates in a liquid precursor for αLA-LS1, and the dispersion plots obtained using dynamic light scattering analysis. FIG. 13A is a photograph of the liquid precursor αLA-LS1 without drug, FIG. 13B is the liquid precursor αLA-LS1 with drug showing opacity from drug particles, and FIG. 13C is a dispersion plot of a Dynamic light scattering analysis of the opaque NaDPCA, αLA and NaLA (0.1 mg/mL) in 1:1 ethanol-water mixture. Dispersion plots were obtained for NaDPCA/αLA particles at concentration of 0.1 mg/mL. Z_{avg} 137±8 nm; Pdi 0.240±0.017.

### DETAILED DESCRIPTION OF THE INVENTIONS

The teachings provided herein include adhesive compositions for medical uses. The compositions can be liquid, powder, solid, or a combination thereof. The adhesive compositions can include cyclic disulfide monomers, oligomers of reactions between the cyclic disulfides, polymers of the cyclic disulfides, and any combination thereof. Methods of making and using the adhesive compositions are included for medical applications, and can include uses on any of a variety of substrates that include biological tissue; biomaterials, including synthetics and natural such as bone, wood, and cellulosics; metals and alloys; polymers, plastics, and rubbers; ceramics; composites; and, any combination of these materials.

The adhesives can also adhere to substrates in any of a variety of environmental conditions that include ambient atmospheric conditions, wet conditions that include adhering materials underwater, and in a wide variety of temperatures and pressures, such as those temperatures and pressures found in medical environments, including in living tissue. Bioactive agents can be delivered to a target tissue by the adhesive compositions, allowing for the design of many different types of medicinal compositions, including medicinal adhesive compositions that can be applied to a variety of medical products and uses, both topical and internal, including surgical uses. It should be appreciated that the compositions provided herein will be useful in a plethora of medical applications for a subject or patient. The terms “subject” and “patient” can be used interchangeably and refer to an animal such as a mammal including, but not limited to, non-primates such as, for example, a cow, pig, horse, cat, dog, rat, and mouse; and primates such as, for example, a monkey, or a human. One of skill will appreciate that the terms “agent”, “active agent”, “bioactive agent”, “biologically active agent”, “biologic”, “pharmaceutical”, “pharmaceutically active agent”, and “drug” can be used interchangeably in embodiments taught herein.

A “bioactive agent” can be a moiety, for example, that can be combined with a polymer and provides a therapeutic effect, a prophylactic effect, both a therapeutic and a prophylactic effect, or other biologically active effect within a subject. Moreover, the bioactive agents of the present invention may remain linked to a portion of the polymer or be released from the polymer. A “diagnostic agent” is a type of bioactive agent that can be used, for example, in diagnosing the presence, nature, or extent of a disease or medical condition in a subject. In one embodiment, a diagnostic agent can be any agent that may be used in connection with methods for imaging an internal region of a patient and/or diagnosing the presence or absence of a disease in a patient. Diagnostic agents include, for example, contrast agents for use in connection with ultrasound imaging, magnetic resonance imaging (MRI), nuclear magnetic resonance (NMR), computed tomography (CT), electron spin resonance (ESR), nuclear medical imaging, optical imaging, elastography, and radiofrequency (RF) and microwave lasers. Diagnostic agents may also include any other agents useful in facilitating diagnosis of a disease or other condition in a patient, whether or not imaging methodology is employed.

Many of the adhesive compositions taught herein have been tested using α-lipoic acid monomers which are a preferred monomer for many uses due to the properties of the monomers, as well as the properties of the poly(α-lipoic acid) polymers. Asparagusic acid monomers have many desired properties, as do the poly(asparagusic acid) polymers. Like α-lipoic acid, we developed a method of polymerizing stable forms of poly(asparagusic acid) and found that it had excellent mechanical properties and adhesion strength, some of which are superior to poly(α-lipoic acid) as well as a cost-effective production and closed-loop chemical recycling. An added benefit is that poly(asparagusic acid) (L8) is that, although it also depolymerizes on it's own, it depolymerizes more slowly than poly(α-lipoic acid) because it's cyclic ring has more strain and, although it can certainly be stabilized like the poly(α-lipoic acid), the stability of poly(asparagusic acid) without a stabilizer molecular is sufficient for many uses. Like poly(α-lipoic acid), poly(asparagusic acid) also readily depolymerizes into safe monomers and oligomers, the monomers having the therapeutic activities of interest.

Generally speaking, the adhesive compositions include cyclic disulfide monomer components that polymerize into a sticky polydisulfide polymers at the time of use; oligomers of the cyclic disulfide that polymerize into a sticky polydisulfide polymers at the time of use; polydisulfide polymers of the cyclic disulfide components ready for such use; solid adhesive products formed from such sticky polymers; any one or combination of those for additional polymerization before or during such uses; and, any combination thereof.

The polymerization of the cyclic disulfide monomer can occur in water; an aqueous solution; a water miscible solvent; a polar, protic solvent; thermally; and, any combination thereof. For purposes of efficiency, all solvents will be collective referred to as “reaction solvent”. Ethanol, water, a combination of ethanol and water, any aqueous media such as saline or a buffer solution, and the like, are examples of polar, protic solvents. DMSO and DMF are examples of solvents miscible in water, and both are polar, aprotic solvents. Thermal polymerization can occur in a dried mixture of monomers, or a mixture of monomers in a reaction solvent. In some embodiments, the reaction solvents can be selected from the group of ethanol, DMSO, methanol, polyethylene glycol, glycerol, ionic liquids, vegetable oils, or any water-miscible organic solvents.

The polymerization of the cyclic disulfide monomer can occur in a reaction solvent to create a sticky polymer. In some embodiments, an electrophilic stabilizer can be added for stabilizing such sticky polymers and address the problem of depolymerization that happens in polymers created by the cyclic disulfide monomers. The depolymerization can render some polymers useless in applications that require at least a minimal amount of polymer stability that cannot be obtained without the addition of the stabilizer. In some embodiments, a terminator molecule is added for further stabilizing the polymer. In some embodiments, a Lewis acid or a Bronsted acid is added to increase the rate of polymerization. In some embodiments, dimethyl sulfoxide (DMSO) is added to increase the rate of the polymerization. In some embodiments, a Lewis acid or a Bronsted acid is addition with a catalytic amount of the DMSO to increase the rate of polymerization. In some embodiments, the conjugate base of a cyclic disulfide monomer is added to increase the stability of the adhesive compositions as a whole before use of the compositions as intended. And, in some embodiments, a crosslinker is added to strengthen polymeric adhesives formed from the adhesive compositions through covalent bond or non-covalent bonds made between the crosslinker and the adhesive polymers. The crosslinker can include, for example, a monomer, an oligomer, a polymer, and conjugate bases of monomers used in the polymerization; metal ions, such as multivalent metal ions including divalent metal ions such as Ca++; transition and alkali earth metal salts; and, metal salts of cyclic 1,2-dithiolane derivatives; and, combinations thereof; wherein, the crosslinkers are added to further strengthen the compositions.

### The Compositions

### The Cyclic Disulfide Monomers

The cyclic disulfide monomers polymerize to relieve the ring strain. In some embodiments, the cyclic disulfide monomers are a plurality of polymerizable monomers selected from the group consisting of substituted cyclic disulfide molecules having from 2 to 4 C atoms in a cyclic ring with a disulfide bond, the substituted cyclic disulfide molecules functional to polymerize through a ring-opening reaction in a polar, protic solvent to form a polymer having
a first active thiol end and a second active thiol end; and,
a plurality of repeating units having a substituted dithioalkyl structure from the ring opening reaction as follows

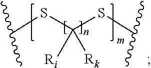

wherein,
n is an integer ranging from 2 to 4;
each Rᵢ and Rₖ is independently selected from the group consisting of H; alkyl, cycloalkyl, alkenyl, alkynyl, and aryl groups having from 1-8 carbons, 1-6 carbons, 1-4 carbons, 1-3 carbons, 2 carbons, or 1 carbon; hydroxylated alkyl, cycloalkyl, alkenyl, alkynyl, and aryl groups having from 1-8 carbons, 1-6 carbons, 1-4 carbons, 1-3 carbons, 2 carbons, or 1 carbon; and, carboxylated alkyl, cycloalkyl, alkenyl, alkynyl, and aryl groups having from 1-8 carbons, 1-6 carbons, 1-4 carbons, 1-3 carbons, 2 carbons, or 1 carbon; wherein, i and k are integers and at least one Rᵢ or Rₖ in each repeating unit includes a carbonyl functionality;
and,
m is an integer selected to match the desired molecular weight of the polymer.

The molecular weight of the polymer can be any desired molecular weight, selected by the skilled artisan to obtain desired chemical and physical behavior of the polymer for it's intended uses. The molecular weight can be selected reflect the desired length of the polymer, so the molecular weight for a given length can vary depending on the size of the desired substituents of the polymer, and the degree of branching of the polymer, for example. In some embodiments, the molecular weight of the polymer can range from 1000 Daltons to 100,000 Dalton, from 5000 Daltons to 500,000 Daltons, from 6000 Daltons to 400,000 Daltons, from 7000 Daltons to 300,000 Daltons, from 8000 Daltons to 200,000 Daltons, from 9000 Daltons to 150,000 Daltons, from 10,000 Daltons to 100,000 Daltons, from 20,000 Daltons to 100,000 Daltons, from 30,000 Daltons to 100,000 Daltons, from 40,000 Daltons to 100,000 Daltons, from 10,000 Daltons to 40,000 Daltons, from 15,000 Daltons to 40,000 Daltons, from 20,000 Daltons to 40,000 Daltons, or any molecular weight or range of molecular weights therein in increments of 500 Daltons.

In some embodiments, the cyclic disulfide monomer can be, for example, a dithietane (2 carbons in the ring), a dithiolane (3 carbons in the ring), a dithiane (4 carbons in the ring), and any substituted form thereof or combination thereof. In some embodiments, the cyclic disulfide can be 1,2-dithietane, and substituted forms thereof; 1,2-dithiolane, and substituted forms thereof; 1,2-dithiane, and substituted forms thereof; and any combinations thereof.

The cyclic disulfide monomer can substituted. In some embodiments, the plurality of substituted cyclic disulfide molecules include a substituted 1,2-dithietane, and the repeating unit includes a substituted 1,2-dithioethyl structure. In some embodiments, the plurality of substituted cyclic disulfide molecules is substituted 1,2-dithiolane, and the repeating unit is a substituted 1,3-dithioethyl structure. In some embodiments, the plurality of substituted cyclic disulfide molecules is a substituted 1,2-dithiane, and the repeating unit is a substituted 1,4-dithioethyl structure.

In some embodiments, the cyclic disulfide monomer can be a dithiolane monomer having the following general structure:

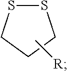

and, the R-group can be selected from the group consisting of the following, in some embodiments:

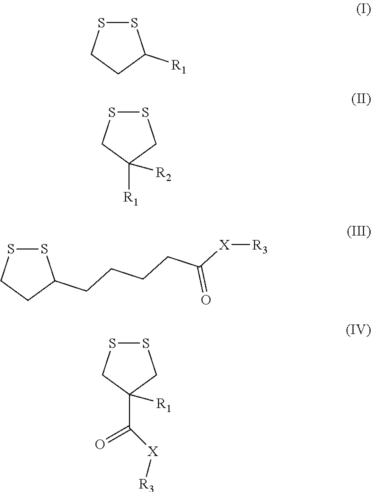

wherein,
R₁ can be selected from the group consisting of H; a linear, branched or cyclic saturated or unsaturated C1-C8 alkyl group, C1-C6 alkyl group, C1-C4 alkyl group, C1-C3 alkyl group, C2 alkyl group, or C1 alkyl group; a linear, branched or cyclic saturated or unsaturated C1-C8 alkyl group with a terminal carbonyl group, including smaller versions thereof that include, C1-C6 alkyl group, C1-C4 alkyl group, C1-C3 alkyl group, C2 alkyl group, or C1 alkyl group; a linear, branched or cyclic saturated or unsaturated C1-C8 with terminal mono or dicarboxylic acid, including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated C1-C8 with terminal mono or dicarboxylic salt, where the counter ion of carboxylate includes, but is not limited to, alkali metal ions (Na, K, Li), alkali earth metal ions (Ca, Mg), transition metal ions (Zn, Fe, V), including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated C1-C8 with terminal mono or dicarboxylic salt, where the counter ion of carboxylate includes, but is not limited to, an organic salt such as ammonium, phosphonium, imidazolium, choline, etc., including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated C1-C8 with mono or diester group, including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated C1-C8 with mono or diester group with an aromatic substituent; a linear, branched or cyclic saturated or unsaturated C1-C8 containing a mono or diamide group, including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated C1-C10 containing a mono or diamide and terminal carboxylic acid, including smaller versions thereof that include a C1-C8 alkyl group, a C1-C6 alkyl group, a C1-C4 alkyl group, a C1-C3 alkyl group, a C2 alkyl group, or a C1 alkyl group; a linear, branched or cyclic saturated or unsaturated C1-C8 containing a mono or thioester group; —(CH₂)₄CO₂H; —CO₂H; a linear, branched or cyclic saturated or unsaturated C1-C10 containing one or more cyclic 1,2-dithiolane derivatives; an organic small or macromolecule containing one or more cyclic 1,2-dithiolane derivatives; and combinations thereof;
R₂ can be selected from the group consisting of H; a linear, branched or cyclic saturated or unsaturated C1-C8 group, including smaller versions thereof that include a C1-C6 alkyl group, a C1-C4 alkyl group, a C1-C3 alkyl group, a C2 alkyl group, or a C1 alkyl group; and combinations thereof;
R₃ can be selected from the group consisting of H; a linear, branched or cyclic saturated or unsaturated C1-C8 group, including smaller versions thereof that include a C1-C6 alkyl group, a C1-C4 alkyl group, a C1-C3 alkyl group, a C2 alkyl group, or a C1 alkyl group; an aromatic or heterocyclic aromatic group; an alpha or beta amino acid; an alpha or beta peptide; dopamine; a monosaccharide, disaccharide or oligosaccharide; 1,4-dihydrophenonthrolin-4-one-3-carboxylic acid; a therapeutic molecule having a molecular weight of no more than 40,000 Daltons, 30,000 Daltons, 20,000 Daltons, 10,000 Daltons, 5000 Daltons, 3000 Daltons, 2000 Daltons, 1000 Daltons, 500 Daltons, or any range therein in increments of 100 Daltons. Any therapeutic molecule can be used, and in some embodiments, the therapeutic molecule can be selected from the group consisting of an antioxidant, an anti-inflammatory, an analgesic, an antiproliferant, an immunomodulator, an antimicrobial, an antibacterial, an antifungal, an antiviral, or any combination thereof; and,
X can be any moiety containing O, N, S, or any combination thereof. In some embodiments, X is a moiety selected from the group consisting of O, N, and S.

The cyclic disulfide monomer can substituted. In some embodiments, the plurality of substituted cyclic disulfide molecules include a substituted 1,2-dithietane, and the repeating unit includes a substituted 1,2-dithioethyl structure. In some embodiments, the plurality of substituted cyclic disulfide molecules is substituted 1,2-dithiolane, and the repeating unit is a substituted 1,3-dithioethyl structure. In some embodiments, the plurality of substituted cyclic disulfide molecules is a substituted 1,2-dithiane, and the repeating unit is a substituted 1,4-dithioethyl structure.

The cyclic disulfide monomer in an adhesive composition is the reactant that forms the repeating units in the polymer, and the polymer can be defined, at least in part, by a description of the repeating units. In some embodiments, the repeating unit is a substituted 1,3-dithiopropyl structure as follows:

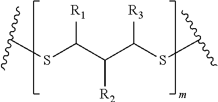

wherein,
m is an integer selected to match the desired molecular weight of the polymer;
Rᵢ includes R₁, R₂, and R₃; and,
each Rₖ is H;
and, each stabilizer molecule includes the substituted 1,3-dithiopropyl structure.

In some embodiments, the repeating unit has a substituted 1,3-dithiopropyl structure as follows

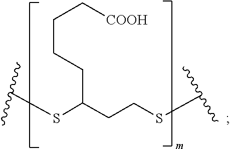

wherein,
m is an integer selected to match the desired molecular weight of the polymer;

R₁ is a pentanoic acid group, and R₂ and R₃ are each H;
and, each stabilizer molecule includes the substituted 1,3-dithiopropyl structure, wherein R₁ is a pentanoic acid group, and R₂ and R₃ are each H.

In some embodiments, the polymer further includes a plurality of terminator molecules selected from the group consisting of ethanol, a substituted 1,2-dithietane with a hydroxyl functionality, a substituted 1,2-dithiolane with a hydroxyl functionality, and a substituted 1,2-dithiane with a hydroxyl functionality, the terminator molecules functioning to form a second labile bond with the second active thiol end of the polymer to further stabilize the polymer.

In some embodiments, the repeating unit has a substituted 1,3-dithiopropyl structure

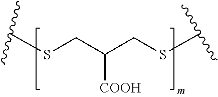

wherein,
m is an integer selected to match the desired molecular weight of the polymer;
R₁ and R₃ are H, and R₂ is a carboxylic acid group;
and, each stabilizer molecule includes the substituted 1,3-dithiopropyl structure, wherein R₁ and R₃ are H and R₂ is a carboxylic acid group.

### Reaction Solvents

The monomers polymerize to form an adhesive polymer upon contact with a reaction solvent, such as water, an aqueous solution, a polar protic solvent, or any water miscible solvent such as DMSO and DMF, which are polar aprotic solvents (all referred to herein as “reaction solvent”). The reaction solvent can be, for example, an alcohol, water, a mixture of an alcohol and water, pure ethanol, pure water, a mixture of ethanol and water, a buffer solution, dimethylsulfoxide (DMSO), dimethylformamide (DMF) polyethylene glycol (PEG), glycerol, ionic liquids such as saline, a vegetable oil, and the like, and combinations thereof.

In some embodiments, the reaction solvent can also be a biological fluid, or a biologic compatible fluid, for ease of direct use in medical applications, for example, blood, urine, sweat, amniotic fluid, spinal fluid, serum, plasma, isotonic saline, and cell culture media.

In some embodiments, buffers can also be used, either alone or in combination with any of the reaction solvents, the buffers including, for example, Phosphate-buffered saline (PBS), Dulbecco's Phosphate-Buffered Saline (DPBS), N-(2-Hydroxyethyl) piperazine-N′-(2-ethanesulfonic acid) (HEPES), 2-(N-morpholino) ethanesulfonic acid (MES), 2-(bis(2-hydroxyethyl)amino) ethane sulfonic acid (BES), 3-(N-morpholino) propanesulfonic acid (MOPS), 3-Morpholino-2-hydroxypropanesulfonic acid (MOPSO), Tricine, Bicine, [tris(hydroxymethyl)methylamino]propanesulfonic acid (TAPS), and the like, and combinations thereof.

### Stabilizers

There are at least two active thiol ends in the adhesive polymers taught herein, a first active thiol end, and a second active thiol end, where “first” and “second” only refer to two different positions, and do not suggest an order of reaction or relative reactivity. The stabilizers are electrophiles that bond to the first active thiol groups at an end of the polydisulfide polymers. It should be appreciated that, in some embodiments, the terms “stabilizer”, “stabilizing molecule”, “stabilizer group”, “stabilizing molecule”, and “stabilizing group”, and the like, can be used interchangeably, and refers to a chemical moiety that can bond to the first active thiol end. In some embodiments, the stabilizer can be one or more active agents. In some embodiments, the active agent can be selected from the group consisting of any active agent taught herein. In some embodiments, the one or more active agents can include one or more active agents desired to achieve an effect, whether the effect is a chemical effect, perhaps solubility or stability, a prophylactic effect, or a therapeutic effect.

In some embodiments, a plurality of stabilizer molecules can be configured to stabilize the first active thiol end of the polymer. The plurality of stabilizer molecules can include the substituted cyclic disulfide molecules derivatized to have a functional group that forms a first labile bond with the first active thiol end, the first labile bond selected from the group consisting of a thioester bond, a thiocarbamate bond, and a thioether bond. The bond can be selected to achieve a desired rate of biodegradation, a desired release rate of an active agent, a targeted drug delivery, or a combination thereof.

It should be appreciated that term “labile” is a term in the art of chemistry. In some embodiments, “labile” can refer to a bond that is cleavable by a biological mechanism inside the human body, such that a labile bond is one that adds to the biodegradable characteristic of an adhesive composition, or polymer, taught herein. In some embodiments, “labile” can refer to a bond that is cleavable by any chemical means that one of skill may use in a recycling process, such that the cleavage of the labile bond results in monomers that are the same or similar to, or substantially similar to, the original monomers reacted to form an original or regenerated adhesive composition or polymer taught herein, where “regenerated” can be used to refer to a polymer or material made from recycled monomers, and the term “substantially” and “substantially similar” can refer to a term of degree, where something that is “substantially the same” would be the same for all practical purposes to the ordinary skilled artisan upon a first consideration without further any further consideration needed. A molecule or material that appears “similar” to an ordinary skilled artisan may be considered to possibly have the desired function or activity but may need a second consideration or testing. A “substantially similar” molecule, on the other hand, would obviously have the desired function or activity in the view of the skilled artisan upon the first consideration. In some embodiments, the term “labile” can refer to a degradation of the adhesive composition or monomer from ambient environment conditions present in nature including the presence of water, heat, UV energy, bacteria, fungi, molds, enzymes, acids, bases, and the like, and any combination thereof. In some embodiments, the labile nature of bonds is a desired feature of biodegradation, a desired release rate of an active agent, a targeted drug delivery, or a combination thereof. In some embodiments, the labile nature of bonds is a desired feature of environmental hygiene and/or safety. In some embodiments, the labile nature of bonds is a desired feature for bio-elimination from a subject through ordinary filtration and excretion by the body. In some embodiments, the labile nature of bonds is a desired feature for ease of recycling by enabling the liberation of monomers from the recycled polymer.

The monomers used in the polymerization form the repeating units of the polymer. In some embodiments, the monomers and the stabilizer molecules are the same or substantially the same chemical moieties following a depolymerization of the polymer. This can be beneficial in a recycling process of the polymeric material because the monomer and the stabilizer are the same or similar molecules following depolymerization of the polymer. It should be appreciated that to be “the same” can be construed to mean identical, and so the terms “substantially” or “similar” are used to reflect the understanding that the skilled artisan would see a significant difference between the chemical moieties in a mixture. To substantially the same, or similar, for example, the chemical moieties (i) may only differ in structure in a way that is easily converted to be identical to the original monomer used in the reaction; (ii) may only differ in structure in a way that does not appreciably affect the repolymerization of the monomers to a regenerated material to have a desired function; (iii) may only differ in structure in a way that does not appreciably affect the repolymerization of the monomers to a regenerated material to have a desired biocompatibility; (iv) may only differ in structure in a way that does not appreciably affect the repolymerization of the monomers to a regenerated material to have a desired toxicity level; and/or (v) may have a desired yield in a repolymerization, regardless of whether a desired function is obtained in every repolymerization.

In some embodiments, the stabilizers include R—OH, R—NH, R—SH, R-carbonyls, R-thiocarbonyls, R-carboxyls, R-thiocarboxyls, R-esters, R-anhydrides, R-quinones, R-thioquinones, R-isocyanates, R-diisocyanates, R-imidazoles, and the like, where R can be any alkyl, cycloalkyl, alkenyl, alkynyl, and aryl groups having from 1-14 carbons; hydroxylated alkyl, cycloalkyl, alkenyl, alkynyl, and aryl groups having from 1-14 carbons; and, carboxylated alkyl, cycloalkyl, alkenyl, alkynyl, and aryl groups having from 1-14 carbons. In these embodiments, the stabilizers can include carbonyl imidazoles, N-hydroxy succinimide (NHS) esters, N-hydroxysulfosuccinimide esters, N-hydroxy phthalimide esters, N-hydroxy succinimide (NHS) esters of lipoic acid, and N-hydroxy succinimide (NHS) esters of asparagusic acid. The stabilizers can be configured for shape, size, and chemical activity, to control reactivity and function as desired, for example, to alter the strength of the labile bond with the active thiol end group, the strength of the stabilizer as a nucleophile, the strength of the stabilizer as an electrophile, steric hindrance, rate of reactivity, rate of polymerization, and the like. It should be appreciated that, in some embodiments, the number of carbons in any of the stabilizers can be configured by the skilled artisan to range from 1-14, 1-12, 1-10, 1-8, 1-7, 1-6, 1-5, 1-4, 1-3, 1-2, 1, or any amount or range of carbons therein.

In some embodiments, the stabilizers can be selected from the group consisting of

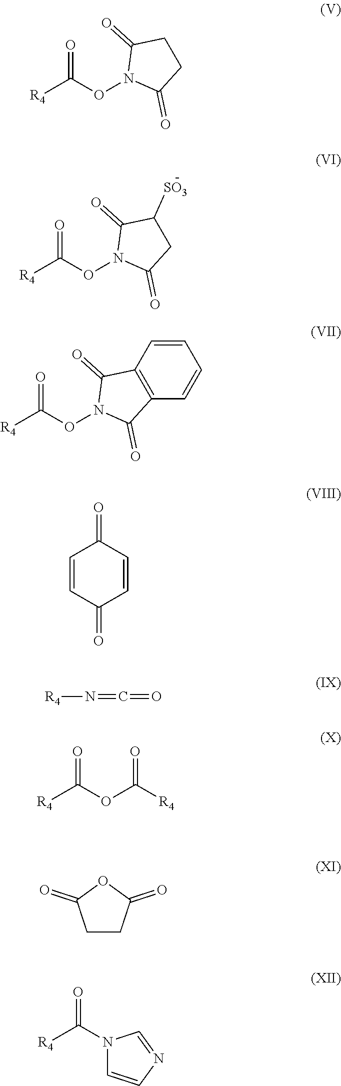

Namely, N-hydroxy succinimide (NHS) esters (V), N-hydroxysulfosuccinimide esters (VI), N-hydroxy phthalimide esters (VII), oxidized polyphenols, quinones (VIII), isocyanates (IX), linear or cyclic or poly anhydrides (X, XI), and carbonyl imidazoles (XII);

wherein, R₄ can be selected from the group consisting of an alkyl dithiolane; a poly dithiolane, OH; a linear, branched or cyclic saturated or unsaturated C1-C8 group, including smaller versions thereof that include a C1-C6 alkyl group, a C1-C4 alkyl group, a C1-C3 alkyl group, a C2 alkyl group, or a C1 alkyl group; a linear, branched or cyclic saturated or unsaturated macromolecules; an aromatic or heterocyclic aromatic group; and alpha or beta amino acid; an alpha or beta peptide; dopamine; a monosaccharide, disaccharide, oligosaccharide or polysaccharide; 1,4-dihydrophenonthrolin-4-one-3-carboxylic acid; polyethylene glycol; and, a therapeutic molecule having a molecular weight of no more than 40,000 Daltons, 30,000 Daltons, 20,000 Daltons, 10,000 Daltons, 5000 Daltons, 3000 Daltons, 2000 Daltons, 1000 Daltons, 500 Daltons, or any range therein in increments of 100 Daltons. Any therapeutic molecule can be used, and in some embodiments, the therapeutic molecule can be selected from the group consisting of an antioxidant, an anti-inflammatory, an analgesic, an antiproliferant, an immunomodulator, an antimicrobial, an antibacterial, an antifungal, an antiviral, or any combination thereof.

Surprisingly, we found that the addition of the electrophilic stabilizer increased the rate of polymerization substantially, and often dramatically, in addition to the function of addressing the problem of depolymerization. It was found that, during water-catalyzed polymerization of dithiolane monomer, for example, we created new capabilities in adhesion through our dithiolane adhesive compositions. We observed a surprisingly rapid transformation of a liquid adhesive precursor into a solid adhesive, for example, which is particularly useful in adhering wet surfaces. Valuable uses include adhering human or animal tissues, as they contribute sufficient water to catalyze an in-situ polymerization of dithiolane monomers upon contact with the wet tissue. As such, the adhesive compositions taught herein, such as the dithiolanes compositions including stabilizer, were particularly useful in adhering devices to tissues, adhering tissues to tissues, and closing puncture wounds in tissues. It was also discovered that the use of NHS esters, NHS being a known electrophile, can increase the formation of covalent bonds with tissue surfaces in the adhesive compositions.

The stability of the polymers in the adhesive compositions can be adjustable, in some embodiments. The choice of stabilizer moieties further allows to design the stability of the polymer, by choosing whether the stabilizer is needed at all for a desired use, and then adjusting the stability of the polymer for it's desired use by choosing a stabilizer moiety having a desired bond strength between the stabilizer and polymer. The same is true of the choice of terminator moieties by choosing whether to the terminator moiety is needed at all for the desired use, and then further adjusting the stability of the polymer choosing a terminator moiety having a desired bond strength between the terminator and polymer.

### Terminators

As noted, there are at least two active thiol ends in the adhesive polymers taught herein, a first active thiol end, and a second active thiol end. The stabilizer bonds to a first active thiol end to help stabilize the polymer from depolymerization and, likewise a terminator can be used to bond with the second active thiol end. In some embodiments, the terms “stabilizer” and “terminator” can be used interchangeably. In some embodiments, the terminator can be an active agent.

It should be appreciated that, in some embodiments, the term “terminator”, “terminator molecule”, “terminating molecule”, “terminator group”, “terminating group”, and the like, can be used interchangeably. In some embodiments, the term “terminator”, “terminator molecule”, “terminating molecule”, “terminator group”, “terminating group”, and the like, can be used interchangeably with the term “stabilizer”, and the terms refer to the a chemical moiety that can bond to the first thiol end and/or second active thiol end. In some embodiments, the bonding of the terminator to the second active thiol group occurs after the bonding of the stabilizer to the first active thiol group. In some embodiments, the stabilizer and the terminator are added concurrently to a reaction mixture and, in some embodiments, the stabilizer and the terminator are added sequentially to the reaction mixture. In some embodiments, the stabilizer is added to a reaction mixture first, and the terminator is added to the reaction mixture second.

In some embodiments, the plurality of terminator molecules having a functional group that forms a first labile bond with the first active thiol end of the polymer. In some embodiments, the plurality of terminator molecules having a functional group that forms a second labile bond with the second active thiol end of the polymer. In some embodiments, the plurality of terminator molecules having a functional group that forms a first and a second labile bond with the first and second active thiol end of the polymer. In some embodiments, each of the plurality of terminator molecules is selected from the group consisting of R′OH, R′CO₂H, and R′SH, where R′ is an alkyl, cycloalkyl, alkenyl, cycloalkenyl, alkynyl, cycloalkynyl, an aryl group having from 1-8 carbons, including smaller versions thereof that include a C1-C6 alkyl group, a C1-C4 alkyl group, a C1-C3 alkyl group, a C2 alkyl group, or a C1 alkyl group.

In some embodiments, the labile bond for the terminator is selected from the group consisting of thioethers, thioesters, and disulfides. The labile bond can be selected to achieve a desired rate of biodegradation, a desired release rate of an active agent, a targeted drug delivery, or a combination thereof.

In some embodiments, the plurality of terminator molecules is selected from the group consisting of alkanols having from 1-8 carbons, alkanoic acids having from 1-8 carbons, and alkylthiols having from 1-8 carbons, and the second labile bond is selected from the group consisting of thioethers, thioesters, and disulfides. Each of these embodiments can include smaller versions thereof that include, for example, a C1-C6 alkyl group, a C1-C4 alkyl group, a C1-C3 alkyl group, a C2 alkyl group, or a C1 alkyl group.

In some embodiments, the plurality of terminator molecules is selected from the group consisting of glycerol, lactic acid, glycolic acid, and citric acid, and any combination thereof.

In some embodiments, the plurality of terminator molecules are biocompatible alcohols, such as those with low acute toxicityxamples of such biocompatible alcohols include ethanol, 1-propanol, 1-butanol, isobutanol, xylitol, erythritol, sorbitol, and malitol.

In some embodiments, the plurality of terminator molecules is selected from the group consisting of ethanol, a substituted 1,2-dithietane with a hydroxyl functionality, a substituted 1,2-dithiolane with a hydroxyl functionality, and a substituted 1,2-dithiane with a hydroxyl functionality.

### Acids

As noted, the polymerization of the monomers into the polymers can be accelerated by adding an acid to the reaction mixture. Any suitable acid can be selected used by the skilled artisan. Examples of desirable acids that can be used include a variety of organic acids, inorganic Bronsted acids, and Lewis acids.

In some embodiments, the acid is a Bronsted acid and can include HCl, H₂SO₄, HNO₃, trifluoroacetic acid, fluoroacetic acid, trichloroacetic acid, acetic acid, citric acid, succinic acid, maleic acid, tartaric acid, gluconic acid, lactic acid, ascorbic acid, limononic acid, toluene sulfonic acid etc.

In some embodiments, the acid is a Lewis acid and can include BCl₃, AlCl₃, BF₃, I₂, Cl₂, ICI, Br₂, transition, alkali earth metal salts, etc.

### The Polymers

The adhesive compositions taught herein include the liquid compositions that create the polymers, the polymers created, and the solid products created using the polymers. As such, it should be appreciated that the instant technology includes at least (i) the adhesive compositions that are used to make the polymers; (ii) the monomers, oligomers, and polymers provided by those compositions; (iii) the products made from those compositions, including liquids, sprays, solid formed articles, and free-flowing powders; and, the methods of (iv) making and (v) using the adhesive compositions, the polymers, the liquids, the sprays, the powders, solid articles formed, and combinations thereof.

The polymers include, of course, medical grade polymers, medical grade polymers having medicinal qualities, and medical grade polymers that include an active agent. In some embodiments, the medical grade polymers include incorporation of an active agent for delivery in a subject, and the delivery can be a targeted delivery to a tissue. In some embodiments, the polymers include the polymers taught above. In some embodiments, the polymer is stabilized with a stabilizer as taught above. In some embodiments, the polymer is stabilized with both a stabilizer and terminator as taught above. In some embodiments, the stabilizer is the same as, or substantially similar to, the monomer used in the polymerization. In some embodiments, the stabilizer is the same as, or substantially similar to, the monomer used in the polymerization, and the terminator is a solvent molecule used in the polymerization process. In some embodiments, the stabilizer is the same as, or substantially similar to, the monomer used in the polymerization, and the terminator is a non-toxic, or relatively non-toxic solvent molecule used in the polymerization process. As such, the instant technology includes stabilized and recyclable polydisulfide polymers as taught herein.

In some embodiments, the polymer has the following structure:

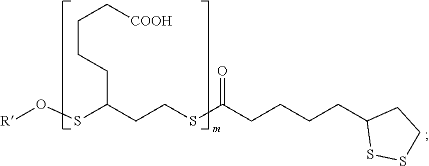

wherein,
m is an integer selected to match the desired molecular weight of the polymer; and,
R′ is an alkyl, cycloalkyl, alkenyl, cycloalkenyl, alkynyl, cycloalkynyl, or aryl group having from 1-8 carbons, 1-6 carbons, 1-4 carbons, 1-3 carbons, 2 carbons, or 1 carbon. In some embodiments R′ and R″ have the same structure as the cyclic disulfide monomer used to form the polymer. In some embodiments, R′ can be an alkyl group, such as an ethyl group.

In some embodiments, the polymer has the following structure:

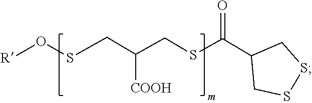

wherein,
m is an integer selected to match the desired molecular weight of the polymer; and,
R′ is an alkyl, cycloalkyl, alkenyl, cycloalkenyl, alkynyl, cycloalkynyl, or aryl group having from 1-8 carbons, 1-6 carbons, 1-4 carbons, 1-3 carbons, 2 carbons, or 1 carbon. In some embodiments R′ and R″ have the same structure as the cyclic disulfide monomer used to form the polymer. In some embodiments, R′ can be an alkyl group, such as an ethyl group.

The mechanical properties of the polymers taught herein can be improved through branching of the polymers. As such, the polymers taught herein include branched forms. For example, in some embodiments, the polymer can have a repeating unit with a substituted dithioalkyl structure as follows:

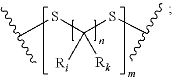

wherein,
n is an integer ranging from 2 to 4;
each Rᵢ and Rₖ is independently selected from the group consisting of H; alkyl, cycloalkyl, alkenyl, alkynyl, and aryl groups having from 1-8 carbons, 1-6 carbons, 1-4 carbons, 1-3 carbons, 2 carbons, or 1 carbon; hydroxylated alkyl, cycloalkyl, alkenyl, alkynyl, and aryl groups having from 1-8 carbons, 1-6 carbons, 1-4 carbons, 1-3 carbons, 2 carbons, or 1 carbon; and, carboxylated alkyl, cycloalkyl, alkenyl, alkynyl, and aryl groups having from 1-8 carbons, 1-6 carbons, 1-4 carbons, 1-3 carbons, 2 carbons, or 1 carbon; wherein, i and k are integers and at least one Rᵢ or Rₖ in each repeating unit includes a carbonyl functionality;
m is an integer selected to match the desired molecular weight of the polymer;
In these embodiments, as in others, the polydisulfide polymer can further have a first active thiol end and a second active thiol end; and, a plurality of stabilizer molecules. The plurality of stabilizer molecules are configured to stabilize the first active thiol end of the polymer, the plurality of stabilizer molecules being the substituted cyclic disulfide molecules derivatized to include a functional group that forms a first labile bond with the first active end, the first labile bond selected from the group consisting of a thioester bond, a thiocarbamate bond, and a thioester bond. And, in these embodiments, the repeating units and the stabilizer molecules can be the same, or substantially the same, chemical moieties following a degradation of the polymer during a recycling of the polymer. When the repeating units and the stabilizer molecules are the same, or substantially the same, chemical moieties following a degradation of the polymer during a recycling of the polymer, the recycling is an easier and more cost-effective commercial process.

Also, such a polymer can be branched by design to increase it's mechanical strength through added features that can include, perhaps, the addition of physical entanglement. In such embodiments, for example, the polymer can be branched from at least one of Rᵢ or Rₖ in a first dithioalkyl repeating unit, the at least one Rᵢ or Rₖ including a carbonyl functionality in a thioester bond with a second dithioalkyl repeating unit. And, in these embodiments, the terminator molecules taught above can be added to further stabilize the polymer from depolymerization.

In some embodiments, the polydisulfide polymer can include the following branched structure

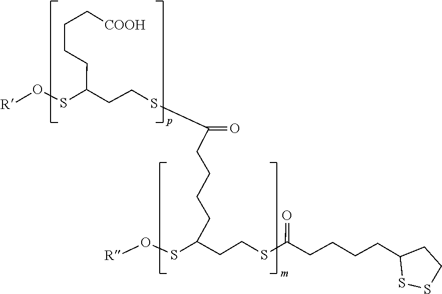

wherein,
m is an integer selected to match the desired molecular weight of the polymer;
p is an integer; and,
R′ and R″ are each independently selected from a group consisting of an alkyl, cycloalkyl, alkenyl, cycloalkenyl, alkynyl, cycloalkynyl, or aryl group having from 1-8 carbons, 1-6 carbons, 1-4 carbons, 1-3 carbons, 2 carbons, or 1 carbon. In some embodiments R′ and R″ have the same structure as the cyclic disulfide monomer used to form the polymer. In some embodiments, R′ and R″ are alkyl groups, such as ethyl groups.

In some embodiments, the polydisulfide polymer can include the following branched structure

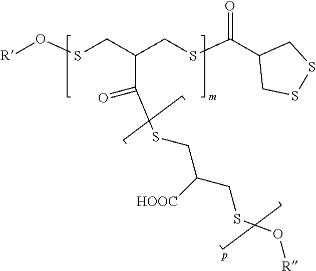

wherein,
m is an integer selected to match the desired molecular weight of the polymer;
p is an integer; and,
R′ and R″ are each independently selected from a group consisting of an alkyl, cycloalkyl, alkenyl, cycloalkenyl, alkynyl, cycloalkynyl, or aryl group having from 1-8 carbons, 1-6 carbons, 1-4 carbons, 1-3 carbons, 2 carbons, or 1 carbon. In some embodiments R′ and R″ have the same structure as the cyclic disulfide monomer used to form the polymer. In some embodiments, R′ and R″ are alkyl groups, such as ethyl groups.

### Methods of Making and Using the Adhesive Compositions

The adhesive compositions can be made in several ways and, for at least this reason, they can be designed for several medical applications. For example, the methods can include placing cyclic disulfide monomers in a polar, protic reaction solvent taught herein; placing cyclic disulfide monomers in a reaction solvent with stabilizer molecules taught herein; and, placing cyclic disulfide monomers in the reaction solvent with the stabilizer molecules and terminator molecules taught herein. In each of these methods of making the adhesive compositions, acid and/or DMSO can be added to speed the rate of reaction, and a conjugate base of the monomers can be added to stabilize the composition. The adhesive compositions can also be dry, in which the polymerization occurs by contacting the dry compositions with water at the target site of the desired adhesion. The adhesive compositions can include precursor polymer, in which the polymer was formed and included as part of the adhesive composition, and polymerization is again initiated with water at the target site of the desired adhesion. The adhesive compositions can formed into solid adhesive articles, including a solid adhesive patch, film, or membrane, and the solid adhesive article can be delivered to the site of adhesion with or without including a liquid, spray, or powder form of the adhesive composition for reaction with water at the target site of the desired adhesion. The adhesive compositions can also be made to include drugs for delivery, including delivery at a target site that can be topical or internal. These and other methods are taught herein.

In some embodiments, the methods include obtaining a plurality of substituted cyclic disulfide molecules having from 2 to 4 C atoms in a cyclic ring with a disulfide bond, the substituted cyclic disulfide molecules functional to polymerize through a ring-opening reaction in polar, protic solvent to form a polymer having
a first active thiol end and a second active thiol end; and,
a repeating unit having a substituted dithioalkyl structure as follows

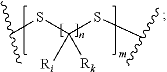

wherein,
   n is an integer ranging from 2 to 4;
   each Rᵢ and Rₖ is independently selected from the group consisting of H; alkyl, cycloalkyl, alkenyl, alkynyl, and aryl groups having from 1-8 carbons, 1-6 carbons, 1-4 carbons, 1-3 carbons, 2 carbons, or 1 carbon; hydroxylated alkyl, cycloalkyl, alkenyl, alkynyl, and aryl groups having from 1-8 carbons, 1-6 carbons, 1-4 carbons, 1-3 carbons, 2 carbons, or 1 carbon; and, carboxylated alkyl, cycloalkyl, alkenyl, alkynyl, and aryl groups having from 1-8 carbons, 1-6 carbons, 1-4 carbons, 1-3 carbons, 2 carbons, or 1 carbon; wherein, i and k are integers and at least one Rᵢ or Rₖ in each repeating unit includes a carbonyl functionality;
   m is an integer selected to match the desired molecular weight of the polymer;
obtaining a plurality of stabilizer molecules configured to stabilize the first active thiol end of the polymer, the plurality of stabilizer molecules being the substituted cyclic disulfide molecules derivatized to include a functional group that forms a first labile bond with the first active end, the first labile bond selected from the group consisting of a thioester bond, a thiocarbamate bond, and a thioester bond; and,
polymerizing the plurality of the substituted cyclic disulfide molecules to create the polymer in the polar, protic solvent.

In some embodiments, the repeating units and the stabilizer molecules are the same, or substantially the same, chemical moieties following a degradation of the polymer. In some embodiments, the methods include adding a bioactive agent to the composition, wherein the bioactive agent is delivered to a target tissue.

The polar, protic solvents used can be any solvent considered suitable by a skilled artisan. Several suitable polar, protic solvents are taught herein, and each can be used at the discretion of the skilled artisan in view of the desired use. In some embodiments, for example, the polar, protic solvent can be pure ethanol, pure water, a combination of ethanol and water, or any aqueous solution suitable for the particular environment. In some embodiments the environment is a medical environment, and the polar, protic solvent includes a saline solution, or a buffer solution that can be used in a living organism. In some embodiments, a bioactive agent can be added to the polar, protic solvent to include with the composition for release, namely for delivery to a target tissue.

In some embodiments, the methods include adding a stabilizer molecule. And, in some embodiments, the methods include adding both a stabilizer molecule and a terminator molecule. Examples of the stabilizer molecules and the terminator molecules that can be used are taught herein, and each can be selected and used at the discretion of the skilled artisan, the stabilizer used alone or in combination with a terminator, the stabilizer or the stabilizer and terminator combination selected and used as a particular polymer configuration designed in view of a desired application. In some embodiments, the stabilizer and/or the terminator molecules can include a bioactive agent added to the composition for release, namely for delivery to a target tissue.

The polymerization reaction and stabilization reactions can be concurrent or in series. In some embodiments, the method can include placing the monomer and the stabilizer together in a reaction mixture and reacting them together. In some embodiments, the monomer, the stabilizer, and/or the terminator molecules can include a bioactive agent added to the composition for release, namely for delivery to a target tissue.

In some embodiments, the method can include polymerizing the monomer first, and then adding the stabilizer as a separate reaction. In some embodiments, the method can include placing the monomer, the stabilizer, and the terminator together in a reaction mixture and reacting them together. In some embodiments, the method can include polymerizing the monomer first, and then adding the stabilizer and terminator for a separate reaction. In some embodiments, the method can include polymerizing the monomer first, and then adding the stabilizer for a second reaction, and then adding the terminator for a third reaction. A bioactive agent can be added to polymer produced using ionic binding, covalent bonding, hydrogen bonding, mechanical entrapment, or any combination thereof, in some embodiments.

The polymerization reaction and stabilization reactions can be concurrent or in series, with the reactant amounts controlled to drive the reactions and control competing reactions. In some embodiments, the methods include adding an amount of monomer that corresponds to m, and allowing the monomer to react to depletion in a first reaction, and then adding an amount of stabilizer and terminator for stabilizing the polymer, the amount of stabilizer and terminator corresponding to the expected amounts of first active thiol and second active thiol for a second reaction. In some embodiments, the methods include adding an amount of monomer that corresponds to m, and allowing the monomer to react to depletion in a first reaction, adding an amount of stabilizer that corresponds to an expected amount of first active thiol for a second reaction, and adding an amount of terminator that corresponds to an expected amount of second active thiol for a third reaction.

The polymerization reactions taught herein can be accelerated by adding acid and/or DMSO. In some embodiments, the methods further include adding DMSO to a mixture of cyclic disulfide monomers to accelerate the polymerization reaction. In some embodiments, the methods further include adding an acid taught herein to a mixture of cyclic disulfide monomers to accelerate the polymerization reaction. In some embodiments, the methods further include adding DMSO and acid taught herein to a mixture of cyclic disulfide monomers to accelerate polymerization the reaction. In some embodiments, the mixture of cyclic disulfide monomers can include stabilizer. In some embodiments, the mixture of cyclic disulfide monomers can include stabilizer and terminator.

The adhesive compositions can be concentrated as stable concentrated adhesive compositions that polymerize at the target site of adhesion. In some embodiments, the cyclic disulfide monomer can be combined with a stabilizer in a desired polar, protic solvent as taught herein. Vacuum is applied to the mixture for removal of most of the solvent, such that the mixture is a concentrated monomer and stabilizer in a small amount as a residual solvent. The concentrated adhesive composition can be applied to wet surfaces to initiate polymerization at the target site of adhesion.

The adhesive compositions can be coated onto a first surface for use as a pressure sensitive adhesive (PSA) to adhere the first surface to a second surface. A liquid form of an adhesive composition, included the concentrated form, can be applied to the first surface. The solvent in the adhesive composition can be allowed to evaporate, be drawn off under reduced pressure, or heated to produce a thin film adhesive on the first surface for adhering the first surface to the second surface. The adhesion can be reversible or irreversible and accomplished by making contact between the first surface and the second surface. The contact can include use of a modest pressure, such as the use of finger or hand pressure to the contact made between the first surface and the second surface.

A solid adhesive article can be formed with the adhesive compositions and used as an adhesive in itself. In some embodiments, the solid adhesive article can be an adhesive patch formed from the adhesive compositions. In some embodiments, the solid article can be a coating that has been applied as a coating on a surface, and the stickiness of the exposed surface of the coating is removed by blocking the surface of the solid adhesive coating. In some embodiments, for example, a telechelic PEG polymer can be functionalized with α-lipoic acid and sprayed on top of a newly formed adhesive coating layer. Such coatings can also be used to carry active agents, deliver active agents, and the like.

The solid adhesive article can be formed using molding, casting, or extrusion, for example. In some embodiments, the solid adhesive article can be an adhesive film. In some embodiments, the solid adhesive article can be an adhesive membrane. Any liquid, spray, or concentrated adhesive composition taught herein can be used in the formation of the solid adhesive article. In some embodiments, a polar, protic solvent such as water or a water containing solvent or buffer can be added to any adhesive composition taught herein to obtain a wet adhesive polymer composition. The wet adhesive polymer composition can be formed into the solid adhesive article. In some embodiments, the solid adhesive article may be self-supporting or supported on a temporary backing material. The article can then be adhered to a surface.

The adhesive compositions taught herein can be thermally polymerized to form a solid adhesive article. In some embodiments, the adhesive composition is heated to thermally polymerize the composition to create a bulk polymer that can be formed into a solid adhesive article. The solid adhesive article can be formed using molding, casting, or extrusion, for example. In some embodiments, the solid adhesive article can be an adhesive film. In some embodiments, the solid adhesive article can be an adhesive membrane. In some embodiments, the solid adhesive article may be self-supporting or supported on a temporary backing material. The article can then be adhered to a surface. A modest hand or finger pressure can be used as with the pressure sensitive adhesives to form an adhesion between the article and the surface.

A modest hand or finger pressure can be used as with the adhesives to form an adhesion between the article and the surface. In some embodiments, a powder, liquid, or combination thereof can be applied to the surface of the solid adhesive article before adhering the solid adhesive article to a surface. In some embodiments, the powder, liquid, or combination thereof can be applied as a spray form of the adhesive compositions which can be applied to the surface of the solid adhesive article before adhering the solid adhesive article to the surface.

The liquid adhesive compositions can be concentrated by removing solvent to initiate polymerization and create an adhesive composition that contains sticky polymer in solution. The liquid adhesive composition can be referred to as a “liquid precursor” comprising monomer, monomer and stabilizer, or monomer and stabilizer and terminator, and the concentrated and polymerized composition can be referred to as a “polymer precursor”, or “macromolecular precursor”. The concentrated and polymerized composition can be made by allowing solvent to evaporate, using reduced pressure, or by heating, for example. The polymer precursor can be rediluted as a polymer precursor solution and used as the adhesive composition. The adhesive composition having the polymer precursor can be used to bond surfaces together, preferably in the presence of water to further polymerization and strengthen the bond.

The liquid adhesive compositions can be concentrated by removing solvent to initiate polymerization and create an adhesive composition that contains sticky polymer in solution, and then the polymer can be dried for later use. The liquid adhesive composition can be referred to as a “liquid precursor” comprising monomer, monomer and stabilizer, or monomer and stabilizer and terminator, and the concentrated and polymerized composition can be referred to as a “polymer precursor”, or “macromolecular precursor”. The concentrated and polymerized composition can be made by allowing solvent to evaporate, using reduced pressure, or by heating, for example. The polymer precursor can be dried for later use as a dry polymer used as an adhesive composition, or rediluted as a polymer precursor solution and used as the adhesive composition. The adhesive composition having the polymer precursor can be used to bond surfaces together, both dry and wet compositions preferably in the presence of water to further polymerization and strengthen the bond. The dried polymer can be formed into a solid adhesive article.

The methods can include any combination selected from the group consisting of a monomer as taught herein, a stabilizer as taught herein, a reaction solvent as taught herein, DMSO, and an acid as taught herein including Lewis acids and Bronsted acids. In some embodiments, the methods of making the adhesive composition include combining a cyclic disulfide monomer with a stabilizer in as a solid composition, wherein the solid composition can be polymerized at a desired time by contacting the solid composition with a reaction solvent, or by thermal polymerization. In some embodiments, the methods of making the adhesive composition include combining a cyclic disulfide monomer with a stabilizer and a reaction solvent. In some embodiments, the methods of making the adhesive composition include combining a cyclic disulfide monomer with a stabilizer and DMSO. In some embodiments, the methods of making the adhesive composition include combining a cyclic disulfide monomer with a stabilizer and an acid. In some embodiments, the methods of making the adhesive composition include combining a cyclic disulfide monomer with a stabilizer and a reaction solvent including DMSO. In some embodiments, the methods of making the adhesive composition include combining a cyclic disulfide monomer with a stabilizer and a reaction solvent including DMSO and an acid. Cross-linkers can be included, as taught herein, in any of these methods to at least further strengthen the adhesives. It should be appreciated that the reaction solvent can provide the terminator in some embodiments. For example, ethanol and ethanol/water mixtures work well as solvents, and the ethanol works well as a terminator. As such, the terminator can be easily provided through a careful selection of the reaction solvent.

The methods can include any combination of components selected from the group consisting of a polydisulfide polymer as taught herein, a monomer as taught herein, a stabilizer as taught herein, a reaction solvent as taught herein, DMSO, and an acid as taught herein including Lewis acids and Bronsted acids. In some embodiments, the combination of components includes one or more bioactive agents. In some embodiments, the methods of making the adhesive composition include a polydisulfide polymer as a solid composition, wherein the solid composition can be polymerized at a desired time by contacting the solid composition with a reaction solvent, or by thermal polymerization. In some embodiments, the methods of making the adhesive composition include combining a polydisulfide polymer with an acid as a solid composition, wherein the solid composition can be polymerized at a desired time by contacting the solid composition with a reaction solvent, or by thermal polymerization. In some embodiments, the methods of making the adhesive composition include combining a polydisulfide polymer with a cyclic disulfide monomer and a stabilizer in as a solid composition, wherein the solid composition can be polymerized at a desired time by contacting the solid composition with a reaction solvent, or by thermal polymerization. In some embodiments, the methods of making the adhesive composition include combining a polydisulfide polymer and a cyclic disulfide monomer with a stabilizer and a reaction solvent.

In some embodiments, the methods of making the adhesive composition include combining a polydisulfide polymer and a cyclic disulfide monomer with a stabilizer and DMSO. In some embodiments, the methods of making the adhesive composition include combining a polydisulfide polymer and a cyclic disulfide monomer with a stabilizer and an acid. In some embodiments, the methods of making the adhesive composition include combining a polydisulfide polymer and a cyclic disulfide monomer with a stabilizer and a reaction solvent including DMSO. In some embodiments, the methods of making the adhesive composition include combining a polydisulfide polymer and a cyclic disulfide monomer with a stabilizer and a reaction solvent including DMSO and an acid. The conjugate base of the monomers can be included, as taught herein, to at least provide stability to the composition before polymerization. Terminators can be included, as taught herein, in any of these methods to at least further stabilizer the polymers. Cross-linkers can be included, as taught herein, in any of these methods to at least further strengthen the adhesives. It should be appreciated that the reaction solvent can provide the terminator in some embodiments. For example, ethanol and ethanol/water mixtures work well as solvents, and the ethanol works well as a terminator. As such, the terminator can be easily provided through a careful selection of the reaction solvent. And, any of the embodiments can include the addition of one or more bioactive agents to the compositions.

An increase in the reactant concentration causes a decrease in polymerization time. In some embodiments, the reactants are monomers, or monomers and stabilizers, and the concentration ranges from 100 mg/ml to 1000 mg/ml, from 100 mg/ml to 900 mg/ml, from 100 mg/ml to 800 mg/ml, from 100 mg/ml to 700 mg/ml, from 100 mg/ml to 600 mg/ml, from 100 mg/ml to 500 mg/ml, from 100 mg/ml to 400 mg/ml, from 100 mg/ml to 300 mg/ml, from 100 mg/ml to 200 mg/ml, 200 mg/ml to 1000 mg/ml, from 200 mg/ml to 900 mg/ml, from 200 mg/ml to 800 mg/ml, from 200 mg/ml to 700 mg/ml, from 200 mg/ml to 600 mg/ml, from 200 mg/ml to 500 mg/ml, from 200 mg/ml to 400 mg/ml, from 200 mg/ml to 300 mg/ml, 300 mg/ml to 1000 mg/ml, from 300 mg/ml to 900 mg/ml, from 300 mg/ml to 800 mg/ml, from 300 mg/ml to 700 mg/ml, from 300 mg/ml to 600 mg/ml, from 300 mg/ml to 500 mg/ml, from 300 mg/ml to 400 mg/ml, 400 mg/ml to 1000 mg/ml, from 400 mg/ml to 900 mg/ml, from 400 mg/ml to 800 mg/ml, from 400 mg/ml to 700 mg/ml, from 400 mg/ml to 600 mg/ml, from 400 mg/ml to 500 mg/ml, 500 mg/ml to 1000 mg/ml, from 500 mg/ml to 900 mg/ml, from 500 mg/ml to 800 mg/ml, from 500 mg/ml to 700 mg/ml, from 500 mg/ml to 600 mg/ml, or any amount or range therein in increments of 10 mg/ml. In some embodiments, the reactants are monomers, or monomers and stabilizers, and the concentration is about 100 mg/ml, 200 mg/ml, 300 mg/ml, 400 mg/ml, 500 mg/ml, 600 mg/ml, 700 mg/ml, 800 mg/ml, 900 mg/ml, 1000 mg/ml, 1100 mg/ml, 1200 mg/ml, 1300 mg/ml, 1400 mg/ml, 1500 mg/ml, 1600 mg/ml, 1700 mg/ml, 1800 mg/ml, 1900 mg/ml, 2000 mg/ml, or any amount or range therein in increments of 10 mg/ml.

In some embodiments, the term “about” can be used to refer to a range of amounts that surround the quantity that is modified by the term. It should be appreciated that one amount is “about” the same as another amount when the effects of the change of the amount do not make an appreciable change in the yield, function, activity, toxicity, or any other significant characteristic of a process or product produced by the process. The term “about” is often used to cover such ranges, and it is intended herein to be construed to include amounts that do not have such a range at all; and, therefore, the term “about” is intended to be in amending patent claims to resolve, for example, any concerns presented about indefiniteness of such an amount, or written description, or perhaps even enablement. In some embodiments, when removing the term “about”, the doctrine of equivalents can be assumed when construing the claims.

In some embodiments, the reactants are monomers, or monomers and stabilizers, and the concentration in solution is maximized by evaporating solvent to only a residual amount by evaporating, reducing pressure, or heating the reaction mixture to an observable minimal amount of solvent. In some embodiments, the minimal amount of solvent is sufficient to allow the monomers to remain soluble until polymerization. In some embodiments, the minimal amount of solvent is only that which allows the solvent to be observed within a polymerized monomer in the reaction mixture. In some embodiments, the minimal amount of solvent is that in which the solvent appears entirely removed but shows a substantial presence within a polymerized monomer in a chemical analysis of the reaction mixture.

The amounts of the reaction components can be expressed in mol %. As noted, the composition of the reaction mixtures can be all monomer; monomer and stabilizer; monomer, stabilizer, and terminator; or, monomer, stabilizer, and terminator. DMSO, an acid taught herein, or a combination of DMSO and an acid can be added to increase the rate of polymerization in the reaction mixtures. Cross-linkers can be added to increase the strength of the adhesive compositions.

In some embodiments, the mol % of the monomer (or combination of monomer and it's conjugate base) can range from 80 mol % to 100 mol %, from 80 mol % to 95 mol %, from 80 mol % to 90 mol %, from 80 mol % to 85 mol %, or any range or mol % therein in increments of 1 mol %, such as about 80 mol %, about 81 mol %, about 82 mol %, about 83 mol %, about 84 mol %, about 85 mol %, about 86 mol %, about 87 mol %, about 88 mol %, about 89 mol %, about 90 mol %, about 91 mol %, about 92 mol %, about 93 mol %, about 94 mol %, about 95 mol %, about 96 mol %, about 97 mol %, about 98 mol %, about 99 mol %, about 100 mol %. In embodiments that combine the monomer with it's conjugate base, the combination of the monomer to the conjugate base can be at a ratio that ranges from 5:1 to 1:5, such as 1:1, 2:1, 3:1, 4:1, or 5:1, 1:5, 1:4, 1:3, or 1:2 in the total mol % of that component.

In some embodiments, the mol % of the stabilizer can range from 0 mol % to 20 mol %, 0 mol % to 15 mol %, 0 mol % to 10 mol %, 0 mol % to 5 mol %, or any range or mol % therein in increments of 1 mol %, such as about 0 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, and about 20 mol %, or any amount or range therein in increments of 0.5 mol %. This same amount of mol % can be used for the terminator, as the ratio of first active thiol to second active thiol can reasonably be assumed to be the same or similar in embodiments taught herein. However, it should be appreciated that, since alkanols work well for the terminator, and since ethanol is a convenient reaction solvent or component of a reaction solvent in some embodiments, the reaction solvent can serve as a source of excess terminator in such embodiments.

In some embodiments, the mol % of the bioactive agent can range from 0.01 mol % to 20 mol %, 0.01 mol % to 15 mol %, 0.01 mol % to 10 mol %, 0.01 mol % to 5 mol %, or any range or mol % therein in increments of 0.01 mol %, such as about 0.01 mol %, about 0.10 mol %, about 0.20 mol %, about 0.30 mol %, about 0.40 mol %, about 0.50 mol %, about 0.60 mol %, about 0.70 mol %, about 0.80 mol %, about 0.90 mol %, about 1.0 mol %, about 2.0 mol %, about 3.0 mol %, about 4.0 mol %, about 5.0 mol %, about 6.0 mol %, about 7.0 mol %, about 8.0 mol %, about 9.0 mol %, about 10 mol %, about 11 mol %, about 12 mol %, about 13 mol %, about 14 mol %, about 15 mol %, about 16 mol %, about 17 mol %, about 18 mol %, about 19 mol %, and about 20 mol %, or any amount or range therein in increments of 0.01 mol %.

Poly(asparagusic acid) polymerization is an example of a method taught herein that results in a polymer that may not require a stabilizer, at least in some uses of the adhesive. The same application of poly(α-lipoic acid), however, would require a stabilizer due to a lower ring strain in the α-lipoic acid monomer. Due to the higher ring strain of asparagusic acid, the poly(asparagusic acid) tends to be slower to depolymerize relative to poly(α-lipoic acid). That said, the use of a stabilizer can be used to decrease the rate of depolymerization of any polymer taught herein, and the need for such added stability depends on the intended use of the polymer.

In some embodiments, DMSO is not used to catalyze the reactions, and in embodiments in which DMSO is used to help facilitate polymerization, the DMSO can be added in what a skilled artisan would consider “a catalytic amount”, which is readily determined by the skilled artisan. In some embodiments, the DMSO is added to a reaction mixture in an amount ranging from 0 mol % to the catalytic amount. In some embodiments, the DMSO is added to a reaction mixture in an amount ranging from 0 ul/ml to 500 ul/ml, from 50 ul/ml to 400 ul/ml, from 75 ul/ml to 300 ul/ml, from 100 ul/ml to 200 ul/ml, or any range or amount therein in increments of 10 ul/ml. In some embodiments, the DMSO is added to a reaction mixture in an amount of about 50 ul/ml, about 75 ul/ml, about 100 ul/ml, about 125 ul/ml, about 150 ul/ml, about 175 ul/ml, about 200 ul/ml, about 225 ul/ml, about 250 ul/ml, about 275 ul/ml, about 300 ul/ml, or any range or amount therein in increments of 5 ul/ml.

In some embodiments, acid is not used to catalyze the reactions, and in embodiments in which acid is used to help facilitate polymerization, the acid can be added in what a skilled artisan would consider “a catalytic amount”, which is readily determined by the skilled artisan. In some embodiments, the acid is added to a reaction mixture in an amount ranging from 0 mol % to the catalytic amount. In some embodiments, the acid is added to a reaction mixture in an amount ranging from 0 mol % to 20 mol %, from 5 mol % to 20 mol %, from 10 mol % to 20 mol %, from 15 mol % to 20 mol %, or any range or amount therein in increments of 1 mol %. In some embodiments, the acid is added in an amount of about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, or any range or amount therein in increments of 0.5 mol %.

As noted herein, a cross-linker can be added to increase the strength of the adhesions compositions. It should be appreciated that any cross-linker known to those of skill in the art of polymer chemistry and materials can be used where suitable and appropriate to the intended use and the chemical structure and functionality of the adhesive composition. In some embodiments, the process includes adding a cross-linker taught herein. The type of cross-linker selected can affect the amount of cross-linker needed, as can the type of cross-linker selected, and the amount added, can be chosen for a particular desired use of the adhesive composition. For example, a cross-linker can be added to create a desired mechanical response of the adhesive composition and/or, perhaps, a desired thermal response of the adhesive composition and/or, perhaps a desired thermomechanical response of the adhesive composition. In some embodiments, a mechanical response may be the “softness” of the composition, and the cross-linker may be added to modulate the polymer to have a lower modulus and higher fracture strain where a more “stretchable” adhesive composition may be desired. In another example, strength and hardness may be more desirable where the environment can be more punishing, such as in thin-film solar cells integrated with rooftops, roads, sidewalks, parking lots, and vehicle and aeronautic surfaces; heads-up displays in eyeglasses, windshields, and cockpits; and, perhaps integration with textiles, especially in physically demanding contexts (military, rescue, and medical workers). One of skill will appreciate that any of a variety of cross-linkers, including those taught herein, can be added to obtain any of a variety of mechanical responses in the adhesive compositions, with the desired mechanical response depends on the desired use for the adhesive composition.

In some embodiments, a crosslinker can be added in an amount of 1 mol % to 40 mol %, 1 mol % to 35 mol %, 1 mol % to 30 mol %, 1 mol % to 25 mol %, 1 mol % to 20 mol %, 1 mol % to 15 mol %, 1 mol % to 10 mol %, 1 mol % to 5 mol %, 5 mol % to 40 mol %, 5 mol % to 35 mol %, 5 mol % to 30 mol %, 5 mol % to 25 mol %, 5 mol % to 20 mol %, 5 mol % to 15 mol %, 5 mol % to 10 mol %, 10 mol % to 40 mol %, 10 mol % to 35 mol %, 10 mol % to 30 mol %, 10 mol % to 25 mol %, 10 mol % to 20 mol %, 10 mol % to 15 mol %, or any amount or range therein in increments of 1 mol %. In some embodiments, the cross-linker can be added in an amount of about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, or any amount, range, or combination of these amounts.

The pH of the reactions can also be varied to achieve desired chemical activity in the reaction, namely reaction kinetics and products. In the polymerization reactions taught herein, for example, it was found that a lower pH can be expected to enhance polymerization. The pH that works the best for a set of reaction conditions will depend on the monomeric components being polymerized, the presence and type of reaction solvent, and the pressure of the reaction. In some embodiments, for example, the pH can range from a pH of 1 to a pH of 9, from a pH of 1 to a pH of 8, from a pH of 1 to a pH of 7, from a pH of 1 to a pH of 6, from a pH of 1 to a pH of 5, from a pH of 1 to a pH of 4, from a pH of 1 to a pH of 3, from a pH of 1 to a pH of 2, from a pH of 2 to a pH of 9, from a pH of 2 to a pH of 8, from a pH of 2 to a pH of 7, from a pH of 2 to a pH of 6, from a pH of 2 to a pH of 5, from a pH of 2 to a pH of 4, from a pH of 2 to a pH of 3, from a pH of 3 to a pH of 9, from a pH of 3 to a pH of 8, from a pH of 3 to a pH of 7, from a pH of 3 to a pH of 6, from a pH of 3 to a pH of 5, from a pH of 3 to a pH of 4, from a pH of 4 to a pH of 9, from a pH of 4 to a pH of 8, from a pH of 4 to a pH of 7, from a pH of 4 to a pH of 6, from a pH of 4 to a pH of 5, from a pH of 5 to a pH of 9, from a pH of 5 to a pH of 8, from a pH of 5 to a pH of 7, from a pH of 5 to a pH of 6, from a pH of 6 to a pH of 9, from a pH of 6 to a pH of 8, from a pH of 6 to a pH of 7, from a pH of 6 to a pH of 8, from a pH of 6 to a pH of 7, from a pH of 7 to a pH of 8, or any pH or pH range therein in increments of 0.1 pH. In some embodiments, the pH can be a pH of about 1, a pH of about 2, a pH of about 3, a pH of about 4, a pH of about 5, a pH of about 6, a pH of about 7, a pH of about 8, a pH of about 9, or any pH or pH range therein in increments of 0.1 pH. The pH can also vary during the course of a reaction, in some embodiments. The effect can be to increase the kinetics of the reaction, decrease the kinetics of the reaction, quell the reaction, or convert the reaction products. In some embodiments, the pH can be increased during the course of such a process. In some embodiments, the pH can be decreased during the course of such a process. And, in some embodiments the pH can oscillate during the course of such a process.

The temperature of the reactions can also be varied to achieve desired chemical activity in the reaction, namely reaction kinetics and products. In the polymerization reactions taught herein, for example, a lower temperature can be expected to at least reduce the rate of polymerization, and a higher temperature can be expected to at least increase the rate of polymerization. Likewise, the rate of depolymerization will also decrease at a lower temperature and increase at a higher temperature. It should be appreciated that, if we increase the temperature, then we have more product and the reaction shifts towards the reactants; and, if we decrease the temperature, then have less product and so the reaction shifts towards the products. The temperature that works the best for a set of reaction conditions will depend on the monomeric components being polymerized, the presence and type of reaction solvent, and the pressure of the reaction. In some embodiments, for example, the temperature can range from 0° C. to 120° C., from 5° C. to 120° C., from 10° C. to 120° C., from 15° C. to 120° C., from 20° C. to 120° C., from 25° C. to 120° C., from 30° C. to 120° C., from 35° C. to 120° C., from 40° C. to 120° C., from 45° C. to 120° C., from 50° C. to 120° C., from 55° C. to 120° C., from 60° C. to 120° C., from 65° C. to 120° C., from 70° C. to 120° C., from 75° C. to 120° C., from 80° C. to 120° C., from 85° C. to 120° C., from 90° C. to 120° C., from 95° C. to 120° C., from 100° C. to 120° C., from 105° C. to 120° C., from 110° C. to 120° C., from 115° C. to 120° C., or any amount or range therein in increments of 1° C. In some embodiments, for example, the temperature can range from 10° C. to 100° C., from 15° C. to 100° C., from 20° C. to 100° C., from 25° C. to 100° C., from 30° C. to 100° C., from 35° C. to 100° C., from 40° C. to 100° C., from 45° C. to 100° C., from 50° C. to 100° C., from 55° C. to 100° C., from 60° C. to 100° C., from 65° C. to 100° C., from 70° C. to 100° C., from 75° C. to 100° C., from 80° C. to 100° C., from 85° C. to 100° C., from 90° C. to 100° C., from 95° C. to 100° C., or any amount or range therein in increments of 1° C. In some embodiments, for example, the temperature can range from 20° C. to 100° C., from 25° C. to 100° C., from 30° C. to 100° C., from 35° C. to 100° C., from 40° C. to 100° C., from 45° C. to 100° C., from 50° C. to 100° C., from 55° C. to 100° C., from 60° C. to 100° C., from 65° C. to 100° C., from 70° C. to 100° C., from 75° C. to 100° C., from 80° C. to 100° C., from 85° C. to 100° C., from 90° C. to 100° C., from 95° C. to 100° C., or any amount or range therein in increments of 1° C. In some embodiments, for example, the temperature can range from 20° C. to 80° C., from 25° C. to 80° C., from 30° C. to 80° C., from 35° C. to 80° C., from 40° C. to 80° C., from 45° C. to 80° C., from 50° C. to 80° C., from 55° C. to 80° C., from 60° C. to 80° C., from 65° C. to 80° C., from 70° C. to 80° C., from 75° C. to 80° C., or any amount or range therein in increments of 1° C. In some embodiments, for example, the temperature can range from 20° C. to 60° C., from 25° C. to 60° C., from 30° C. to 60° C., from 35° C. to 60° C., from 40° C. to 60° C., from 45° C. to 60° C., from 50° C. to 60° C., from 55° C. to 60° C., from 60° C. to 60° C., or any amount or range therein in increments of 1° C. In some embodiments, for example, the temperature can range from 20° C. to 40° C., from 25° C. to 40° C., from 30° C. to 40° C., from 35° C. to 40° C., or any amount or range therein in increments of 1° C. In some embodiments, for example, the temperature can be about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., 52° C., about 53° C., 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., or any amount or range therein in increments of 0.1° C. Temperature can also vary during the course of a reaction, in some embodiments. The effect can be to increase the kinetics of the reaction, decrease the kinetics of the reaction, quell the reaction, or convert the reaction products. In some embodiments, the temperature can be increased during the course of such a process. In some embodiments, the temperature can be decreased during the course of such a process. And, in some embodiments the temperature can be varied one or more times during the course of a polymerization, or oscillate during the course of such a process.

The pressure of the reactions can also be varied. The reactions can be in solution, suspension, or a solid, in a reaction solvent that can include any reaction solvent taught herein, for example, polar, protic solvents, polar, aprotic solvents, alcohol solvents, aqueous solvents, water, any combination of those, and the like. The amount of pressure desired can vary according to the reaction solvent, or solvent combination, selected for the reaction. In some embodiments, the pressure can vary from 1 bar to a pressure reduced as low as 0 bar. In some embodiments, the pressure is limited to the limits of vacuum source used including, for example, about 10⁻¹ torr, about 10⁻² torr, about 10⁻³ torr, about 10⁻⁴ torr, about 10⁻⁵ torr, about 10⁻⁶ torr, about 10⁻⁷ torr, about 10⁻⁸ torr, about 10⁻⁹ torr, about 10⁻¹⁰ torr, about 10⁻¹¹ torr, or any amount or range therein. In some embodiments, high pressures can be used for reactions during the formation of a a desired solid product from polymeric materials in a compression process, and the pressure used can depend on the temperature used, where the pressure increases as the temperature decreases, and the pressure decreases and the temperature increases. In some embodiments, the pressure used can range from 14.7 psi to 10,000 psi, or any amount or range therein in increments of 1 psi. In some embodiments, the pressure used can range from 100 psi to 10,000 psi, 200 psi to 10,000 psi, from 300 psi to 10,000 psi, from 400 psi to 10,000 psi, from 500 psi to 10,000 psi, from 600 psi to 10,000 psi, from 700 psi to 10,000 psi, from 800 psi to 10,000 psi, from 900 psi to 10,000 psi, from 1000 psi to 10,000 psi, from 2000 psi to 10,000 psi, from 3000 psi to 10,000 psi, from 4000 psi to 10,000 psi, from 5000 psi to 10,000 psi, from 6000 psi to 10,000 psi, from 7000 psi to 10,000 psi, from 8000 psi to 10,000 psi, from 9000 psi to 10,000 psi, from 2000 psi to 5,000 psi, from 5000 psi to 10,000 psi, or any amount or range therein in increments of 10 psi. In some embodiments, the pressure used can be about 100 psi, about 500 psi, about 1000 psi, about 1500 psi, about 2000 psi, about 2500 psi, about 3000 psi, about 3500 psi, about 4000 psi, about 4500 psi, about 5000 psi, about 5500 psi, about 6000 psi, about 6500 psi, about 7000 psi, about 7500 psi, about 8000 psi, about 8500 psi, about 9000 psi, about 9500 psi, 10,000 psi, or any amount or range therein in increments of 10 psi. Pressure can also vary during the course of a reaction, in some embodiments. The effect can be to increase the kinetics of the reaction, decrease the kinetics of the reaction, quell the reaction, or convert the reaction products. In some embodiments, the pressure can be increased during the course of such a process. In some embodiments, the pressure can be decreased during the course of such a process. And, in some embodiments the pressure can oscillate during the course of such a process.

Any combination of reaction conditions set-forth herein can be selected to obtain the process and products desired, and it should be appreciated that these conditions, excepting the occurrence of conditions that produce surprisingly desirable results, can be readily determined by the skilled artisan without undue experimentation. Several examples of actual reaction conditions and results have been provided herein to enable the skilled artisan in the practice of the invention.

Moreover, any of the adhesive compositions, or solid adhesive articles formed from the compositions, can be sterilized using pure ethanol, a water-ethanol mixture, UV light, gamma irradiation, ethylene oxide or a combination thereof.

### Adhesive Composition Products

The adhesive composition can take several forms, and can serve as an adhesive, a sealant, and/or a hemostat, and each of these can also carry one or more drugs and, accordingly, be used to deliver the one or more drugs. The compositions can be, for example, a liquid, a solid, a liquid and a solid, and a free-flowing solid which can include powders. The liquid compositions can be directly sprayed or brushed onto surfaces for bonding, or onto a solid form of the adhesive, such as a patch, film, or membrane formed from the polymer. The solid, such as the patch, film, or membrane can be configured for direct application as an adhesive. In some embodiments, the adhesive composition functions as an adhesive alone. In some embodiments, the adhesive composition functions as a sealant. In some embodiments, the adhesive composition functions as a hemostat. In some embodiments, the adhesive composition functions as a therapeutic agent. In some embodiments, the adhesive composition functions as any combination of adhesive, sealant, hemostat, and therapeutic agent.

In medical applications, the adhesive compositions are biocompatible and exhibit many of the features of superglue, such as superfast polymerization and high adhesion strength to tissue. The adhesive compositions can be used in the form of a spray, brush, or solid adhesive article for various applications. The excellent biocompatibility and biodegradability of the adhesive compositions allow for both topical use and internal uses of the adhesive compositions in medical treatments. In some embodiments, the adhesive compositions can also function as a barrier to the transfer of microbials, such as bacteria, making the adhesive compositions superior to hydrogel-based adhesives currently approved for internal applications. Like all of the adhesive compositions taught herein, the products that are PSAs and structural adhesives show excellent underwater adhesion.

### Medical Uses

As taught herein, the adhesive compositions can be made to function very well as medical adhesives, sealants, and hemostats. The products include powder adhesives, liquid adhesives; spray adhesives; solid adhesive articles such as adhesive patches, films, and membranes; and, free-flowing solids such as powders. Combinations of these types of products are also provide, for example, a solid article decorated with a powder, a solid article decorated with a liquid, and the like.

The medical products can be used as a topical tissue adhesive, topical tissue sealant, or a topical tissue adhesive and sealant. These products can be used, for example, in closure of topical wounds created by surgical incisions or traumatic injuries. These products can be applied to any such topical use.

The medical products can be used as an internal tissue adhesive, an internal tissue sealant, an internal tissue hemostat, or any combination thereof. These products can be used, for example, in closure of internal wounds created by surgical incisions or traumatic injuries. These products can be applied to any such internal use. In some embodiments, the use can be for adhesion during a surgical procedure. Examples include any tissue applications including, but not limited to, cartilage repair, tendon repair, ligament repair, anastomosis of blood vessels or intestines, vascular graft attachment, sealing of intestine or stomach or lung or bladder punctures, general hemostasis, spinal dural sealant, ocular sealant, dural sealant, lung parenchymal sealant, gastric perforation sealant, fetal membrane sealant, etc. In some embodiments, these medical products can be used in circumcisions, implant grafting, plastic surgeries, and the like.

The medical products can be used as an adjunct to other existing procedures as an adhesive, sealant, hemostat, a drug delivery composition or device, or any combination thereof. The proven function of the adhesive compositions as products for adhesion, sealant, and as a hemostat, allows the products to be used with any existing medical procedure, thus improving those procedures. In some embodiments, the adhesive products taught herein can be used as an adjunct material for suturing, stapling, tacks, and the like. In some embodiments, the adhesive products taught herein can be used as an adjunct in the anchoring of medical devices to tissue, the attachment of wound dressings, the attachment of skin grafts, the attachment of collagen membranes, as well as with decellularized tissues, ostomy devices, and other synthetic medical devices. The delivery of one or more bioactive agents can be used as an application in itself, or to facilitate most any medical use of the compositions.

### Drug Delivery

It should be appreciated that, in some embodiments, the adhesive compositions are entirely non-hydrogel. As such, non-hydrogel adhesives are provided, and non-hydrogel drug delivery systems are provided herein, in some embodiments. One or more bioactive agents can be delivered to one or more tissues topically, internally, or a combination thereof. In some embodiments, the one or more active agents can be selected from the group consisting of an antioxidant, an antibiotic, an anti-inflammatory, an analgesic, an antiproliferant, an anticancer, an immunomodulator, an antimicrobial, an antibacterial, an antifungal, an antiviral, and any combination thereof. In some embodiments, the one or more active agents can be a small molecule; a biologic that can include a component selected from the group consisting of an amino acid sequence or a nucleic acid sequence, including a peptide, a protein, DNA, RNA, or a gene; an oligomer; a polymer; and any combination thereof. In some embodiments, the one or more active agents can be added to the composition as a co-polymer. In some embodiments, the bioactive agent can be a stem cell, such as a mesenchymal stem cell (MSC). In some embodiments, the bioactive agent can be an antibody, such as a monoclonal antibody (mAb).

The active agent can be attached to a monomer used in the polymerization reactions. In some embodiments, the active agent is a monomer used in the polymerization reactions. In some embodiments, the active agent can be attached to a stabilizer used in the polymerization reactions. In some embodiments, the active agent is a stabilizer used in the polymerization reactions. In some embodiments, the active agent can be attached to a terminator used in the polymerization reactions. In some embodiments, the active agent is a terminator used in the polymerization reactions. In some embodiments, the one or more active agents can be added in a solvent used in the polymerization reactions, and the one or more agents can be soluble or insoluble as a suspension. In some embodiments, the one or more active agents can be linked to the polymer using a linker, for example, to a monomer, to a stabilizer, and/or to a terminator. In some embodiments, the one or more active agents can be attached in a monomer, a stabilizer, a terminator, the reaction solvent, or any combination thereof, used in the polymerization reaction.

The one or more bioactive agents can be added to the compositions through ionic bonding, covalent bonding, hydrogen bonding, mechanical entrapment, or a combination thereof. In some embodiments, the one or more bioactive agents can be linked to the compositions through a linker.

Examples of ionic bonding can include, but are not limited to, ionic bonding of an anionic agent to a cationic site on a polymer or a cationic agent to an anionic site on a polymer. In some embodiments, an anionic agent can be bound to a quaternary amine on a polymer. In other embodiments, an agent with a quaternary amine can be bound to an anionic site on a polymer. Examples of inter-molecular attractions include, but are not limited to, hydrogen bonding such as, for example, the permanent dipole interactions between hydroxyl, amino, carboxyl, and sulfhydryl groups, and combinations thereof. Examples of physical connections can include, but are not limited to, interpenetrating networks and chain entanglement. The agents can also be blended or mixed with the compositions.

In some embodiments, the one or more bioactive agents can be attached to a monomer, a stabilizer, a terminator, or any combination thereof through a labile bond selected from the group consisting of an amide bond, an ester bond, orthoester, an anhydride bond, or a combination thereof. It should be appreciated that the rate of release of one or more active agents can be adjusted through the selection of bonds or bond combinations used to attach the one or more active agents to the polymers taught herein. For example, ester bonds are more labile than amide bonds, orthoester bonds are more labile than ester bonds, and anhydride bonds are more labile than orthoester bonds. In some embodiments, the active agents can be attached through bond that is an ether, an amide, an ester, an anhydride, an orthoester, an all-aromatic carbonate, an acetal, a ketal, a urethane, a urea, a glycoside, a disulfide, a siloxane linkage, or a combination thereof. It is to be appreciated that some of these linkages may not be used in some embodiments. In some embodiments, the active agents have a reactive group that bonds with a reactive group on the polymer to create the desired bond.

Examples of reactive groups on the active agents include, but are not limited to, hydroxyl, acyl, amino, amido, and sulfhydryl groups. In some embodiments, the agents can be released or can separate from the polymer composition.

As used herein, the term “linker”, “linkage” and “linking group” refers to a chemical linking moiety that connects two groups and has a backbone of 50 atoms or less in length. In some embodiments, the linker or linkage may be a covalent bond that connects two groups, or a chemical moiety having a chain of between 1 and 50 atoms in length, between 2 and 20 atoms in length, between 3 and 30 atoms in length, between 4 and 40 atoms in length, between 10 and 50 atoms in length, between 10 and 30 atoms in length, between 12 and 26 atoms in length, between 14 and 30 atoms in length, or any range therein in amounts of 1 atom in length. For example, a linker or a portion of a linker can have a length that is 1, 2, 3, 4, 5, 6, 8, 10, 12, 14, 16, 18, 20, 22, 24, 26, 28, 30, 32, 34, 36, 38, 40, 45, or 50 carbon atoms in length, where the linker may be linear, branched, cyclic, or a single atom. In some embodiments, the linker may be 10 angstroms, 11 angstroms, 12 angstroms, 13 angstroms, 14 angstroms, 15 angstroms, 16 angstroms, 17 angstroms, 18 angstroms, 19 angstroms, 20 angstroms, 22 angstroms, 23 angstroms, 24 angstroms, 25 angstroms, 26 angstroms, 27 angstroms, 28 angstroms, 29 angstroms, 30 angstroms, 32 angstroms, 34 angstroms, 36 angstroms, 38 angstroms, 40 angstroms, 45 angstroms, 50 angstroms, or any amount therein in increments of 1 angstrom, in length. In some embodiments, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo (ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable from either the polymer, the drug, or both the polymer and the drug.

The linking moiety may be conjugated to the polymer and the drug using any convenient functional groups (carboxylic acids, amines, alcohols, carbamates, esters, amide, ethers, thioethers, maleimides, and the like), as taught herein, using known linking chemistries. For example, conjugation chemistry described by G. T. Hermanson (“Bioconjugate Techniques”, Academic Press, Second Edition, 2008) may be readily adapted for use in preparing the subject heterobifunctional compounds and is hereby incorporated herein by reference in it's entirety.

In some embodiments, the activity of an active agent can be an activity that includes, for example, an increase in solubility, flexibility of the polymer, tensile strength of the polymer, or an additive that provides a desirable mechanical property. In some embodiments, the activity of an active agent can be an activity that includes, for example, a prophylactic effect or a therapeutic effect.

In some embodiments, a polymeric agent can be connected as a copolymer and can be any agent taught herein. In some embodiments, the agents include, but are not limited to, poly(alkylene glycols) such as, for example, poly(ethylene glycol) and poly(propylene glycol); phosphorylcholine; poly(N-vinyl pyrrolidone); poly(ethylene oxide); poly(acrylamide methyl propane sulfonic acid); poly(2-hydroxyethyl methacrylate), poly(3-hydroxypropyl methacrylamide), poly(styrene sulfonate); saccharides such as, for example, carboxymethylcellulose; sulfonated polysaccharides such as, for example, sulfonated dextran; sulfated polysaccharides such as, for example, sulfated dextran and dermatan sulfate; and any derivatives, analogs, homologues, congeners, salts, copolymers and combinations thereof. In other embodiments, the agents can include, but are not limited to, glycosaminoglycans such as, for example, hyaluronic acid, heparin, hirudin, dermatan sulfate, chondroitin sulfate; chitin; chitosan; and any derivatives, analogs, homologues, congeners, salts, copolymers and combinations thereof. In other embodiments, the agents can be prohealing such as, for example, poly(ester amides), elastin, silk-elastin, collagen; chondroitin sulfate; peptide sequences such as, for example, atrial natriuretic peptide (ANP), and those comprising Arg-Gly-Asp (RGD); and any derivatives, analogs, homologues, congeners, salts, copolymers and combinations thereof. In other embodiments, the agents can be non-thrombotics such as, for example, thrombomodulin; antimicrobials such as, for example, the organosilanes; and any derivatives, analogs, homologues, congeners, salts, copolymers and combinations thereof.

In some embodiments, the molecular weight of an active agent should be at or below about 40,000 Daltons, or any range therein, to ensure elimination of the agent from a mammal. In one embodiment, the molecular weight of the active agent ranges from about 300 Daltons to about 40,000 Daltons, from about 8,000 Daltons to about 30,000 Daltons, from about 10,000 Daltons to about 20,000 Daltons, or any range therein. It should be appreciated that, if upon release from the polymeric composition, the active agent is rapidly broken down in the body, then the molecular weight of the active agent could be greater than about 40,000 Daltons without compromising patient safety. The molecular weights as taught herein are a number average molecular weight.

The selection of the bond that attaches an active agent allows for control of the relative strength or stability of the bond, and such control allows for a controlled release of agents that are substantially free of attached molecules from the polymeric carrier. It is to be appreciated that the design of a composition for the sustained release of agents can be dependent on a variety of factors such as, for example, the therapeutic, prophylactic, ameliorative or diagnostic needs of a patient. In some embodiments, the agent can comprise an antiproliferative and should have a sustained release ranging from about 1 week to about 10 weeks, from about 2 weeks to about 8 weeks, from about 3 weeks to about 7 weeks, from about 4 weeks to about 6 weeks, and any range therein. In other embodiments, the agent can comprise an anti-inflammatory and should have a sustained release ranging from about 6 hours to about 3 weeks, from about 12 hours to about 2 weeks, from about 18 hours to about 10 days, from about 1 day to about 7 days, from about 2 days to about 6 days, or any range therein. In general, the sustained release should range from about 4 hours to about 12 weeks; alternatively, from about 6 hours to about 10 weeks; or from about 1 day to about 8 weeks.

Effective amounts, for example, may be extrapolated from in vitro or animal model systems. In some embodiments, the agent or combination of agents have a concentration that ranges from about 0.001% to about 75%; from about 0.01% to about 70%; from about 0.1% to about 60%; from about 0.25% to about 60%; from about 0.5% to about 50%; from about 0.75% to about 40%; from about 1.0% to about 30%; from about 2% to about 20%; and any range therein, where the percentage is based on the total weight of the polymer and agent or combination of agents.

### Tissue Healing, Repair, and Regeneration

The active agents can be delivered, for example, in tissue healing, repair, and regeneration. Such uses can include, for example, wound healing of surgical wounds, trauma wounds, pressure sores such as bed sores, and the like, often referred to as primary intention wounds (e.g., surgical incision closed with stitches, wound edges close together), secondary intention wounds (e.g., tooth extraction or severe burn, wound edges too far apart to bring together), and tertiary intention wounds (e.g., dog bite, cleaned and left open to granulate, closed later); tissue grafts, both allograft and autograft, bone, tendon, skin, heart valve blood vessel, cornea, and the like; transplants, e.g., kidney, liver, heart, lung, pancreas, intestine, cornea, bone, skin, tendon, ligament, heart valve, and the like, both allograft and autograft. In some embodiments, a tissue graft can include repair of bone and bone defects, including spinal repair, fusions, increase healing speed and strength, and the like. In some embodiments, a tissue graft can be part of a skin graft, a corneal implant, an organ repair or transplant, dental repair or transplant, or a repair of a ligament or tendon. Ligament repair can include, but is not limited to, anterior cruciate ligament repair or posterior cruciate ligament repair. Tendon repair can include, but is not limited to, repair of a patellar tendon, Achilles tendon, or hamstring. The products and methods taught herein can be used with allografts or autografts, allografts having a higher risk of rejection that could be prevented, at least inhibited, or treated, with compositions delivering the drug delivery agents as taught herein.

Any active agent that can assist in tissue healing, repair, and regeneration can be used in compositions taught herein. The active agent can be used to improve preparation of a surgical wound bed, or improve wound dressings, for example. Biomaterials can be incorporated, fluid balance can be altered, pH can be modified, vascularity can be improved, and the like. Natural products can be used, for example antibiotics are antimicrobials; silver sulfadiazine is an antimicrobial; medicinal honey acts to upregulate proinflammatory cytokines, lower pH, and scavenge reactive oxygen species; polyphenols such as curcumin act as an anti-inflammatory, scavenge reactive oxygen species, and increase collagen synthesis and production; aloe vera acts to scavenge reactive oxygen species, stimulate fibroblast active and proliferation, acts as an anti-inflammatory, and upregulates white blood cells; and birch bark extracts (EPISALVAN) act to increase keratinocyte migration, to name a few. Human-derived factors can also be used, including mesenchymal stem cells (MSCs) that act as immunomodulators, promote cell proliferation and differentiation, promote production of growth factors; macrophages; collagenase (SANTYL) which promotes keratinocyte proliferation and migration; placenta-derived tissue including growth factors (PDGF-BB, TGFα, bFGF and EGF), cytokines (IL-4, IL-6, IL-8 and IL-10) and ECM components; and, autologous leucocyte/platelet/fibrin. A wide variety of drugs can be used, including PDGF (Becaplermin; REGRANEX) which promotes chemotactic recruitment, cellular proliferation, and increases macrophage and fibroblast recruitment; Phenytoin (Diphenylhydantoin; DILATIN) which increases vascularization of granulation tissue and collagen synthesis; Vitamin A/Retinoids (TRETINOIN) which promotes angiogenesis, epithelization, and collagen synthesis; hypochlorous acid which increases keratinocyte migration and vascularization; Pentoxifylline (PTX; TRENTAL) which improves blood flow and oxygenation and increases platelet aggregation; METFORMIN which transitions macrophages from M1 (pro-inflammatory) to M2 (anti-inflammatory); and,4-dihydrophenonthrolin-4-one-3-carboxylic acid (DPCA).

In some embodiments, DPCA can be delivered to assist in tissue regeneration and wound repair. The DPCA can be released from any form of the compositions taught herein. The sodium salt of DPCA, or NaDPCA, can also be used alone or in any combination with DPCA. A powder form of an adhesive composition, for example, can include a powder form of the adhesive compositions taught herein, including one or more active agents mixed in with the powder or chemically attached to a monomer, stabilizer, terminator, and/or linker component of the powder. In some embodiments, the DPCA can be applied to a medical device, for example, such as wound dressing, in some embodiments.

The embodiments provided herein can use either DPCA, NaDPCA, or a combination thereof. These compounds, alone or in combination, can promote tissue regeneration. The DPCA remains in suspension, whereas the sodium salt, NaDPCA, is a hydrophilic salt with more solubility. Rate of delivery can be adjusted, where less solubility results in slower delivery, and so combinations of DPCA/NaDPCA can be used to adjust delivery rates. The total amount of DPCA and/or NaDPCA added to the polymeric compositions taught herein can range from 1% by weight to 20% by weight, from 1% by weight to 15% by weight, from 1% by weight to 10% by weight, from 2% by weight to 20% by weight, from 2% by weight to 15% by weight, from 2% by weight to 10% by weight, from 4% by weight to 20% by weight, from 4% by weight to 15% by weight, from 4% by weight to 10% by weight, from 6% by weight to 20% by weight, from 6% by weight to 15% by weight, from 6% by weight to 10% by weight, from 6% by weight to 8% by weight, or any amount or range therein in increments of 0.1% by weight. In some embodiments, the total amount of DPCA and/or NaDPCA added to the polymeric compositions taught herein can be about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, or any amount or range therein in increments of 0.1% by weight.

In some embodiments, the DPCA can be added to an adhesive composition in an amount based on weight % that can range from about 0.1% to about 20%, from about 0.5% to about 20%, from about 1.0% to about 20%, from about 2.0% to about 20%, from about 3.0% to about 20%, from about 4.0% to about 20%, from about 5.0% to about 20%, from about 6.0% to about 20%, from about 7.0% to about 20%, from about 8.0% to about 20%, from about 9.0% to about 20%, from about 10% to about 20%, from about 12% to about 20%, from about 14% to about 20%, from about 16% to about 20%, from about 18% to about 20%, or any amount or range therein in increments of about 0.1% by weight. In some embodiments, the DPCA can be added to an adhesive composition in an amount that can range from about 0.1% to about 10%, from about 0.5% to about 10%, from about 1.0% to about 10%, from about 2.0% to about 10%, from about 3.0% to about 10%, from about 4.0% to about 10%, from about 5.0% to about 10%, from about 6.0% to about 10%, from about 7.0% to about 10%, from about 8.0% to about 10%, from about 9.0% to about 10%, %, or any amount or range therein in increments of about 0.1% by weight. In some embodiments, the DPCA can be added to an adhesive composition in an amount that can range from about 5.0% to about 7.0%, from about 7.0% to about 9.0%, from about 6.0% to about 8.0%, or any amount or range therein in increments of about 0.1% by weight. In some embodiments, the DPCA is in suspension with the polymer and surrounding fluids to facilitate a desired delivery speed. In some embodiments, the less soluble the active agent, the slower the delivery speed.

### Dental Applications

A drug delivery composition taught herein can be used in dentistry for tissue healing, repair, and regeneration. In some embodiments, a drug delivery composition taught herein can be used in products and methods for periodontal repair, including bone regeneration and regeneration of surrounding tissues. A method of periodontal repair can include, for example, administering a drug delivery composition taught herein into the gum of a subject to regenerate alveolar bone.

### Antibody Treatments

The compositions, systems, and methods provided herein work well to develop antibody treatments for subjects in need. Antibody therapy provides additional bioactive agents that may be useful when administered in combination with the methods taught herein. Any antibody therapy is contemplated in the embodiments herein. In some embodiments, the antibody therapy is an immunotherapy. In some embodiments, the antibody therapy is a cancer therapy. In some embodiments, the antibody therapy is prophylactic, for example, and can be used to inhibit the onset of a condition or disease including, but not limited to, an immunological condition and cancer.

AVASTATIN, for example, is a human monoclonal antibody to VEGF, has provided beneficial results in colorectal cancer, increasing survival time by more than 30% when used in combination with the standard Saltz regime of irinotecan, 5-fluorouracil, and leucovorin. One of skill will appreciate that several monoclonal antibodies would be useful, the following providing further examples of the cancers that can be addressed using the systems and methods taught herein, and any of the bioactive agents taught herein, along with some monoclonal antibody therapies that may also be tested alone or in combination with any of the bioactive agents taught herein:

TABLE 1

| mAb name | Trade name | Cancer treated: |
|---|---|---|
| rituximab | RITUXAN | non-Hodgkin lymphoma |
| trastuzumab | HERCEPTIN | breast cancer |
| gemtuzumab | MYLOTARG | acute myelogenous |
| ozogamicin* | | leukemia (AML) |
| alemtuzumab | CAMPATH | chronic lymphocytic |
| | | leukemia (CLL) |
| ibritumomab tiuxetan* | ZEVALIN | non-Hodgkin lymphoma |
| tositumomab* | BEXXAR | non-Hodgkin lymphoma |
| cetuximab | ERBITUX | colorectal cancer; head |
| | | & neck cancers |
| bevacizumab | AVASTIN | colorectal cancer; non-small |
| | | cell lung cancer; |
| | | breast cancer; glioblastoma; |
| | | kidney cancer |
| panitumumab | VECTIBIX | colorectal cancer |
| ofatumumab | ARZERRA | chronic lymphocytic |
| | | leukemia (CLL) |

*refers to a conjugated monoclonal antibody

Generally speaking, the cancers that can be addressed using the systems and methods taught herein include any known cancer. In addition, or including, the cancers in the list above, the following cancers are of interest: lung cancer, non-small cell lung cancer, adenocarcinoma, squamous cell carcinoma, large cell carcinoma, small cell lung cancer, bronchial cancer, colon cancer, rectal cancer, colorectal cancer, breast cancer, pancreatic cancer, prostate cancer, leukemia, acute myeloid leukemia, lymphoma, Hodgkins lymphoma, non-Hodgkin lymphoma, liver cancer, intrahepatic bile duct cancer, ovarian cancer, and esophageal cancer.

### Other Medical Applications

A variety of medical compositions and uses can incorporate an antiproliferative. Antiproliferatives include, for example, actinomycin D, actinomycin IV, actinomycin 11, actinomycin X1, actinomycin C1, and dactinomycin (COSMEGEN®, Merck & Co., Inc.). Antineoplastics or antimitotics include, for example, paclitaxel (TAXOL®, Bristol-Myers Squibb Co.), docetaxel (TAXOTERE®, Aventis S.A.), methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), and any prodrugs, metabolites, analogs, homologues, congeners, derivatives, salts and combinations thereof.

A variety of medical compositions and uses can incorporate antiplatelets, anticoagulants, antifibrin, and antithrombin. Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein 11b/IlII platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

A variety of medical compositions and uses can incorporate cytostatic or antiproliferative agents. Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, metabolites, analogs, homologues, congeners, derivatives, salts and combinations thereof. Antiallergic agents include, but are not limited to, pemirolast potassium (ALAMAST®, Santen, Inc.), and any prodrugs, metabolites, analogs, homologues, congeners, derivatives, salts and combinations thereof.

Other bioactive agents useful in the medical compositions and uses include, but are not limited to, free radical scavengers; nitric oxide donors; rapamycin; methyl rapamycin; 42-Epi-(tetrazoylyl) rapamycin (ABT-578); everolimus; tacrolimus; 40-O-(2-hydroxy)ethyl-rapamycin; 40-O-(3-hydroxy) propyl-rapamycin; 40-O-[2-(2-hydroxy) ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; and any prodrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (4-hydroxy-TEMPO), 2,2′,3,4,5,5′-hexamethyl-3-imidazolinium-1-yloxy methyl sulfate, free radical; 4-carboxy-2,2′,6,6′-tetramethyl-1-piperinyloxy, free radical (4-carboxy-TEMPO); 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

### Diagnostic Agents

A variety of medical compositions and methods of use can incorporate diagnostic agents. Examples of diagnostic agents include radioopaque materials and include, but are not limited to, materials comprising iodine or iodine-derivatives such as, for example, iohexal and iopamidol, which are detectable by x-rays. Other diagnostic agents such as, for example, radioisotopes, are detectable by tracing radioactive emissions. Other diagnostic agents may include those that are detectable by ultrasound and other imaging procedures such as, for example, fluorescence and positron emission tomography (PET). Examples of agents detectable by ultrasound include, but are not limited to, perflexane. Examples of fluorescence agents include, but are not limited to, indocyanine green. Examples of agents used in diagnostic PET include, but are not limited to, fluorodeoxyglucose, sodium fluoride, methionine, choline, deoxyglucose, butanol, raclopride, spiperone, bromospiperone, carfentanil, and flumazenil.

Other diagnostic agents may include those that are detectable by magnetic resonance imaging (MRI). Examples of agents detectable by MRI are paramagnetic agents, which include, but are not limited to, gadolinium chelated compounds, and magnetic particle imaging (MPI) using one or more MPI agents. MPI can pick up on small abnormalities, so it assists with diagnosing diseases sooner because of the superior sensitivity of the technology. Moreover, a high volume of SPIO tracers is not needed to produce an image, which is a safety improvement, and SPIOs are typically safer for patients anyway. In some embodiments, the SPIO agents are nanoparticles, and these can be referred to as superparamagnetic iron oxide nanoparticles (SPIONs). In some embodiments, the MPI agents can be referred to as “SPIO tracers”.

In some embodiments, MPI agent can be selected from the group consisting of AMI-121, OMP, and AMI-25, and combinations thereof. In some embodiments, the MPI agent can be RESOVIST (by Bayer) which are iron oxide particles coated with carboxydextran. In some embodiments, the MPI agent can be FERUMOXYTOL (aka FERAHEME, by AMI), an iron-based nanoparticle with a carboxymethyl-dextran coating that is FDA approved for treating iron deficiency and used off-label as an MPI agent. In some embodiments, the MPI agent can be ferucarbotran (off-label version of RESOVIST). In some embodiments, the MPI can be VIVOTRAX (also a version of ferucarbotran) and VIVOTRAX+ (magnetically fractionated to select for the population of more magnetic particles to enhance MPI performance in terms of specific signal and resolution) (by GE Healthcare). In some embodiments, the MPI agent is SYNOMAG-D (Manufactured by MMD & Supplied by Gentaur Genprice in USA, UK and Europe), PRECISIONMRX (by Imagion Biosystems, USA and Australia), and PERIMAG, PERIMAG-COOH, PERIMAG-NH2 (by Micromod, modular designed particles, Rostock, Germany).

In some embodiments, SPIONs are essentially tiny particles made of magnetite (Fe₃O₄) or maghemite (Fe₂O₃) with a surface coating like dextran, carboxydextran, or polyethylene glycol, allowing the nanoparticles to be detected within biological fluids like blood due to their strong magnetic response to applied fields.

In some embodiments, MPI agents are used with MRI in tracking the movement of SPIO particles in specific tissues, organs, or areas of the body. In some embodiments, the MPI agents can be used to track cancer. In some embodiments, MPI agents are used with MRI in patients with chronic kidney disease. In some embodiments, MPI agents are used with MRI in angiography. In some embodiments, MPI agents are used with MRI in stem cell tracking. In some embodiments, the MPI agents are used with MRI in real-time fluoroscopy, diagnosis and staging of cancer, in vivo inflammation imaging, and temperature mapping, for example, including measuring a relaxation contrast that reflects a change in temperature.

In some embodiments, a composition for use with MRI, or a method for use with MRI, can include functionalizing SPIO nanoparticles using peptides or antibodies, and the functionalized nanoparticles would adhere to a biological target, such as tumor cells or atherosclerotic plaque.

In some embodiments, a composition for use with MRI, or a method for use with MRI, can be used to track and quantitate inflammation. Inflammation can be targeted by tailoring SPIOs to be uptaken by immune cells using a coating which, in some embodiments, can include standard dextran coatings, for example. In some embodiments, immune cells can be tagged using a method that includes injecting the coated particles and exploiting the reticuloendothelial system (RES) to scavenge the particles, and sites of ongoing inflammation immediately uptake the tracer, such as phagocytic inflammatory cells at sites of tumors and atherosclerotic plaques, and the method can include imaging the particles using MRI. In some embodiments, an MRI scan can be taken several hours later, for example, to reveal other sites of inflammation subsequently targeted by other SPIO-tagged immune cells.

### Drug-Eluting Implants

Implants can carry drugs to treat location-specific issues. The method of treatment can include, for example, selecting one or more drugs, incorporating the drug into a composition taught herein, and delivering the composition to a subject to administer the one or more drugs by delivering the one or more drug to the subject from the polymer.

The implants can have a function other than eluting a drug, or they can have only the function of eluting drug. In some embodiments, implants can carry immunomodulators, antibiotics, antivirals, chemotherapy, growth factors and anti-inflammatory drugs. In some embodiments, such implants can include cardiac implants, vascular implants, gastric implants, dental implants, gynecological implants, orthopedic implants, oncology implants, ophthalmological implants, and dermatological implants. In some embodiments, such implants can include, for example, drug-eluting stents with antiproliferatives; bioelectronics such as pacemakers, pacing leads with steroid coatings, blood glucose monitors, robotic prostheses, vagus nerve stimulators to treat epilepsy and depression, hypoglossal nerve stimulators for sleep apnea; dental implants; condoms coated with spermicides; transdermal drug-delivery patches; drug-eluting wound dressings with antibacterial agents; protein-eluting scaffolds that release growth factors to regenerate tissue; bone cements containing drugs such as antibiotics; drug-coated catheters with heparin, antimicrobials, or antibiotics; and the like. The drug-eluting compositions taught herein can be applied to the implants which can be made from any of a variety of materials, for example, metal alloys (titanium, titanium alloys, stainless steel 316L, cobalt chrome, tantalum, polyethylene), ceramics (zirconium ceramics, alumina ceramics), polymers, cellulosic materials, and combinations thereof.

The cardiovascular implants that can use the drug-eluting compositions taught herein to deliver one or more active agents include stents, in some embodiments. In some embodiments, the cardiovascular implant can include a filter. Drugs that can be added to the compositions taught herein and eluted by a stent include, for example, antiproliferative agents such as SIROLIMUS, EVEROLIMUS, ZOTAROLIMUS, ZOTAROLIMUS, PACLITAXEL, and BIOLIMUS, which act as immunomodulators and cell growth inhibitors. Drugs eluted from a stent can include any antirestenosis agent, in some embodiments. In some embodiments, any drug taught herein can be used.

In some embodiments, drugs used for the treatment of pulmonary arterial hypertension (PAH) are provided and can include, but are not limited to epoprostenol, riociguat, bosentan, macitentan, ambrisentan, treprostinil, sildenafil, tadalafil, selexipag, and iloprost, and combinations thereof. Each of these drugs can be administered alone, or in combination, in some embodiments.

The gynecological implants that can use the drug-eluting compositions taught herein to deliver one or more active agents include subcutaneous implants or intra-uteral implants. In some embodiments, the gynecological implants can elute a contraceptive, an anti-inflammatory, an antibiotic, or a combination thereof. In some embodiments, the gynecological implant is a non-invasive drug-eluting ring implant that is inserted into the vagina to elute one or more desired drug. In some embodiments, the one or more active agents eluted from an adhesive composition used with a gynecological implant can be selected from the group consisting of progestin, aprogestin, or a combination thereof. In some embodiments, the one or active agents can be a gonadotropin-releasing hormone (GnRH) agonist, or a combination of such agonists.

The oncological implants that can use the drug-eluting compositions taught herein to deliver one or more active agents include implants that provide adjuvant therapy for chemotherapy, radiation and surgery. In some embodiments, the oncological implant elutes one or more active agents to suppress tumor formation locally, overcoming side effects associated with systemic chemotherapy treatment and reduce the need for surgical resection of cancerous tumors. In some embodiments, any cancer treatment agent taught herein can be used with the adhesive compositions in an oncological implant, including, for example, the antiproliferatives, apoptotics, immunomodulators. In some embodiments, the one or active agents can be a gonadotropin-releasing hormone (GnRH) agonist, or a combination of such agonists. In some embodiments, the one or more active agents can be selected from the group consisting of carboplatin, cisplatin, fludarabine, vincristine, alkylating antineoplastic agent, bendamustine, cladribine, epirubicin, fluorouracil, cyclophosphamide, hydroxyurea, mercaptopurine, antimetabolite, busulfan, dacarbazine, etoposide, methotrexate, daunorubicin, paclitaxel, adriamycin, azacitidine, capecitabine, docetaxel, idarubicin, and combinations thereof.

The ophthalmological implants that can use the drug-eluting compositions taught herein to deliver one or more active agents include contact lenses and intravitreal implants, for example. In some embodiments, the opthalmological implants can deliver drugs as targeted doses, extended doses, and combinations thereof, to the retina without the need for injection. In some embodiments, the one or more active agents eluted from an ophthalmological implant can be selected from the group consisting of fluocinolone acetonide, used to treat non-infectious uveitis; ganciclovir; dexamethasone; bimatoprost; and combinations thereof. In some embodiments, the one or more active agents incorporated in an ophthalmological implant can be selected from the group consisting of cidofovir, clindamycin, triamcinolone, bevacizumab, ranibizumab, aflibercept, and combinations thereof.

The dermatological implants that can use the drug-eluting compositions taught herein to deliver one or more active agents include drug eluting sutures. In some embodiments, the dermatological implants can produce a prolonged local release of anesthetic, heal wounds, or a combination thereof. In some embodiments, the adhesive compositions themselves can elute drugs and be used to supplement or replace sutures.

One or more active drugs can be administered using a cannula to insert a device under the skin of a subject for a localized drug dosing, and the device can be resorbable or removable. In some embodiments, device can be administered to treat opioid addiction by administering methadone or buprenorphine to reduce withdrawal symptoms and control cravings. In some embodiments, device can be administered to treat Parkinson's disease. In some embodiments, device can be administered to treat thyroid insufficiency. In some embodiments, device can be administered to elute birth control drugs. In some embodiments, device can be administered to elute an antiviral agent. In some embodiments, device can be administered to elute an antimalarial agent, such as a chemoprophylactic. In some embodiments, device can be administered to elute a veterinary medication in an animal.

In some embodiments, the one or more active agents can be selected to inhibit the onset of, reduce the amount of, or treat solid cancers or liquid cancers. In some embodiments, the one or more active agents can be selected to inhibit the onset of, reduce the amount of, or treat angiogenesis associated with cancer growth. In some embodiments, the one or more active agents can be selected to promote the onset of, increase the amount of, angiogenesis associated with tissue regeneration in wound healing.

The orthopedic implants that can use the drug-eluting compositions taught herein to deliver one or more active agents including screws, plates (buttress, neutralization, bridging, tension, compression), and prostheses (replace missing bones or joints, like knees and hips). In some embodiments, the orthopedic implants elute antibiotics, antimicrobials, anti-infectives, anti-inflammatoires, or a combination thereof.

In some embodiments, the one or more active agents can be selected to promote the onset of, increase the amount of, bone regeneration in bone healing. In some embodiments, the active agent is selected to aid in the regeneration of bone at implantation sites while reducing microbial growth. In some embodiments, the one or more drugs can be selected from the group consisting of bisphosphonates, teriparatide, statins, angiotensin-converting enzyme inhibitors, beta-blockers, nitrites, and thiazide diuretics, or a combination thereof, as promoters of osseointegration. In some embodiments, the compositions taught herein can be used to deliver mesenchymal stem cells (MSCs), specifically those derived from bone marrow (BM-MSCs), adipose-derived stem cells (ADSCs), umbilical cord blood stem cells, and skeletal stem cells (SSCs), or a combination thereof, all of which have the potential to differentiate into bone-forming cells.

In some embodiments, the one or more drugs can be selected from the group consisting of loop diuretics, non-steroidal anti-inflammatory drugs, corticosteroids, cyclosporine A, cisplatin, methotrexate, antibiotics, proton pump inhibitors (PPIs), antiepileptics, selective serotonin reuptake inhibitors (SSRIs), and anticoagulants, or a combination thereof, as inhibitors of the osseointegration process.

In some embodiments, the one or more active agents inhibit the onset of, or reduce the onset of, or treat, immunorejection of tissue or implants. In some embodiments, the one or more active agents are indicated for immunosuppression rejection, cellular rejection treatment; immunosuppression maintenance; rejection treatment, immunosuppression maintenance; malignancies, rejection treatment, immunosuppression maintenance; immunosuppression induction, and combinations thereof. In some embodiments, the one or more immunorejection agents are immunosuppressants selected from the group consisting of corticosteroids, calcineurin inhibiter, anti-metabolite, a target of a rapamycin inhibiter, a T-cell depleting monoclonal antibody, and combinations thereof. In some embodiments, the one or more immunorejection agents are immunosuppressants selected from the group consisting of prednisone, cyclosporine, tacrolimus, azathioprine, mycophenolate mofetil, sirolimus, everolimus, alemtuzumab, and combinations thereof.

In some embodiments, the one or more active agents can be selected to inhibit the onset of, reduce the amount of, or treat restenosis from vascular implants. In some embodiments, the active agents can be selected from the group consisting of drugs that inhibit coagulation, such as anticoagulants, antithrombotic agents, antiproliferatives, anti-inflammatory, and antiplatelet agents. In some embodiments, the one or more active agents can be selected from the group consisting of clopidogrel and heparin, and combinations thereof. In some embodiments, the one or more active agents can be selected from anti-inflammatory agents such as dexamethasone and tranilast, and combinations thereof. In some embodiments, the one or more active agents can be immunosuppressants selected from the group consisting of prednisone, cyclosporine, tacrolimus, azathioprine, mycophenolate mofetil, sirolimus, everolimus, alemtuzumab, and combinations thereof. In some embodiments, the one or more active agents are selected from the group consisting of paclitaxel, sirolimus, and combination thereof.

### EXAMPLES

### Examples 1-21 are Polymers of Poly(Lipoic Acid) and Methods of Making the Polymers And Products of the Polymers

Poly(α-lipoic acid) has attracted significant attention due to its excellent mechanical properties and adhesion strength, as well as a cost-effective production and closed-loop chemical recycling. It's found in nature, and is safe, but depolymerizes quickly. Despite multiple attempts, the art has not been able to create a stable form of poly(α-lipoic acid) having these desired features. The following examples show that the instant technology provides such poly(α-lipoic acid) compositions, methods of making, and products. These examples teach that a poly(α-lipoic acid) can be provided as (i) a precursor solution of monomer used to form the poly(α-lipoic acid); (ii) a solution of poly(α-lipoic acid); (iii) a freeze-dried form of the poly(α-lipoic acid) that can be reconstituted as a spray in solution or formed into a solid adhesive product; (iv) a combination of a poly(α-lipoic acid) and asparagusic acid monomer in solution; or any combination thereof.

### Example 1. An Illustration of the Rapid Depolymerization of α-Lipoic Acid (L1) Monomer in Aqueous Solution that Occurs in Nature

This example is creating a baseline polydisulfide system that shows the depolymerization problem in aqueous solution, wherein the polydisulfide system includes α-lipoic acid (L1) added to an aqueous phosphate buffer to produce a rapid polymerization and depolymerization of polydisulfide polymer.

### Experimental:

An ethanolic solution of pure α-lipoic acid L1 was prepared at 400 mg/ml and added to an aqueous phosphate buffer. A metastable sticky polymer was obtained, but within a few minutes (<5 min), the sticky polymer converted to a solid monomeric precipitate with no apparent adhesive property. These observations suggest that rapid polymerization of L1 upon contact with water was followed by rapid depolymerization of the polymer product, compromising its efficacy as a practical adhesive or sealant.

### Example 2. Stabilizing the Polymer to Depolymerization by Adding a Derivatized α-Lipoic Acid Monomer as a Stabilizing Molecule in a Slow Polymerization System

This example is solving the depolymerization problem by derivatizing the α-lipoic acid (L1) monomer with N-hydroxysuccinimide (NHS) ester to create a stabilizer molecule (L2), the NHS-derivative of the the α-lipoic acid (L1) monomer. The stabilizer is added to an absolute ethanol solution of the α-lipoic acid (L1) that includes a catalytic amount of DMSO for a slow polymerization system that shows stabilization of the polymer by the derivatized α-lipoic acid monomer.

### Experimental:

An ethanolic solution of the mixture of α-lipoic acid L1 and L2 was prepared with a mol ratio L1:L2 of 93:7, and a catalytic amount of DMSO was added to ensure the solubility of the NHS esters. The ethanolic adhesive precursor solution prepared in this manner with a total concentration (L1+L2) of 400 mg/mL was observed to form a gel within 1 hour. In contrast, a pure L1 solution at a similar concentration did not form a gel even at extended periods of time. The total (L1+L2) concentration investigated ranged from a low of 200 mg/mL to a high of 600 mg/mL; we observed a decrease in gelation time with an increase in monomer concentration. The formation of a gel indicate that polymerization has occurred and that the polymer produced is stable, clearly establishing the efficacy of NHS esters to stabilize the polymer chain end and prevent rapid depolymerization of the polymer product.

### Example 3. Testing the Ability of the Derivatized α-Lipoic Acid Monomer to Stabilize the Polydisulfide Polymer in a Fast Polymerization System

Water is added to the system of Example 2 to create a fast polymerization system, and a stable polymer is again obtained.

### Experimental:

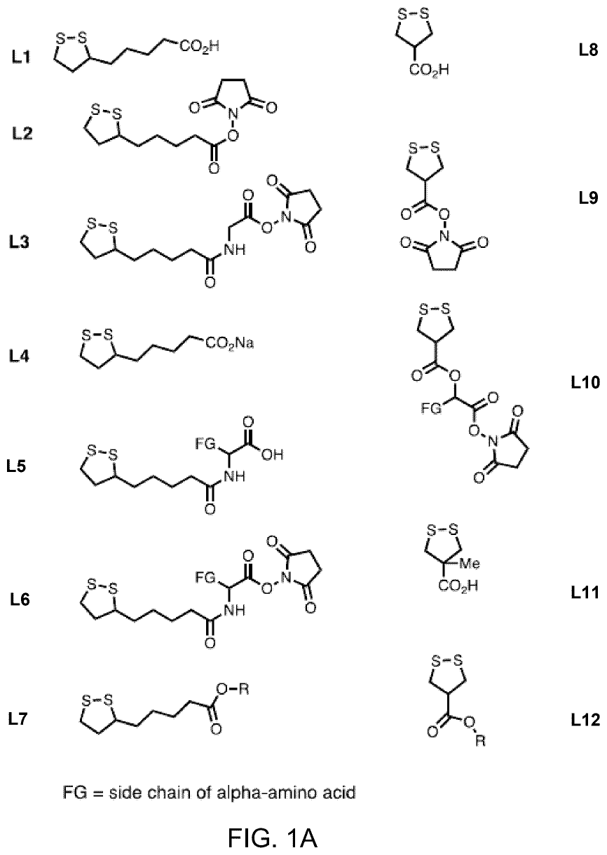

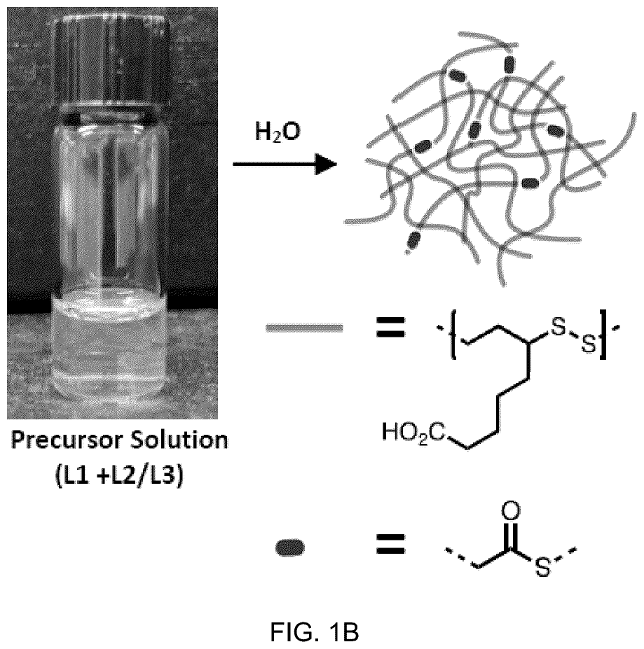

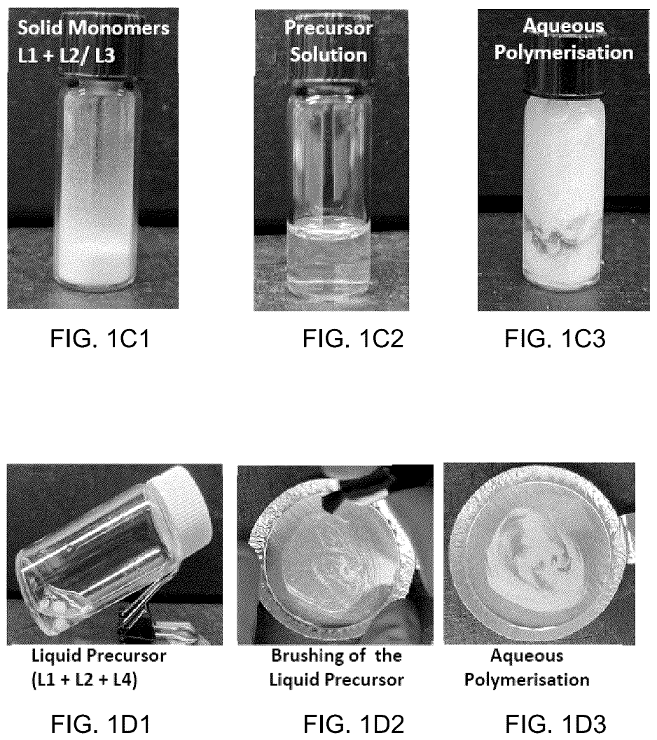

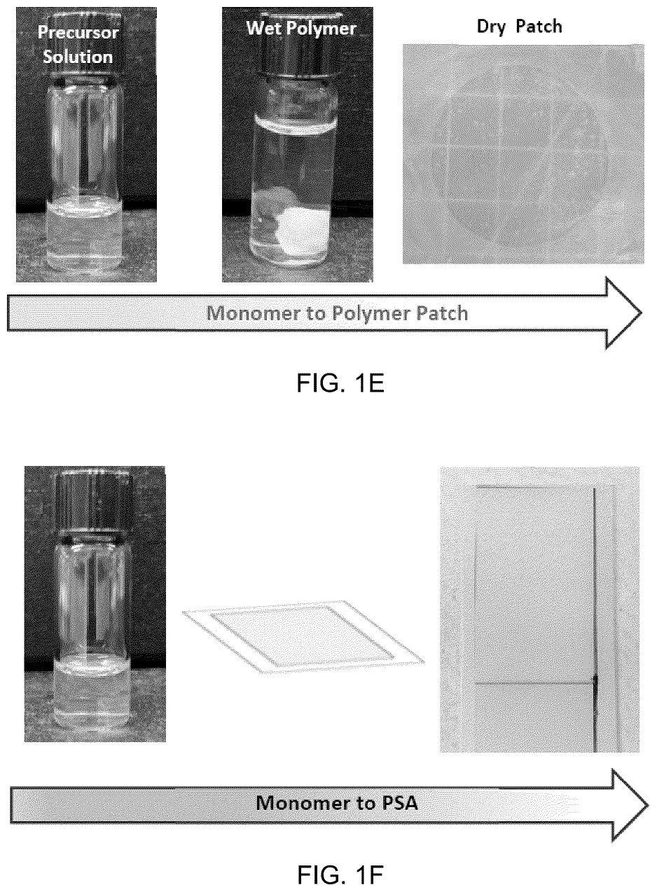

At any time prior to or after gelation of the precursor solution, exposure to water triggers dithiolane polymerization which can be exploited for the purposes of isolating a polymer adhesive or for adhering objects together. An ethanolic liquid mixture of α-lipoic acid L1 and L2 exposed to water led to the immediate formation of a stable sticky solid (FIG. 1). The differential scanning calorimetry analysis of the obtained wet polymer showed an obvious glass transition (Tg) of around −29° C., indicating the formation of a polymer. We believe α-lipoic acid undergoes self-catalyzed polymerization followed by stabilization with NHS esters. Nonetheless, the aqueous polymerization appears to be less sensitive and extremely fast irrespective of the solution concentrations. We believe that due to the amphiphilic nature of the α-lipoic acid, it undergoes micelle-like self-reorganization in contact with water, which locally concentrates the cyclic backbone to accelerate the polymerization rate. Furthermore, detailed ¹H NMR experiments of diluted ethanolic solution of the mixtures show excellent stability of the NHS esters even at an extended period. Therefore, this opens the possibility of an on-demand aqueous polymerization from a precursor ethanolic solution, which we considered to be advantageous for translation in medical and other underwater applications.

### Example 4. Designing and Testing a Second Derivatized α-Lipoic Acid Monomer (L3) Having a Higher Electrophilicity than the NHS-Derivatized α-Lipoic Acid Monomer (L2) and Testing L3 for It's Strength as a Stabilizer

This example adds the stabilizer molecule (L3) to the slow polymerization system that includes α-lipoic acid (L1) in absolute ethanol, with and without the catalytic amount of DMSO, to create a stable polydisulfide polymer. In addition, a faster polymerization was obtained using L3 instead of L2, and it's believed this is due to the higher electrophilicity of L3. Polymerization time was also decreased in the presence of DMSO.

### Experimental:

An ethanolic solution of a mixture of α-lipoic acid L1 and L3 was prepared with a mol ratio L1:L3 of 93:7, and a catalytic amount of DMSO was added to ensure the solubility of the NHS esters. The ethanolic adhesive precursor solution prepared in this manner had a total concentration (L1+L3) of 400 mg/ml and was observed to form a gel within 30 minutes. The total (L1+L3) concentration investigated ranged from a low of 200 mg/mL to a high of 1000 mg/mL; we observed a decrease in gelation time with an increase in monomer concentration. The faster gelation time for L1+L3 compared to L1+L2 was attributed to the higher electrophilicity of L3. Moreover, the presence of a catalytic amount of DMSO also appeared to enhance the rate of solution polymerization, as a DMSO-free ethanolic solution of L1 and L3 showed much slower gelation kinetics than a DMSO-containing solution. The positive influence of polar aprotic solvents in substitution reactions is well known in the literature and we believe the presence of a catalytic amount of DMSO enhances the chain end coupling with the NHS ester, leading to faster polymerization.

### Example 5. Stepwise Polymerization and stFIGabilization of Poly(α-Lipoic Acid)

The examples above react the α-lipoic acid (L1) with the Gly-OSu derivative of α-lipoic acid stabilizer (L3), such that polymerization of the α-lipoic acid (L1) occurs concurrent to the stabilization of polymer by reacting the polymer with the α-lipoic acid stabilizer (L3). This example allows for polymerization to occur in a first step, and the stabilization to occur as a second step.

### Experimental:

An ethanolic solution of pure α-lipoic acid (L1) was prepared at 400 mg/mL, and 5 mol % HCl (4M in dioxane) was added. The obtained mixture was stirred for 15 min, and a gradual increase in viscosity was observed, indicating polymerization of lipoic acid. Subsequently, the Gly-OSu derivative of lipoic acid (L3) was dissolved in a small amount of DMSO and added to the reaction mixture (mol ratio of L1:L3 of 93:7) to stabilize the polymer. The obtained solution mixture was further polymerized by addition of excess 1× PBS. The obtained precipitate was freeze-dried to obtain a pale yellow dry polymer. It should be appreciated that stepwise synthesis of a stabilized polymer may allow for control over the size of the polymer created prior to addition of the stabilizer which is reasonable to expect is a variable that may also affect polymer size when present during the polymerization of the monomer.

### Example 6. Testing Other Electrophilic Molecules as Stabilizers

This example tests hexamethylene diisocyanate, benzoquinone, maleic anhydride, and acetyl-glycine N-hydroxysuccinimide ester as stabilizers. Polymer was formed in an hour in the slow polymerization system that includes α-lipoic acid (L1) in absolute ethanol with and without catalytic amount of DMSO. In each case, a stable polymer was obtained, and water was added to the reaction with maleic anhydride to show that a stable polymer was still obtained in the fast polymerization system.

### Experimental:

An ethanolic solution of a mixture of α-lipoic acid L1 and hexamethylene diisocyanate was prepared with a mol ratio L1: hexamethylene diisocyanate of 90:10, and a catalytic amount of DMSO was added. The ethanolic adhesive precursor solution prepared in this manner had a total concentration (L1+hexamethylene diisocyanate) of 400 mg/ml and was observed to form a gel within 15 minutes.

An ethanolic solution of a mixture of α-lipoic acid L1 and 1,4 benzoquinone was prepared with a mol ratio L1: benzoquinone of 90:10, and a catalytic amount of DMSO was added. The ethanolic adhesive precursor solution prepared in this manner had a total concentration (L1+benzoquinone) of 400 mg/ml and was observed to form a gel within 30 minutes.

An ethanolic solution of a mixture of α-lipoic acid L1 and acetyl-glycine N-hydroxysuccinimide ester was prepared with a mol ratio L1: acetyl-glycine N-hydroxysuccinimide ester of 93:7, and a catalytic amount of DMSO was added. The ethanolic adhesive precursor solution prepared in this manner had a total concentration (L1+acetyl-glycine N-hydroxysuccinimide ester) of 400 mg/ml and was observed to form a gel within 30 min.

An ethanolic solution of a mixture of α-lipoic acid L1 and maleic anhydride was prepared with a mol ratio L1: maleic anhydride of 93:7, and a catalytic amount of DMSO was added. The ethanolic adhesive precursor solution prepared in this manner had a total concentration (L1+maleic anhydride) of 400 mg/ml. Thereupon, the precursor solution was exposed to water to trigger dithiolane polymerization to obtain a stable sticky solid (FIG. 1).

### Example 7. Creating a Stable Liquid Precursor by Suppressing Initiation of Polymerization

This example offers a DMSO solution of monomers that suppresses polymerization of the monomers until water is added. The α-lipoic acid (L1) monomer is added to methanol with a catalytic amount of DMSO, the NHS-derivatized α-lipoic acid monomer (L2), and a conjugate base of the α-lipoic acid (L1) monomer, sodium lipoate (L4), was added to compete with the tendency of the disulfide groups to self-polymerize. The NHS-derivative of the α-lipoic acid (L1) monomer was added to stabilize the polymer. Methanol was evaporated at low vacuum at temperature<30° C. to obtain a liquid precursor solution. The solution was successful, stable for a month at 5° C., and the polymerization is ultrafast with the addition of water.

### Experimental:

The self-catalysis of α-lipoic acid takes place by the activation of disulfide with a carboxylic acid. Thus, we hypothesized that introduction of a functional group with higher electron density than disulfide will act as a potential competitor of disulfide, thereby suppressing initiation. To test our hypothesis, a mixture of L1, sodium lipoate (L4), and L2 (mol ratio: 70:23:7) were dissolved in methanol with a catalytic amount of DMSO to obtain a homogeneous solution. Interestingly, a viscous honey-like yellow liquid was obtained after the complete evaporation of the solvent (FIG. 1). ¹H NMR analysis of the obtained liquid confirmed the monomeric state of the mixture. The obtained liquid shows excellent stability, as no apparent change in viscosity was observed over a month when stored at 5° C. However, the liquid undergoes ultrafast polymerization when brought into contact with water (FIG. 1) to yield an adhesive polymer. We surmise that due to its higher hydration energy, L4 becomes solvated as it comes in contact with water, leading to disulfide activation by the carboxylic acids. This approach provides an adhesive precursor suitable for spray, brush or injection mode of application.

### Example 8. Creating a Solid, Adhesive Polymer Patch

A solution of the α-lipoic acid (L1) monomer in absolute ethanol was added to a solution of the NHS-derivatized α-lipoic acid (L2) in DMSO. Excess phosphate-buffered saline (PBS) was added to polymerize the monomer, and a stable polymer was created, freeze-dried, and compression molded between polydimethylsiloxane (PDMS) coated paper to create a thin film solid adhesive patch having desirable mechanical properties suitable for many applications, including significant stretch without rupture, stiffness suitable for use on biological tissue, high hysteresis, excellent fatigue resistance, excellent stress relaxation, resistance to deformity, and self-healing, for example.

### Experimental:

α-Lipoic acid (900 mg, 4.4 mmol) was dissolved in 2.3 mL absolute ethanol. Separately, L2 (100 mg, 0.3 mmol) was dissolved in 200 μL DMSO and added to the lipoic acid solution. The resulting solution was rested for 1 hour before adding excess 1× PBS (pH 7.4, temperature 37° C.) under vigorous stirring. The sticky resulting polymer was washed with fresh PBS, followed by miliQ water. The obtained pale-yellow polymer was freeze-dried to obtain dry polymer and then compression molded at 75° C. for 15 min between PDMS backing paper to obtain a homogeneous thin film (thickness 250±50 μm) (FIG. 1). The polymer films were rested overnight before further use.

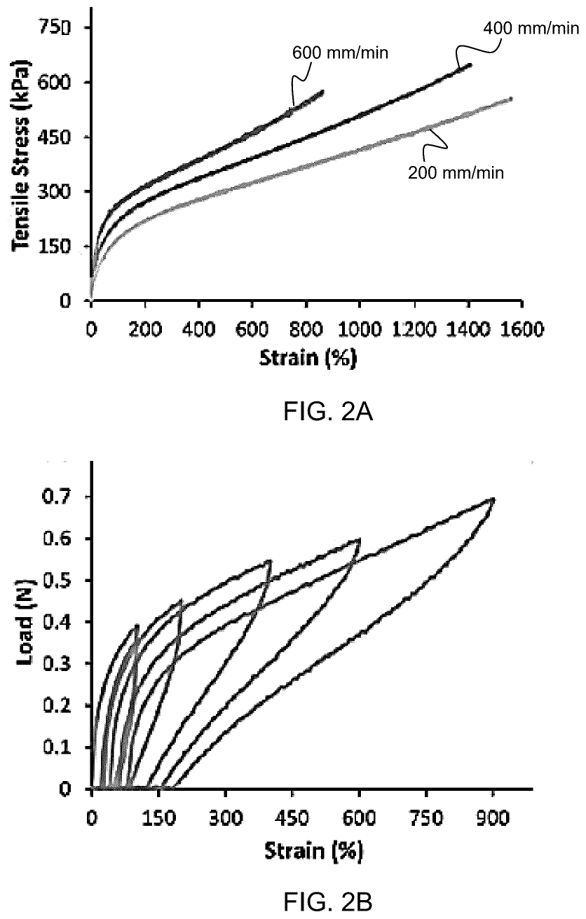

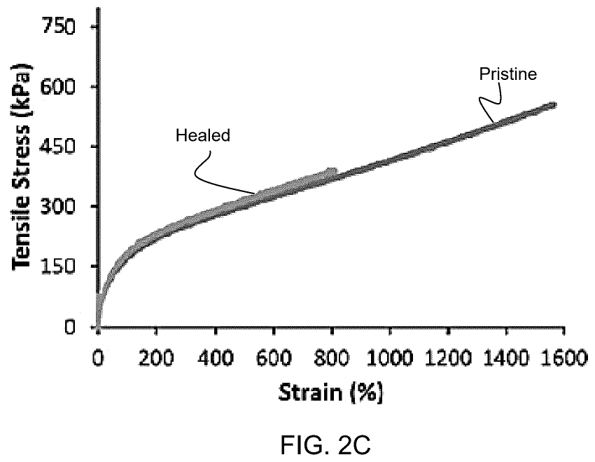

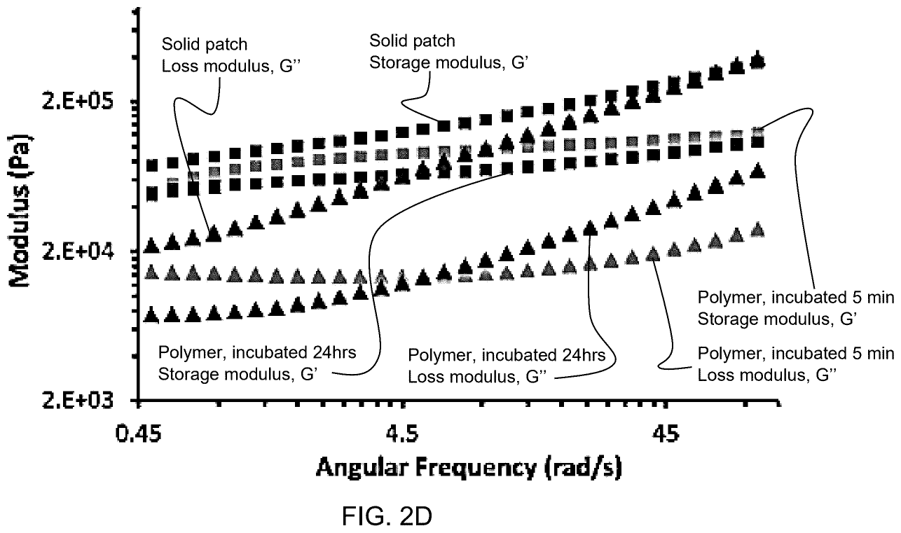

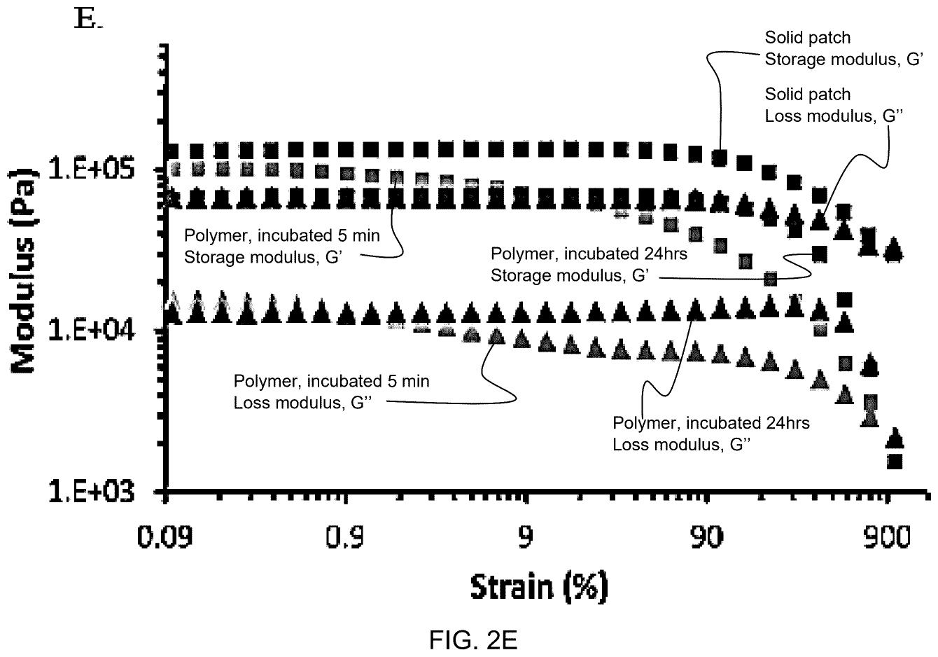

Stress-strain measurements of the resulting polymer patch show the polymer could be stretched over 10 times of its initial length (1000% strain deformation) without rupture (See FIG. 2A). The young's modulus (481±60 kPa) was calculated from the lower region of the stress-strain curve obtained at 200 mm/min, demonstrating the stiffness of the adhesive as being within the range of many biological tissues. The stress-strain measurements with increasing strain rates revealed higher stiffness of the polymer, indicating the viscoelastic nature of the polymer (See FIG. 2A). Furthermore, the cyclic stress-strain measurements also display a high hysteresis, a characteristic feature of viscoelastic polymer. The polymer shows excellent recovery at the lower deformation region, whereas another cyclic stress-strain measurement with subsequent incremental strain without intermediate resting indicates excellent fatigue resistance of the obtained polymer (See FIG. 2B).

A stress relaxation experiment with an instantaneous strain of 100% shows the polymer relaxes from 576±20 kPa stress to 118±5 kPa within 18 sec, thereby highlighting the excellent stress relaxation property of the polymer. Furthermore, the creep test shows that the polymer can bear an 80 kPa load for an extended time without undergoing deformations. Next, the self-healing property of the polymer was investigated. The poly(α-lipoic acid) copolymers are known to undergo fast self-healing due to the presence of dynamic covalent bonds. Therefore, to test the healing efficiency, a polymer film was cut into two pieces and then carefully brought into contact for 2 min at 37° C. and equilibrated for 5 min at room temperature before measurements. Interestingly, over 60% recovery efficiency was achieved within such a short interval, indicating high chain mobility within the polymer network (See FIG. 2C). Afterward, the rheological properties of the polymer were also analyzed for both wet and thermally processed polymer (See FIG. 2D, 2E). An amplitude sweep analysis of the dry polymer shows an extended viscoelastic region, whereas the wet polymer immediately after polymerization shows a smaller linear viscoelastic region (See FIG. 2E). However, an extended viscoelastic region was observed when the polymer was incubated for 24 h (See FIG. 2E). We believe, initially, the polymer network has a lower crosslinking density, and it increases over time and provides higher stability of the network. A frequency sweep, on the other hand, shows only a slight reduction in modulus in the wet state compared to the dry state and was attributed to the lower water uptake by the polymer due to the higher hydrophobicity of the poly(α-lipoic acid) (See FIG. 2D).

### Example 9. Testing Adhesion Between Biological Tissues as a Replacement for Medical Grade Cyanoacrylate Superglues

The α-lipoic acid (L1) monomer was combined with the NHS-derivative of the α-lipoic acid (L2) in absolute ethanol with a catalytic amount of DMSO. The mixture was applied to two bovine heart tissue surfaces as an atomized spray to evaporate the ethanol and increase the concentration of the mixture. Lap joints were created immediately between the tissue and incubated in PBS for further curing. The adhesive outperformed medical grade cyanoacrylate superglue at higher incubation times. Changing the stabilizer molecule from the NHS-derivative of the α-lipoic acid (L2) to the Ac-Gly-OSu-derivative of the α-lipoic acid (L3) didn't significantly affect the results. Likewise, doubling the concentration of the NHS-derivative of the α-lipoic acid (L2) did not significantly affect the results. Ultrafast polymerization upon contact with a wet tissue surface, combined with high mechanical strength and high stretchability makes the instant technology an attractive alternative to medical-grade cyanoacrylate superglue, for example.

### Experimental:

An atomized spray was initially chosen, as it allows for quick evaporation of ethanol to increase the concentration of the mixture rapidly and eliminate any undesired flow of the adhesive solution on the tissue surfaces. A solution of L1 and L2 (7 mol %) was prepared and sprayed on two pieces of tissue surfaces (40 μL in 1 cm²) and the two pieces were immediately brought into contact. After forming the lap joints, they were incubated for 2 minutes in PBS at 37° C. for further curing of the polymer network. The lap shear strength after 2 minutes on wet bovine pericardium tissue was >100 kPa, which was only slightly lower than medical-grade superglue (See FIG. 3A). Interestingly, when the lap joints were incubated for 24 hours in PBS at 37° C., the shear strength increased almost two-fold to >200 kPa, thus outperforming medical-grade superglue (See FIG. 3A). The failure mode analyses of the lap joints was performed by applying N-bromo succinimide to the failed tissue surfaces to visually indicate by charge transfer complexation of disulfide functionality and therefore the presence of adhesive polymer. This analysis revealed cohesive failure both at 2 min and 24 h incubation, suggesting strong adhesion at the tissue-adhesive interfaces. The enhanced lap shear strength at longer incubation time is consistent with the enhanced cohesive strength of the polymer network as observed in the rheology analysis of the wet polymer (see FIG. 2E).

Lap shear tissue adhesion was also performed with L3 on both wet bovine pericardium and pig skin. Like L2, a high shear strength was also observed within 2 min incubation in PBS, and an almost two-fold increase was observed after 24 hours of incubation (See FIG. 3B). Similarly, the peel test analyses on the pig skin also showed excellent peel strength within 5 min incubation in PBS and increased significantly after 24h.

To study the effect of stabilizer concentration on tissue adhesion strength, another lap shear test was performed with a higher L2 concentration (14 mol %). No significant difference in the shear strength was observed.

### Example 10. Testing Adhesion, Namely Heterobonding Between Biological Tissues and Various Substrates that Include Hard, Soft, Organic, and Inorganic Substrates

These substrates include stainless steel (SS), titanium dioxide (TiO2), glass (SiO2), nylon, high density polyethylene (HDPE), polydimethylsiloxane (PDMS), and polytetrafluoroethylene (PTFE). The α-lipoic acid (L1) monomer was combined with the NHS-derivative of the α-lipoic acid (L2) in absolute ethanol with a catalytic amount of DMSO. The mixture was applied to form joints between heart tissue and the substrates. There was excellent bonding in all joints, including the HDPE and PTFE substrates, meaning that the adhesives can replace the medical grade cyanoacrylates, as well as offer the surprising feature that they're expected to work well in vascular grafting procedures.

### Experimental:

A lap joint was fabricated using the ethanolic adhesive precursor of L1 and L2, where wet bovine pericardium tissue was taken as one component of the lap joint, whereas the other component was selected from a range of hard and soft, organic and inorganic substrates. Interestingly, excellent adhesion strengths were observed with both metallic and polymeric substrates (See FIG. 3E). This result suggests the remarkable ability of our adhesive to mediate bonding on various medical devices to tissue. It is also worth mentioning that the high adhesion strength of tissue with polymeric substrates like PE and PTFE opens the possibility of using the newly developed adhesives in vascular grafting.

### Example 11. Comparing the Strength of 2 Different Adhesives on Pig Skin and Cow Heart Tissue

This example compares the strength of 2 different adhesives on pig skin and cow heart tissue, namely comparing (i) a brushing of the stable liquid precursor having L1, L2, and L4 in DMSO solution (“the brush adhesive”) to (ii) a spraying of the adhesive having the L1 and L2 in absolute ethanol solution (“the spray adhesive”). At 2 minutes incubation in PBS the adhesion strengths between the 2 different adhesives are comparable. At 24 hours incubation in PBS, the spray adhesive was significantly stronger than the brush adhesive, assumed due to the lower carboxylic acid concentration in the stable liquid precursor used in the brush adhesive due to the addition of the conjugate base of the lipoic acid (L4). Even with the lower adhesion strength, however, the brush adhesive remains comparable to the medical grade cyanoacrylate adhesive.

### Experimental:

The shear strength of the ethanol-free liquid precursor was also tested on wet bovine pericardium and pig skin. The lap joints were prepared by brushing (5 mg in 1 cm²) the liquid precursor of L1, L2 and L4 mixture on the wet tissue surface, followed by incubation in PBS for 2 min and 24 hours at 37° C. The lap shear measurements after 2 min incubation in PBS shows a high adhesion strength with pig skin and was comparable to spray mode. A slightly lower adhesion strength was observed in the case of the bovine pericardium (See FIG. 3D). A much higher difference in shear strength was observed among spray and brush modes after 24 hours of incubation in both pig skin and bovine pericardium. We believe the lower carboxylic acid concentration in liquid precursor reduced the number of noncovalent interactions with tissue surface and within the polymer network, therefore resulting in lower adhesion strength than the spray mode of application. Even though the brush mode shows lower adhesion strength than the spray, it is still comparable to medical-grade cyanoacrylate.

### Example 12. Testing the Adhesion Strength of the Solid Polymer Patch Made from a Solution of The α-Lipoic Acid (L1) Monomer in Absolute Ethanol Added to a Solution of the NHS-Derivatized α-Lipoic Acid (L2) in DMSO

The solution of the α-lipoic acid (L1) monomer in absolute ethanol added to a solution of the NHS-derivatized α-lipoic acid (L2) in DMSO was added to PBS, dried, and formed as a patch as described above. After only 2 minutes curing time, the solid patch was not as strong as the liquid spray and liquid brush adhesives of Example 11, but the shear strength of the solid patch approached that of the liquid spray and liquid brush adhesives after 24 hours. It is suspected that the dynamic covalent bonds in the solid patch are hindered by the high hydrophobicity of the patch and low mobility of the polymers, but the molecules are mobile and reorganize over the 24 hours to interact with the tissue surface. The lower strength observed in the first 2 minutes was resolved by spraying or brushing the solid polymer patch with a liquid form of the adhesive that was made by combining the α-lipoic acid (L1) monomer in absolute ethanol with a solution of the Ac-Gly-OSu-derivatized α-lipoic acid (L3) in DMSO.

### Experimental:

Contrary to both liquid modes, the solid polymer patch shows significantly lower adhesion strength after 2 min incubation in PBS (see FIG. 3C). However, the shear strength increased significantly after 24 hours of incubation in PBS. Without intending to be bound by any theory or mechanism of action, we suspect that the high hydrophobicity and lower chain mobility in the solid state hindered the interaction of the polymer surface with wet tissue. Likewise, we suspect that the presence of highly dynamic covalent bonds in the network allows the reorganization of the network to obtain favorable interaction with the tissue surface; hence, the shear strength increases over time. To address the low initial patch adhesion strength, we surmised that by spraying or brushing a solid patch with a liquid formulation just before contact with tissue could ensure quick and efficient adhesion of patch to the tissue surface. To test this hypothesis, another lap shear test was performed where the patch was sprayed with precursor solution immediately prior to forming the lap joints. As anticipated, a high adhesion strength was observed within 2 min incubation in PBS and was comparable with the spray mode of application (see FIG. 3C).

### Example 13. Sealing an Injured Lung or Stomach Tissue with the Solid Polymer Patch Made from a Solution of the α-Lipoic Acid (L1) Monomer in Absolute Ethanol Added to a Solution of The NHS-Derivatized α-Lipoic Acid (L2) in DMSO

The combined solution of the α-lipoic acid (L1) monomer in absolute ethanol added to a solution of the NHS-derivatized α-lipoic acid (L2) in DMSO was added to PBS, dried, and formed as a patch as described above. The patch was tested with and without the addition of a liquid adhesive spray. An air-filled porcine lung, and a fluid-filled porcine stomach, were effectively sealed with the solid polymer patch when spraying the solid polymer patch with a liquid form of the adhesive before use, the spray being a liquid form of the adhesive that was made by combining the α-lipoic acid (L1) monomer in absolute ethanol with a solution of the Ac-Gly-OSu-derivatized α-lipoic acid (L3) in DMSO. The lungs were tested for air leaks with a soap and water spray and none were found.

### Experimental:

A range of ex-vivo sealing experiments with fresh porcine stomach and lung were performed. The solid polymer patch (diameter 15 mm) combined with the spray can successfully form an airtight seal of a 5 mm incision in a damaged lung lobe (see FIG. 3K, 3L). Furthermore, to mimic an injury model, a fresh and highly perforated porcine lung was obtained from a slaughterhouse and repaired with bovine pericardium patch grafted to the lung with the aid of adhesive precursor spray. The spray adhesive shows excellent efficacy in sealing the air leaks within 2 min of application, as verified with soap water spray. Similar experiments were also performed with a liquid-filled stomach and solid adhesive patch obtained from L1 and L2 mixture. The spray (L1, L3 mixture), in combination with the solid adhesive patch, bovine pericardium, and PTFE films, provided leakage-free sealing of the damaged stomach.

### Example 14. Sealing Skin, Intestine, Stomach, and Bovine Tendon Tissue with the Solid Polymer Patch Made from a Solution of the α-Lipoic Acid (L1) Monomer in Absolute Ethanol Added To a Solution of the NHS-Derivatized α-Lipoic Acid (L2) in DMSO

The combined solution of the α-lipoic acid (L1) monomer in absolute ethanol added to a solution of the NHS-derivatized α-lipoic acid (L2) in DMSO was added to PBS, dried, and formed as a patch as described above. The patch was tested with and without the addition of a liquid adhesive spray. Burst pressure was tested at 2 minutes and 24 hours incubation time in PBS, and the data shows that the patch, with or without the addition of liquid adhesive before application, provides a rapid and effective tissue adhesion and sealing without burst.

### Experimental:

The burst strength of the solid adhesive patch (L1 and L2) with and without spray was investigated on wet porcine skin, small intestine, and stomach. The pristine patch exhibits 123±32 mmHg burst pressure on wet porcine skin after 2 min of pressing time, increasing to 195±12 mmHg after 24 h incubation in PBS at 37° C. (see FIG. 3F). On the other hand, the patch with spray shows 184±10 mmHg and 237±20 mmHg burst pressure on wet porcine skin, 146±4 mmHg and 180±9 mmHg burst pressure on the porcine small intestine, 252±43 mmHg and 268±45 mmHg burst pressure on the porcine stomach after 2 min and 24 h incubation in PBS respectively (see FIG. 3F).

Augmenting the solid-adhesive patch with the spray led to burst pressures in the range of 150-250 mmHg depending on tissue type, as shown in FIG. 3D, exceeding the hydrodynamic burst strength needed for most in-vivo applications. An ex-vivo experiment with freshly harvested bovine tendon and the solid-adhesive patch with the spray combination showed strong adhesion to the tendon (see FIGS. 3MA-3ME). FIG. 3MA shows a completely dissected bovine tendon. FIG. 3MB shows a wrapping of the transected tendon with a single layer of 2 cm wide patch sprayed ethanolic precursor. FIG. 3MC is a photograph of the wrapped transected tendon loaded in the instrument, before tensile extension at a crosshead speed of 50 mm/min after 5 min incubation in 1× PBS at 37° C. FIG. 3MD is a photograph taken in the middle of the tensile extension and shows elongation of the polymer patch without any interfacial failure. FIG. 3ME is a photograph taken at the end of the tensile extension and shows cohesive rupture (indicated by the arrow) of the patch between two tendon segments, while the patch remains attached to tendon segments. Cohesive rupture of the patch was observed under tensile extension, while the adhesive remains attached to the tendon.

Altogether, these data establish the excellent potential of the newly developed adhesive to provide rapid and effective tissue adhesion and sealing.

### Example 15. Block Undesired Adhesion from Targeted Areas of a Solid Adhesive Patch

This example shows that portions of the polymer patch can be coated with PEG-polymer derivatized with a disulfide monomer to block undesired adhesion from targeted areas of the patch. A telechelic PEG polymer was derivatized with α-lipoic acid monomer and sprayed on an otherwise adhesive surface of a solid polymer patch made from a solution of the α-lipoic acid (L1) monomer in absolute ethanol added to a solution of the NHS-derivatized α-lipoic acid (L2) in DMSO. After spraying on the PEG polymer, a lap joint was formed with the solid patch and incubated in PBS at 37° C. Effective and targeted shielding of the adhesive occurred with use of the PEG layer at both 2 minutes and 24 hours.

### Experimental:

A telechelic PEG polymer was functionalized with α-lipoic acid and sprayed on top of a newly formed adhesive layer before forming the lap joints and incubating in PBS at 37° C. Interestingly, lap shear measurement after 2 min and 24 hours incubation shows significantly lower shear strength, indicating efficient shielding of the adhesive layer and successful incorporation of the non-adhesive PEG layer (see FIG. 3G).

It was demonstrated that the dynamic property of the polymer could be exploited to prepare a non-adhesive free surface to avoid undesired tissue bonding to the free surface of the adhesive. The application of a lipoic-acid-functionalized, antifouling polymer, on top of an in-situ formed poly(α-lipoic acid) adhesive could be used to prevent unwanted biofouling or post-surgical adhesion formation. In some embodiments, medical device coatings can be prepared in this manner.

### Example 16. Testing Biocompatibility of the Adhesives

The following adhesives were biocompatible with NIH 3T3 cells (mouse fibroblast) and human amniotic cells:
1. a solution of the α-lipoic acid (L1) monomer in absolute ethanol added to a solution of the NHS-derivatized α-lipoic acid (L2) in DMSO;
2. α-lipoic acid (L1) monomer in absolute ethanol with a solution of the Gly-OSu-derivatized α-lipoic acid (L3) in DMSO;
3. α-lipoic acid (L1) monomer in a small amount of DMSO, the NHS-derivatized α-lipoic acid monomer (L2), and sodium lipoate (L4) which is a conjugate base of the α-lipoic acid (L1) monomer (L1); and,
4. a solid patch made from a solution of the α-lipoic acid (L1) monomer in absolute ethanol added to a solution of the NHS-derivatized α-lipoic acid (L2) in DMSO, excess phosphate-buffered saline (PBS) was added to create a stable polymer, and the polymer was freeze-dried and compression molded between polydimethylsiloxane (PDMS) coated paper to create the thin film solid adhesive patch.

### Experimental:

In-vitro cytotoxicity studies of (i) the ethanolic precursor solutions of L1 and L2, (ii) the ethanolic precursor solutions of L1 and L3, (iii) the ethanol-free liquid precursor solutions of L1, L2, and L4, and (iv) the solid polymer (L1 and L2 mixture). The tests were performed on NIH 3T3 cells and human amniotic cells. A high density of cells, comparable to growth media controls was observed after a 24 h coculture with the synthesized polymers (see FIG. 4A, 4B, 4C, 4D). Moreover, a Nile red uptake and live dead assay shows excellent biocompatibility of the solid and in-situ polymerized polymers after 24 h incubation. The in-vitro cytotoxicity study of the in-situ polymerized adhesive with human amniotic cells also confirmed the high biocompatibility of each of the adhesives (see FIG. 4A).

### Example 17. Antioxidant Activity of Polymer: Testing for Antioxidant Activity of the Adhesives

This example shows that the adhesive solution created from a solution of the α-lipoic acid (L1) monomer in absolute ethanol added to a solution of the NHS-derivatized α-lipoic acid (L2) in DMSO has antioxidant activity. Macrophages were treated with lipopolysaccharide (LPS) in an LPS-induced oxidative stress model, and the macrophages generated reactive oxygen species (ROS) for 1 hour. The ROS formation was completely suppressed, however, when the LPS treatment was combined with the adhesive solution generating polymer in situ with the treated macrophages. This shows that the adhesives have excellent antioxidant activity and efficacy at neutralizing oxidative stress in cells.

### Experimental:

The antioxidant property of the in-situ synthesized polymer from L1 and L2 mixture was investigated using macrophages and an LPS-induced oxidative stress model. The generation of reactive oxygen species (ROS) was observed after the macrophages were treated with LPS for 1 hour (see FIG. 4E). However, the ROS formation was completely suppressed when the macrophages were treated with LPS together with in-situ synthesized polymer (see FIG. 4F). This result indicates the excellent efficacy of the polymer in neutralizing the oxidative stress in cells.

### Example 18. Antibacterial Activity of Polymer: Testing the Adhesives as Bacterial Barriers that can Help Prevent Bacterial Infections

This example tests the following two adhesives with Escherichia coli (E. coli):
1. a solid patch made from a solution of the α-lipoic acid (L1) monomer in absolute ethanol added to a solution of the NHS-derivatized α-lipoic acid (L2) in DMSO, excess phosphate-buffered saline (PBS) was added to create a stable polymer, and the polymer was freeze-dried and compression molded between polydimethylsiloxane (PDMS) coated paper to create the thin film solid adhesive patch; and,
2. α-lipoic acid (L1) monomer in absolute ethanol with a solution of the Gly-OSu-derivatized α-lipoic acid (L3) in DMSO.

Adhesive 1 was co-cultured with E. Coli, and adhesive 2 formed a polymer in situ that was co-cultured with E. Coli. Both of the adhesives showed a significantly lower density of bacteria on the adhesives in their co-cultures than a control culture plate over 24 hours. This shows that the adhesives have excellent barrier efficacy with E. Coli and would provide a very desirable antibacterial feature to a wound dressing.

### Experimental:

Bacterial infection is one of the major concerns after any surgical intervention or traumatic injuries, and is especially critical for external wound closure to prevent infection into a wound, and for certain internal applications like intestinal perforations and anastomoses, where the repair site must constitute an effective bacterial barrier. Such infections can be avoided by tissue adhesives with bacterial barrier properties. In this context, sulfur is historically known for its excellent antibacterial properties. Hence, we investigated the bacteria barrier properties of the synthesized adhesives with Escherichia coli. Interestingly, the coculture of E. Coli with solid polymer patch synthesized with L1, L2 mixture and in-situ obtained polymer from ethanolic precursor solution of L1 and L3 exhibits a significantly lower density of bacteria on the polymer compared to the control culture plates (see FIG. 4G, 4H, 4I, 4J). Furthermore, a bacteria barrier study of the polymer shows excellent barrier efficacy after 72 h coculture with E. Coli, establishing the potential benefit of the adhesive for wound dressing.

### Example 19. Testing the Adhesives for Undesirable Swelling

This example tests the swelling of adhesives. Swelling of tissue adhesives can create problems in some procedures, and the swelling can reduce adhesive strength.

This example tests the following two adhesives for mass swelling ratio in an in vitro environment, (mₛ-m_{d})/m_{d}, where mₛ is the swollen mass and ma is the dry mass:
1. a solid patch made from a solution of the α-lipoic acid (L1) monomer in absolute ethanol added to a solution of the NHS-derivatized α-lipoic acid (L2) in DMSO, excess phosphate-buffered saline (PBS) was added to create a stable polymer, and the polymer was freeze-dried and compression molded between polydimethylsiloxane (PDMS) coated paper to create the thin film solid adhesive patch; and,
2. α-lipoic acid (L1) monomer in absolute ethanol with a solution of the Gly-OSu derivatized α-lipoic acid (L3) in DMSO.

### Experimental:

Adhesive 1 was incubated in PBS at 37° C. for 24 hrs. The mass of the wet solid patch of Adhesive 1 was recorded before and after freeze-drying and had a swelling ratio of 1.1. Adhesive 2 was polymerized in situ in excess PBS and also incubated in the PBS at 37° C. for 24 hrs. The mass of the wet polymer of Adhesive 2 was recorded before and after freeze-drying and had a swelling ratio of 0.3. This is quite favorable over hydrogel adhesives for biomedical applications, as many hydrogel medical adhesives, unfortunately, swell by several hundred percent.

### Example 20. Testing the Adhesives for Desirable Biodegradation

Biodegradation of the tissue adhesives is crucial for internal medical applications, and it's desired that toxic degradation products are avoided as thy result in acute inflammation and toxicity. This example is an in vitro study of the biodegradation of the polymers in, or produced by, the adhesives.

### Experimental:

Glutathione (GSH)-mediated degradation was used as the model to degrade the poly(α-lipoic acid) polymers, in which the thiolate of the GSH was expected to react with the disulfide bond in the polymer backbone and generate an active thiol group which initiates depolymerization of the polymer. High pressure liquid chromatography was used to verify that the α-lipoic acid monomers (L1) were reformed as desired.

### Example 21. Making and Testing a Pressure Sensitive Adhesive (PSA) System from the α-Lipoic Acid Monomer (L1)

Pressure sensitive adhesives (PSAs) are used in medical applications. This example coats one of the adhesives onto a polyethylene terepthalate (PET) substrate to illustrate use of the adhesives as a pressure sensitive adhesive. Most commonly used PSAs are based on polyacrylates. In addition to being fossil fuel sourced, these polyacrylates have poor recyclability, reusability, and degradability. In sharp contrast, we show that the instant poly(α-lipoic acid) adhesives can potentially be sustainably produced, are biodegradable, and can also be recycled to monomeric feedstock by depolymerization under basic conditions.

### Experimental:

The α-lipoic acid (L1) monomer in absolute ethanol was combined with the Gly-OSu-derivatized α-lipoic acid (L3) to form the adhesive for coating onto the PET substrate, a Hostaphan RN36 PET film using a solution coater with a wet thickness of 200 μm to obtain a final coating thickness of 33 g/m². The coated PET was heated at 100° C. for 4 hrs under vacuum. The heat created a stable, sticky viscoelastic polymer, showing that the stabilizer molecules are also effective at high temperatures. By comparison, for example, pure α-lipoic acid treated under similar conditions also forms a sticky polymer, but within 2-3 days becomes a solid crystalline spot indicating depolymerization of the polymer.

Paper coated with polydimethylsiloxane (PDMS) was used as a release paper for the adhesive. The combined result is a PSA system. The system was stored at ambient conditions for testing.

To test performance as a PSA, industry standard static shear tests were performed on the PSA system against stainless steel (SS), high density polyethylene (HDPE), and polytetrafluoroethylene (PTFE) under dry and wet conditions. Static loads are used because PSAs are expected to perform without rupture for a long time while under static stress. Surprisingly, no shear failure was observed over 7 days when the synthesized PSA tapes were applied on SS and HDPE under dry and wet conditions with a 1 kg hanging weight. A similar static shear test of PSA on PTFE showed stable loading of a 500 g load for over 24 hours in dry and over 7 days in wet conditions. Moreover, a separate shear test showed that the PSA tape could hold over 6 kg of weight when applied on SS under dry and wet conditions. Another underwater experiment showed that a sticky note prepared with our PSA can be repositioned at least three times and can bear a 500 μm static load after each repositioning. Moreover, a freshly positioned sticky note with a contact area of 2.5*3 cm² can hold a 500 μm load over 7 days while submerged under water. These data confirm the attractive adhesion strength of the synthesized PSA under both dry and wet conditions.

The adhesion strength of the synthesized PSA was also evaluated using a 180° peel test. The dry samples for peeling were prepared by adhering the PSA tape to different substrates, then stored for 24 hours at ambient conditions before measurement. The wet samples were assembled and stored underwater for 24 hours before analysis. The PSA tape achieved peel strength of 760+/−127 N/m, 45+/−20 N/m, and 5+/−0.8 N/m against SS, HDPE, and PTFE, respectively, under dry conditions (see FIG. 5A). Underwater, the peel strength was 441+/−21 N/m on SS, 68+/−30 N/m on HDPE, and 6.9+/−2.4 N/m on PTFE (see FIG. 5A). Although a reduction in peel strength was observed in water for SS, an opposite trend was detected for HDPE and PTFE, indicating excellent underwater adhesion performance of the synthesized PSA. Investigation of the failure mechanism showed cohesive failure in SS, whereas adhesive failure was observed in HDPE and PTFE.

To demonstrate recyclability of the PSA adhesive, the PSA tape was treated with 0.5M NaOH for 2 hours, and the backing PET was removed by filtration (see FIG. 5B). Subsequent acidification of the aqueous solution with 6M HCl leads to α-lipoic acid in solid form with an excellent yield (see FIG. 5B). HPLC and ¹H NMR analysis confirmed the high purity of the recycled α-lipoic acid (see FIG. 5C).

### Examples 22-25 are Polymers of Poly(Asparagusic Acid) and Methods of Making the Polymers and Products of the Polymers

The following examples show that the instant technology provides a poly(asparagusic acid), with and without addition of a stabilizer molecule, that has features that are also desirable, including excellent mechanical properties, adhesion strength, cost-effective production, depolymerization into therapeutic monomers, and closed-loop chemical recycling. This is the first report of a poly(asparagusic acid) at all and, as such, it is also the first report of it's desirable characteristics, features, and uses in the medical field and beyond through poly(asparagusic acid) compositions, methods of making, and products. These examples teach that a poly(asparagusic acid) can be provided as (i) a precursor solution of monomer used to form the poly(asparagusic acid); (ii) a solution of poly(asparagusic acid); (iii) a freeze-dried form of the poly(asparagusic acid) that can be reconstituted as a spray in solution or formed into a solid adhesive product; (iv) a combination of a poly(asparagusic acid) and asparagusic acid monomer in solution; or any combination thereof. It should be appreciated that the terms “macromolecular” and “polymeric” can be used interchangeably in some embodiments.

### Example 22. Testing the Stabilizer on a Different Polydisulfide Polymer, Namely Asparagusic Acid

This example is comparing the stabilization of a polymer created from the α-lipoic acid (L1) monomer to a novel polymer created from an asparagusic acid monomer (L8). The asparagusic acid monomer (L8) is added to glycine N-hydroxysuccinimide ester (Gly-OSu) to create a stabilizer molecule (L9), an Gly-OSu-derivatized asparagusic acid monomer. The stabilizer is added to an absolute ethanol solution of the asparagusic acid (L8) that includes a catalytic amount of DMSO for a slow polymerization system that shows a successful creation of a stable polymer with the Gly-OSu-derivatized asparagusic acid monomer.

### Experimental:

An ethanolic solution of a mixture of asparagusic acid (L8) and a stabilizer (L9) was prepared with a mol ratio L8:L3 of 93:7, and a catalytic amount of DMSO was added to ensure the solubility of the NHS esters. The ethanolic adhesive precursor solution prepared in this manner had a total concentration (L8+L3) of 500 mg/ml. The precursor solution was exposed to water to trigger dithiolane polymerization to obtain a stable sticky solid.

### Example 23. Poly(Asparagusic Acid) Will Self-Polymerize in Absolute Ethanol and is More Stable Than Poly(α-Lipoic Acid) in the Absence of a Stabilizer

Although a stabilizer is needed in many applications that require a prolonged stability, the poly(asparagusic acid) without the stabilizer is sufficiently stable for some uses. This example shows that
1. asparagusic acid monomer (L8) will self-polymerize, in the absence of water, in absolute ethanol when concentrated to 600 mg/ml, is set-aside at room temperature for 30 minutes, and the solvent is evaporate under vacuum at 45° C. The poly(asparagusic acid) remained stable for at least 24 hours at room temperature before it is used.
2. the poly(asparagusic acid) can be mixed with pure water to increase polymerization, washed 1× with PBS to mimic human body conditions, and freeze-dried before it is used.
3. the asparagusic acid monomer in pure ethanol can be mixed with acid to speed polymer formation, and the poly(asparagusic acid) can then be mixed with pure water to increase polymerization, washed 1× with PBS to mimic human body conditions, and freeze-dried before it is used.

### Experimental:

Asparagusic acid (500 mg) was dissolved in pure ethanol at a concentration of 600 mg/mL. The clear solution was left aside for 30 min at room temperature, followed by evaporation of the solvent at reduced pressure at 45° C. to obtain a “macromolecular” precursor. The gel-like precursor was stored at room temperature for at least 24 h before use.

Asparagusic acid (500 mg) was dissolved in pure ethanol at a concentration of 600 mg/mL. The clear solution was left aside for 30 min at room temperature, followed by evaporation of the solvent at reduced pressure at 45° C. to obtain a “macromolecular” precursor. The “macromolecular” precursor was further polymerized by adding milliQ water (3 mL), and obtained solid polymer was washed with 1×PBS (pH 7.4). The polymer was freeze-dried to form a dry solid before use.

Asparagusic acid (500 mg) was dissolved in pure ethanol at a concentration of 600 mg/mL, and 5 mol % HCl (4M in dioxane) was added. The clear solution was left aside for 30 min at room temperature, followed by evaporation of the solvent at reduced pressure at 30° C. to obtain a “macromolecular” precursor. The “macromolecular” precursor was further polymerized by adding milliQ water (3 mL), and obtained solid polymer was washed with milliQ water until the pH of the water became 6. Afterward, the solid polymer was washed with excess 1×PBS (pH 7.4). The polymer was freeze-dried to form a dry solid before use.

It was discovered that, although the asparagusic acid will self-polymerize, it is beneficial to the amount and speed of polymerization to add water and acid to the reaction.

### Adhesive Sprays

It was discovered that the above compositions can be formulated as desirable adhesive sprays. Namely, (i) the poly(asparagusic acid) in pure ethanol solution can be diluted 600 mg/ml to 300 mg/ml to form a useful adhesive spray; and (ii) the freeze-dried poly(asparagusic acid), whether or not formed in the acid-catalyzed reaction, can be redissolved into a pure ethanol solution to a concentration of 300 mg/ml to form a useful adhesive spray.

### Solid Adhesive Patches

It was also discovered that the above freeze-dried compositions can be (i) molded, (ii) thermally polymerized, or (iii) solution cast, into desirable solid adhesive patches. Namely, the freeze-dried poly(asparagusic acid), whether or not formed in the acid-catalyzed reaction, can be used to form a solid adhesive patch by (i) compression molding at 75° C. for 15 min between PDMS backing paper to obtain a homogeneous thin film (thickness 250±50 μm); (ii) thermal polymerization by dissolving the freeze-dried polymer in pure ethanol to a concentration of 600 mg/ml, pouring the solution into a PDMS mold, and heating for 3 hours at 80° C. to form the solid adhesive patch; or, (iii) solution cast by dissolving the freeze-dried polymer in pure ethanol to a concentration of 400 mg/mL pouring the solution into a PDMS mold, and evaporating the solution away at ambient temperature to form the solid adhesive patch. The solvent was evaporated at ambient temperature to obtain an adhesive patch. The solid adhesive patches were rested overnight before further use.

Pure ethanol was the solvent of choice for these experiments. However, the skilled artisan will appreciate that any suitable solvent for the poly(asparagusic acid) can be used for the sprays and the patches. Suitable solvents for example, can be selected from the group consisting of ethanol, methanol, water, DMSO, any combination thereof, and the like.

### Example 24. Testing Adhesion Between Biological Tissues Using Poly(Asparagusic Acid) as a Replacement for Medical Grade Cyanoacrylate Superglues

Dry solid poly(asparagusic acid) can be created using the methods taught herein. The freeze-dried solid poly(asparagusic acid), whether or not produced from an acid-catalyzed reaction, can be used to adhere animal tissue together. In this example, the freeze-dried poly(asparagusic acid) from the acid-catalyzed reaction method was dissolved in pure ethanol, an adhesive spray was created, and the spray was used to adhere two pieces of bovine pericardium tissue. After incubation in PBS at 37° C. for 2 minutes and 24 hours, it was discovered that the strength of adhesion was twice as strong after 24 hours.

### Experimental:

The dry solid polymer obtained from acid-catalyzed polymerization of asparagusic acid was dissolved in pure ethanol at a concentration of 300 mg/mL. The liquid precursor was sprayed on two pieces of bovine pericardium tissue surfaces (40 μL in 1 cm²), and the two pieces were immediately brought into contact. After forming the lap joints, they were incubated for 2 minutes in PBS at 37° C. The lap shear strength after 2 minutes on wet bovine pericardium tissue was 121±21 kPa. Interestingly, when the lap joints were incubated for 24 hours in PBS at 37° C., the shear strength increased to 150±8 kPa. A mixed failure (both cohesive and adhesive failure) was observed in 5 repetitions, and adhesive failure were observed after 2 minutes and 24 h of incubation, respectively. A “cohesive failure” means the adhesive stays attached to both sides of the lap joints after breaking the joints, such that the rupture occurs in the adhesive itself, leaving the interface between the substrate and the adhesive intact. An “adhesive failure” means the adhesive stays attached only on one side of the joint after breaking the joints, such that the bond fails between the adhesive and the substrate upon which the adhesive is attached. A “mixed failure” means that the bond breaks in the adhesive itself in some places and where the adhesive bonds to the substrate in other places.

### Example 25. Making a Pressure Sensitive Adhesive (PSA) System from the Asparagusic Acid Monomer (L8)

Pressure sensitive adhesives (PSAs) have many medical uses. This example coats a poly(asparagusic acid) adhesive onto a polyethylene terepthalate (PET) substrate to illustrate use of the adhesive as a pressure sensitive adhesive. Most commonly used PSAs are based on polyacrylates. In addition to being fossil fuel sourced, these polyacrylates have poor recyclability, reusability, and degradability. In sharp contrast, we the instant poly(asparagusic acid) adhesives can potentially be sustainably produced with or without stabilizer for this application, are biodegradable, and can also be recycled to monomeric feedstock by depolymerization under basic conditions.

### Experimental:

Asparagusic acid (500 mg) was dissolved in pure ethanol at a concentration of 600 mg/mL. The clear solution was left aside for 30 min at room temperature, followed by evaporation of the solvent at reduced pressure at 45° C. to obtain a gel-like precursor. No stabilizer was added. The gel-like precursor was stored at room temperature for at least 24 h before use. Afterwards, gel-like precursor was diluted with pure ethanol to obtained a concentration of 300 mg/mL. The obtained homogeneous precursor solution was coated onto HOSTAPHAN RN36 PET films using a solution coater with a wet thickness of 200 μm to obtain a final coating thickness of 15 g/m². Then the monomer mixture-coated PET was heated to 80° C. for 3 hours to obtain PET tape coated with PSA. PDMS release paper was applied on the top of the adhesive layer to protect the adhesive layer as a PSA system temporarily and, finally, the PSA system was stored at ambient conditions for later use.

### Examples 26-35 are Free-Flowing Solids of the Polymers and Methods of Making the Polymers and Products of the Free-Flowing Solids of the Polymers

Above, we taught how the polydisulfide polymers taught herein can be presented in liquid form as precursors to the formation of sticky polymers, either as solutions of monomers, solutions of macromolecules, or combinations thereof. We also showed how to produce spray adhesive products of the liquid forms, and freeze-dried solids of the liquid forms, as well as solid adhesive products that may or may not include a liquid adhesive coating added to the solid adhesive product. The following examples show how to make free-flowing solid products, such as powders, and how to make products directly from the free-flowing form.

### Example 26. Making and Drying a Stabilized Form of Poly(α-Lipoic Acid) in Aqueous Solution from Conjugate Base Monomer Sodium Lipoate (L4), the Stabilizer being the Gly-OSu-Derivatized α-Lipoic Acid (L3)

This example shows that a poly(α-lipoic acid) can be stabilized and dried to create a purified powder of the sticky polymer.

### Experimental:

Sodium lipoate (L4) was dissolved in miliQ at 400 mg/mL, and OSu-derivatized α-lipoic acid (L3) was added with a mol ratio L4:L3 of 90:10. The mixture was stirred overnight before precipitating into ethanol. The solid polymer was freeze-dried to obtain purified “macromolecular” sodium lipoate.

### Example 27. Making a Stabilized Form of Poly(α-Lipoic Acid) in Pure Ethanol Solution with DMSO Using α-Lipoic Acid (L1) as the Monomer, the Stabilizer being the Gly-OSu-Derivatized α-Lipoic Acid (L3)

### Experimental:

A pure ethanol solution of a mixture of α-lipoic acid (L1) and glycine N-hydroxysuccinimide ester of lipoic acid (L3) was prepared at a mol ratio of 93:7, and a catalytic amount of DMSO was added to aid in the polymerization and create an ethanolic adhesive precursor solution with a total monomer concentration of 400 mg/ml and a gelation time of 30 minutes. The solid polymer was dried to obtain purified “macromolecular” poly(α-lipoic acid).

### Example 28. Making a Stabilized Form of Poly(α-Lipoic Acid) from α-Lipoic Acid, Calcium Lipoate, And NHS-Derivatized-α-Lipoic Acid (L2), and Converting the Poly(α-Lipoic Acid) into a Powder Adhesive for Thermal Polymerization into a Solid Adhesive Product

This example shows that ambient moisture and compression temperature and pressure facilitate the formation of the poly(α-lipoic acid) as a homogeneous solid adhesive product.

### Experimental:

A mixture of α-lipoic acid (L1), the conjugate base calcium lipoate, and NHS-derivatized-α-lipoic acid (L2) was prepared at a mol ratio of 75:19:6, was ground together in a porcelain mortar to obtain a homogeneous powder. The powder was used to prepare lap joints and thin film preparation. The thin film was obtained by compression molding the powder mixture at 100° C., 2000 psi for 10 min. Without intending to be bound by any theory or mechanism of action, it is believed that the ambient moisture and compression temperature and pressure facilitate the formation of the poly(α-lipoic acid) as a homogeneous solid adhesive product, which is the solid adhesive patch.

### Example 29. Making a Solid Adhesive Patch of Poly(α-Lipoic Acid) Using a Thermal Polymerization Of α-Lipoic Acid, Sodium Lipoate, and NHS-Derivatized-α-Lipoic Acid (L2)

This example shows that a mixture of dry α-lipoic acid (L1), dry conjugate base monomer sodium lipoate (L4), and dry NHS-derivatized-α-lipoic acid (L2) can form a solid adhesive product from thermal polymerization faster, and at a lower temperature, than in the prior example.

### Experimental:

A mixture of dry α-lipoic acid (L1), dry conjugate base monomer sodium lipoate (L4), and dry NHS-derivatized-α-lipoic acid (L2) at a mol ratio of 52:42:6 was ground together to obtain a sticky solid that was compression molded at 55° C. and 2000 psi for 5 min. A homogeneous solid, adhesive product, which is the solid adhesive patch. Notably, this product was polymerized in nearly half the temperature and half the time of the compressed product made in the prior example.

### Example 30. Making a Free-Flowing Powder Adhesive of Poly(α-Lipoic Acid) from a Mixture of Dry α-Lipoic Acid (L1), Dry Conjugate Base Monomer Sodium Lipoate (L4), and Dry NHS-Derivatized-α-Lipoic Acid (L2)

This example shows that a free-flowing powder of monomer, conjugate base of the monomer, and a stabilizer made from the monomer polymerizes quickly in when placed in contact with a wet surface,

### Experimental:

A mixture of dry α-lipoic acid (L1), dry conjugate base monomer sodium lipoate (L4), and dry NHS-derivatized-α-lipoic acid (L2) at a ratio of 45:45:10 were ground separately and together into a homogeneously mixed, free-flowing powder. The free-flowing powder polymerized quickly to poly(α-lipoic acid) when applied to a wet surface.

### Example 31. a Stable Aqueous Solution of Macromolecular Precursor that can be Activated with An Acidic Activator to Induce Fast Polymerization into a Solid Adhesive Composition of Poly(α-Lipoic Acid)

This example shows that a polymerization in aqueous solution can occur faster with an acidic activator. It should be appreciated that water-based precursors may be more easily implemented, in some embodiments, than pure ethanol solutions.

### Experimental:

Macromolecular sodium lipoate (L4) was prepared as described herein and dissolved in milliQ at 200 mg/mL. Separately, 33 mol % citric acid was dissolved in milliQ at a concentration of 400 mg/mL. The macromolecular sodium lipoate (L4) forms a sticky elastic solid once mixed with a citric acid solution as an activator.

In another experiment, macromolecular sodium lipoate (L4) was prepared as described herein and dissolved in milliQ at 200 mg/mL. Separately, 90 mol % ascorbic acid was dissolved in milliQ at a concentration of 400 mg/mL. The macromolecular sodium lipoate (L4) forms a sticky elastic solid once mixed with an ascorbic acid solution as an activator.

### Example 32. a Stable, Dry and Free-Flowing Solid Adhesive Composition Including Mixture of Dry Monomers and Dry Stabilizers, and Including a Dry Acidic Activator

The free-flowing solid polymerizes quickly into a solid adhesive composition of poly(α-lipoic acid) upon contact with a wet surface. It should be appreciated that stable, dry precursors may be more easily implemented, in some embodiments, than solutions.

### Experimental:

A blend of (i) a dry monomeric sodium lipoate (L4) and (ii) a dry NHS-derivatized-α-lipoic acid (L2) were ground into a fine powder at a ratio of 90:10, and then homogeneously mixed with (iii) 33 mol % fine citric acid powder. The result is a dry and free-flowing, stable monomeric precursor that is stored in an air-tight vial for use as a free-flowing powder adhesive. The free-flowing powder polymerized quickly to poly(α-lipoic acid) when applied to a wet surface.

### Example 33. a Stable, Dry and Free-Flowing Solid Hemostat and Adhesive Composition that Includes a Mixture of Dry Monomeric α-Lipoic Acid (L1), and a Dry Stabilizer of α-Lipoic Acid (L2) and Macromolcular Sodium Lipoate (L4)

The free-flowing solid polymerizes quickly into a solid adhesive composition of poly(α-lipoic acid) upon contact with a wet surface. It should be appreciated that stable, dry precursors may be more easily implemented, in some embodiments, than solutions.

### Experimental:

Macromolecular sodium lipoate (L4) was prepared as described herein and ground into fine dry powder. Then, the powder macromolecular sodium lipoate (L4) powder was mixed with dry lipoic acid monomer (L1) and a dry NHS-derivatized-α-lipoic acid (L2) at a mol ratio of (45:45:10) as a free-flowing solid adhesive and stored in a moisture-free environment. The free-flowing solid adhesive polymerized into a sticky solid when exposed to a wet environment. Moreover, over 100 wt % water absorption was observed, indicating that the free-flowing solid will function well as a hemostat in addition to an adhesive.

### Example 34. BIOACTIVITY: Mixing Dry Poly(α-Lipoic Acid) with a Polylysine, which can Add an Antimicrobial Activity, Improve Cell Adherence, and Facilitate Delivery of a Negatively Therapeutic Active Agent by Attaching to the Positive Charges on the Polylysine

In this example, a dry-polylysine, aka poly(ε-lysine), was added to dry poly(sodium lipoate) to form a homogeneous, free-flowing adhesive powder having the properties added by the ε-polylysine.

### Experimental:

Macromolecular sodium lipoate (L4) was prepared as described herein and ground into fine dry powder. Separately, the poly(ε-lysine) was grounded into a fine powder and mixed with the macromolecular sodium lipoate (L4) with a 1:1 weight ratio. The obtained powder mixture forms a sticky polymer when exposed to a wet environment, such as a contact with a wet surface.

In another experiment, the macromolecular sodium lipoate (L4) was again prepared as described herein and dissolved in milliQ at a concentration of 200 mg/mL to form a macromolecular sodium lipoate (L4) solution. Separately, ε-polylysine, aka poly(ε-lysine), was dissolved in milliQ to form a poly(ε-lysine) solution, and the pH of the poly(ε-lysine) solution was adjusted to 7.2 with a 1M NaOH solution. Then, the macromolecular sodium lipoate (L4) solution was mixed with the poly(epsilon-lysine) solution at a 1:1 weight ratio to obtain a sticky elastic solid. The sticky elastic solid was freeze-dried and ground to obtain free-flowing powder for use as a free-flowing powder adhesive having the functionality added by the poly(ε-lysine).

### Example 35. ADHESION OF POWDER: Testing the Adhesive Strength of a Free-Flowing Powder Adhesive Made from a Dry α-Lipoic Acid Monomer (L1), a Dry Calcium Lipoate Monomer, and a Dry NHS-Derivatized-α-Lipoic Acid Monomer (L2)

### Experimental:

20 mg of a mixture of α-lipoic acid monomer (L1), a dry calcium lipoate monomer, and a dry NHS-derivatized-α-lipoic acid monomer (L2) powder was applied as a single strip on one surface of a first aluminum bar for an overlap area of 1×1 cm to create an adhesive-containing strip, and a lap joint was formed by hand-pressing the adhesive-containing strip with an adhesive-free strip on a second aluminum bar. The lap joint was held together with pressure applied from two mini binder clips (available from Office Depot). The lap joint was cured at 100° C. for 10 min and rested 24 hours before being subjected to a lap shear tensile test at a rate of 50 mm/min until failure.

### The Adhesive Strength was Strong and Maintained Between Dry and Wet Conditions.

The lap shear strength of the bond formed between the aluminum bars bonded by the powder adhesive was surprisingly high (10.5±7 MPa). The adhesive strength realized no significant change (10.1±1.9 MPa), even after submerging in water for 24 h.

### The Adhesive Strength was Repeatable on Several Tested Substrates.

This method was repeated on different substrates, including SS (dry 6.1±2.3 MPa; wet 7.6±2.4 MPa), wood (dry 6.1±0.5 MPa; wet 2±0.15 MPa), and PTFE (dry 0.57±0.14 MPa; wet 0.56±0.03 MPa), all of which maintained excellent adhesion strength in both dry and wet conditions.

SELF-HEALING: Any Ruptured Joint could be Easily Re-Established by Rejoining and Reheating, and the Strength was Maintained Over Several Cycles of Rupture, Rejoining, and Reheating.

Moreover, we observed another surprising and unexpected result: lap shear joints were ruptured intentionally and found to be easily rejoined by reheating at 100° C. for 10 min. After the rupture and rejoining, there was no significant reduction in strength from the original measurements, and this was repeated and observed over 5 cycles of rupturing and rejoining. As an alternative to powders, the solid adhesive patches formed as thin films of thermally polymerized structural adhesive were made from the powder mixture and showed comparable adhesion strength to powder in both dry and wet conditions.

### Examples 36-42 Illustrate the Performance of Some Powder, Liquid, and Solid Medicinal Adhesive Compositions, Including Biocompatibility, Biodegradation, and Drug Elution of Those Compositions

The following examples are based on the following materials, instrumentation and methods:

### Materials and Instrumentation

(±) α-Lipoic acid (αLA) was purchased from Sigma Aldrich (BioReagent, ≥99%; T1395) and AFG Bioscience. The αLA from Sigma Aldrich was used without any purification. αLA from AFG Bioscience was only used to prepare S1 (also referred to herein as “L2”, FIG. 1A), S2 (also referred to herein as “L3”, FIG. 1A), and NaLA according to previously reported method. See, for example, Pal, S.; Shin, J.; DeFrates, K. G.; Arslan, M.; Dale, K.; Chen, H.; Ramirez, D.; Messersmith, P. B. Recyclable Surgical, Consumer, and Industrial Adhesives of Poly(α-Lipoic Acid). Science 2024, 385 (6711). https://doi.org/10.1126/science.ado6292.

N-hydroxy succinimide (NHS) and N,N′-dicyclohexylcarbodiimide (DCC) were purchased from Acros Organics and used without further purification. Anhydrous dichloromethane and tetrahydrofuran were purchased from Sigma Aldrich. The rest of the reagents and solvents were purchased from Sigma-Aldrich or Acros Organics and used as received. Deuterated solvents (DMSO-d6, D2O) were purchased from Sigma Aldrich or TCI America, Inc.

All ¹H NMR and ¹³C NMR spectra were recorded on Bruker 500 MHz NMR spectrometers at the College of Chemistry NMR facility at the University of California, Berkeley. Chemical shifts were given in ppm relative to the residual solvent peak (DMSO-d6: 2.50 for 1H; DMSO-d6: 39.52 for 13C; D20: 4.79 for 1H). Differential scanning calorimetry (DSC) analyses of αLA, NaLA, and αLA-PS were performed on a Mettler-Toledo instrument at a heating/cooling rate of 10° C. min-1 under N₂ atmosphere. All mechanical tests (tensile and shear) were performed using an Instron 3345 machine with a 5 kN load cell and Bluehill 3 software. The elastic modulus was calculated from the slope obtained below 21% strain using Microsoft Excel unless mentioned otherwise. The lap shear tensile experiments were performed according to ASTM-2255-05 at a constant crosshead speed of 50 mm/min. The uniaxial tensile experiments were performed according to ASTM-3039M-17 at a constant crosshead speed of 50 mm/min. The shear strength was calculated as the maximum load divided by the overlap area. The statistical significance was determined with the student's T-test. Rheological measurements were performed on an MCR-301 modular compact rheometer from Anton Paar with a parallel plate geometry (8 mm or 25 mm diameter). Dynamic light scattering was performed on a Malvern Zetasizer NanoZS at 25° C. in ethanol-water mixture at a concentration of 0.1 mg/mL. Conductivity studies were performed on a Keithley 2400 Sourcemeter.

### Methods

### Synthesis of NaDPCA

The embodiments provided herein can use either DPCA, NaDPCA (sodium DPCA), or a combination thereof, in a variety of applications. These compounds can act as a as a prolyl-4-hydroxylase inhibitor. In some embodiments, these compounds, alone or in combination, can promote tissue regeneration. The DPCA remains in suspension, whereas the sodium salt, NaDPCA, is a hydrophilic salt with having a greater solubility than DPCA. Rate of drug delivery can be adjusted by changing ratios of DPCA/NaDPCA to increase or decrease solubility of the drug, where less solubility results in slower delivery.

DPCA (CAS No. 331830-20-7) can be purchased from several chemical providers (MedChemExpress Cat No. HY-121172, NJ, USA). NaOH (33.3 mg, 0.83 mmol, 1 equiv.) and DPCA (200 mg, 0.83 mmol, 1 equiv.) were weighed separately and transferred into a glass vial. To this mixture, 4 mL of milliQ was added, then stirred until a clear solution was obtained. The resulting solution was filtered with a PTFE syringe filter and freeze-dried to obtain NaDPCA.

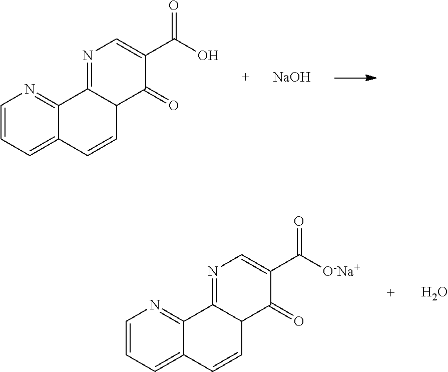

### General Method of Adhesive Preparation for the Following Examples

As described herein, the adhesives can be prepared as powders and liquids for application and adhesion, and each can include a combination of αLA (L1) with NaLA (L4) with S1 (a “stabilizer” also referred to herein as “L2”, FIG. 1A) and/or S2 (a “stabilizer” also referred to herein as “L3”, FIG. 1A):

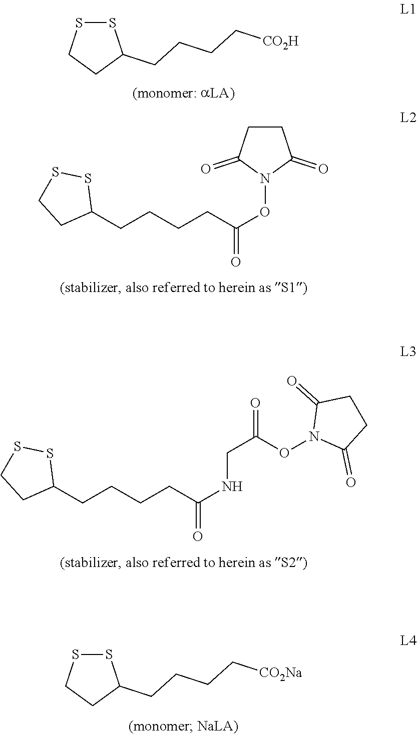

It should be appreciated that αLA (L1) is hydrophobic, so it doesn't absorb enough water at physiologic conditions. However, NaLA (L4) is hygroscopic and takes in water. If a powder adhesive composition is desired, a mixture of αLA and NaLA is preferred, in some embodiments.

### Powder Compositions

### “αLA-PS1” (“PS1” means “powder superglue one” having a 2.21:1 weight ratio, or 2.46:1 mol ratio, of αLA:NaLA; and the ratio of monomer:stabilizer is 16:1)

“αLA-PS1” is used to refer to a powder mixture of αLA (186 mg, 0.91 mmol), NaLA (84 mg, 0.37 mmol), and S2 (“L3”, 30 mg, 0.08 mmol), each separately ground into fine powders. The fine powders were then weighed together and thoroughly mixed in a glass vial. The vial was then stored in sealed conditions at room temperature (~22° C.) until use.

### “αLA-PS2” (“PS2” means “powder superglue two” having a 1:1 weight ratio, or 1.11:1 mol ratio, of αLA:NaLA; and the ratio of monomer:stabilizer is 16:1)

“αLA-PS2” is used to refer to a powder mixture of αLA (135 mg, 0.655 mmol), NaLA (135 mg, 0.592 mmol), and S2 (“L3”, 30 mg, 0.08 mmol), each separately ground into fine powder. The fine powders were then weighed together and thoroughly mixed in a glass vial. The vial was then stored in sealed conditions at room temperature (~22° C.) until use.

Preparing a poly(αLA) patch (polymerize αLA with stabilizer S1 (“L2”)

The αLA (900 mg, 4.37 mmol) and S1 (“L2”, 100 mg, 0.332 mmol) were weighed out separately. The αLA was dissolved in 2.3 mL of absolute ethanol, and S1 was dissolved in 200 μL of DMSO. The two solutions were combined and stirred overnight under sealed conditions until gelled. To this gel, excess 1×PBS was added and vigorously stirred until the gel formed a stiff white solid. This solid was then freeze-dried overnight. The dried solid was compression molded into a thin film on a custom hot press at 75° C. and 2500 psi for 15 min and stored for one day. The patch can be used alone, or it can be “decorated” with additional powder or liquid adhesive in an effort to increase adhesion. To decorate the patch with powder, for example, a thin layer of the αLA-PS1 was applied with finger pressure to the patch in an amount of about 15 mg/cm\hat{ }2. A polydimethylsiloxane (PDMS) release paper was used to protect the adhesive surface, and the combined film was stored at room temperature under sealed conditions.

### Liquid Compositions

### “αLA-LS1” (“LS1” refers to “liquid superglue one” refers to 2.21:1 weight ratio, or 2.46:1 mol ratio, of αLA:NaLA; ratio of monomer:stabilizer is 16:1)

A mixture of αLA (186 mg, 0.91 mmol), NaLA (84 mg, 0.37 mmol), and S1 (“L2”, 30 mg, 0.08 mmol) was weighed together. The solid mixture was dissolved in 300 μL absolute ethanol and 300 μl miliQ water (water amount was varied to prepare different concentration solutions). The resulting solution was heated at 40° C. for one minute, after which ethanol was evaporated under reduced pressure (20 mbar) for 20 minutes at room temperature (22° C.). The obtained solution was transferred to syringes and stored at −20° C. The adhesive was fully thawed under warm running water before further analyses.

### “αLA-LS2” (DRUG LOADED αLA-LS1; “LS2” refers to “liquid superglue two” refers to 2.21:1 weight ratio, or 2.46:1 mol ratio, of αLA:NaLA; ratio of monomer:stabilizer S2 is 16:1, a form of αLA-LS1 loaded with 2.34% NaDCPA)

To prepare drug-loaded liquid adhesive, NaDPCA (6 mg, 0.03 mmol) was weighed with αLA (186 mg, 0.91 mmol), NaLA (84 mg, 0.37 mmol), and S1 (30 mg, 0.08 mmol). Otherwise, the liquid adhesive was prepared as described above for αLA-LS1.

### Example 36. ADHESION OF POWDER: Comparing Tissue Adhesion of Powder Adhesives Over Time with and without Stabilizer

This example tests the adhesive strength of powder adhesive compositions on cow heart tissue. Powder adhesives with and without the presence of the stabilizer are compared. We compare the effects of both stabilizer S1 and stabilizer S2 by making αLA-PS1 with and without stabilizer S1 instead of stabilizer S2. The comparison includes both the αLA-PS1 (“PS1” means “powder superglue one” having a 2.21:1 weight ratio, or 2.46:1 mol ratio, of αLA:NaLA; and the ratio of monomer:stabilizer is 16:1) and the “αLA-PS2” (“PS2” means “powder superglue two” having a 1:1 weight ratio, or 1.11:1 mol ratio, of αLA:NaLA; and the ratio of monomer:stabilizer is 16:1). The drug added is NaDPCA.

### Tissue Preparation

Freshly harvested and untreated bovine pericardium was purchased from Animal Technologies Inc. (Tyler, TX, USA) and stored at −80° C. after quickly freezing it by dipping it in liquid N₂. The frozen tissue was thawed in excess PBS at 37° C. prior to use. One side of a rectangular polycarbonate strip (6×1 cm) was roughened with sandpaper and adhered to the fatty side of the bovine pericardium with Loctite all-purpose liquid superglue and rested for 10 min on a PBS-soaked towel to avoid dehydration of tissue and ensure curing of cyanoacrylate superglue. Afterward, the tissue was trimmed with a scalpel and stored in excess PBS at 37° C. prior to use.

### Preparation of Lap Joints

The solid powder (12.5 mg on either side, 25 mg total) was spread evenly on the tissue surface of two strips (overlap area 1×1 cm), and the lap joint was formed by hand pressing together for 5 seconds. The lap joints were incubated in 1×PBS at 37° C. for 2 min, and a mini binder clip (Office Depot brand) was used as the clamping device. The lap joints were removed from the solution and dried with Kimwipes and subjected to the lap shear tensile test at a rate of 50 mm/min until failure.

For 24-hour incubation, lap joints were prepared and incubated for 2 minutes, as mentioned above. After 2 minutes, the mini binder clips were removed from the lap joints, which were then incubated at 37° C. between PBS-soaked towels for 24 hours. The lap joints were dried with Kimwipes and subjected to the lap shear tensile test at a rate of 50 mm/min until failure.

At least 5 repetitions were performed. The failure mechanism was determined with an N-bromosuccinimide (NBS) base visible assay.

FIGS. 6A-6C illustrate how the adhesive strength of the powder adhesives relies on the addition of stabilizer with the passage of time from 2 minutes to 24 hours, according to some embodiments. The data suggests that regardless of any effect that the ratio of αLA:NaLA may have on adhesion at 2 minutes, there appears to be significantly more adhesion in the presence of stabilizer, regardless of whether the stabilizer is S1 or S2, and the failure appears to be a cohesive failure rather than an adhesive failure.

Without intending to be bound by any theory or mechanism of action, the loss of strength in the absence of stabilizer suggests that the strength loss could be due to cohesive failure in the adhesive rather than adhesion failure between the adhesive and the tissue. FIG. 6C is a photograph of the failure mode of αLA-PS1 on bovine pericardium tissue, with stabilizer S1. The photograph in FIG. 6C further suggests cohesive failure due to the development of yellow on both sides of the lap joint, meaning the failure is not the failure of the adhesion with the tissue but, rather, failure within the polymer that creates the adhesion on the tissue.

### Example 37. Adhesion of Decorated Patch: Comparing Tissue Adhesion of A Poly(αLA) Patch with and without Powder Adhesive Decoration

A poly(αLA) patch adheres by itself on bovine pericardium tissue. This example shows how the adhesive strength of the powder adhesives affects the adhesive strength of a poly(αLA) patch on bovine pericardium tissue. In this example, the how the poly(αLA) patch is “decorated” by the powder form of the adhesive and then applied on the bovine pericardium tissue.

### Tissue Preparation

Freshly harvested and untreated bovine pericardium was purchased from Animal Technologies Inc. (Tyler, TX, USA) and stored at −80° C. after quickly freezing it by dipping it in liquid N₂. The frozen tissue was thawed in excess PBS at 37° C. prior to use. One side of a rectangular polycarbonate strip (6×1 cm) was roughened with sandpaper and adhered to the fatty side of the bovine pericardium with Loctite all-purpose liquid superglue and rested for 10 min on a PBS-soaked towel to avoid dehydration of tissue and ensure curing of cyanoacrylate superglue. Afterward, the tissue was trimmed with a scalpel and stored in excess PBS at 37° C. prior to use.

### Preparation of Lap Joints

One side of a rectangular polycarbonate strip (6×1 cm) was roughened with sandpaper and adhered to the bare patch side (1×1 cm) with Loctite all-purpose liquid superglue. The patch/patch+αLA-PS1 and the lap joint with tissue were hand-pressed together for 5 seconds. The lap joints were incubated in 1×PBS at 37° C. for 2 min, and a mini binder clip (Office Depot brand) was used as the clamping device. The lap joints were removed from the solution and dried with Kimwipes and subjected to the lap shear tensile test at a rate of 50 mm/min until failure.

After 2 minutes, the mini binder clips were removed from the lap joints, which were then incubated at 37° C. between PBS-soaked towels for 24 hours. The lap joints were dried with Kimwipes and subjected to the lap shear tensile test at a rate of 50 mm/min until failure.

FIGS. 7A-7B illustrate how the adhesive strength of the powder adhesives affect the adhesive strength of a poly(αLA) patch on bovine pericardium tissue, according to some embodiments. Decorating the patch increases adhesive strength appreciably.

FIG. 7A shows that the adhesive strength of the patch is 2-3× higher than the patch alone, and this occurs in just 2 minutes. Consistent with FIGS. 6A and 6B, the adhesion strength increases even further from 2 minutes to 24 hours. FIG. 7B is a photograph of the failure mode of αLA-PS1 on a poly(αLA) patch on bovine pericardium tissue. The photograph in FIG. 7B, like FIG. 6C, also suggests cohesive failure due to the development of yellow on both sides of the lap joint, meaning the failure is not the failure of the adhesion with the tissue but, rather, failure within the polymer that creates the adhesion on the tissue. N-5. p-value was calculated with Student's t-test in Microsoft Excel (2 array, 2 tails, 2 type). n.s., p>0.05; * p≤0.05, ** p≤0.01

### Example 38. Biocompatible of Powder: Biocompatibility of Powder Adhesives

A feature of the powder adhesives is their biocompatibility, and this example tests the biocompatibility of αLA-PS1, the powder form of the compositions which is created using the methods taught above.

NIH 3T3 cells were seeded at a density of 1×10⁵ cells/mL in 24 well plates at 1 mL/well. Cells were grown overnight in DMEM with 10% FBS and 5% Pen/strep in an incubator at 5% CO₂ and 37° C. Varying amounts of αLA-PS1 powder was weighed into transwells and heated at 37° C. at RH 90% for 5 min. The obtained gel-containing inserts were then added to the wells and 300 μL of media was added to the inner region, and the lower chamber was filled with 700 μL media. Cells were incubated overnight. Transwells were then removed, and media was replaced with serum-free, PrestoBlue-supplemented media. Control wells grown in normal growth media (positive control) or media supplemented with 0.2 g/L SLS (negative control) were also treated with serum-free, PrestoBlue-supplemented media. All wells were cultured for an additional 3 hours, and Absorbance was analyzed on a Spectrometer. The αLA-PS1 was added in concentrations of 2.5 mg/ml, 1.0 mg/ml, 0.5 mg/ml, and the control had no αLA-PS1.

FIG. 8 illustrates that αLA-PS1 is highly biocompatible with NIH 3T3 (murine fibroblasts) cells at concentrations of 2.5 mg/ml, 1.0 mg/ml, and 0.5 mg/ml, according to some embodiments.

### Example 39. Adhesiveness of Liquid: Comparing Tissue Adhesion of Liquid Adhesives Over Time with and without Stabilizer, with and without Adding a Drug, and at Varying Water Concentrations

This example tests the adhesive strength of liquid adhesive compositions on cow heart tissue. Liquid adhesives with and without the presence of the stabilizer are compared. We compare the adhesion with and without stabilizer S1 making αLA-LS1 with and without stabilizer S1 and testing separately. The comparison includes both the αLA-LS1 (“LS1” refers to “liquid superglue one” refers to 2.21:1 weight ratio, or 2.46:1 mol ratio, of αLA:NaLA; ratio of monomer:stabilizer is 16:1) and the “αLA-LS2” (“LS2” refers to “liquid superglue two” refers to 2.21:1 weight ratio, or 2.46:1 mol ratio, of αLA:NaLA; ratio of monomer:stabilizer S2 is 16:1, a form of αLA-LS1 loaded with 2.34% NaDCPA).

### Tissue Preparation

Freshly harvested and untreated bovine pericardium was purchased from Animal Technologies Inc. (Tyler, TX, USA) and stored at −80° C. after quickly freezing it by dipping it in liquid N₂. The frozen tissue was thawed in excess PBS at 37° C. prior to use. One side of a rectangular polycarbonate strip (6×1 cm) was roughened with sandpaper and adhered to the fatty side of the bovine pericardium with Loctite all-purpose liquid superglue and rested for 10 min on a PBS-soaked towel to avoid dehydration of tissue and ensure curing of cyanoacrylate superglue. Afterward, the tissue was trimmed with a scalpel and stored in excess PBS at 37° C. prior to use.

### Preparation of Lap Joints

The precursor liquid (20 μL) was spread evenly on the tissue surface of two strips (overlap area 1×1 cm), and the lap joint was formed by hand pressing together for 5 seconds. The lap joints were incubated in 1×PBS at 37° C. for 2 min, and a mini binder clip (Office Depot brand) was used as the clamping device. The lap joints were removed from the solution, and dried with Kimwipes, and subjected to the lap shear tensile test at a rate of 50 mm/min until failure.

After 2 minutes, the mini binder clips were removed from the lap joints, which were then incubated at 37° C. between PBS-soaked towels for 24 hours. The lap joints were dried with Kimwipes and subjected to the lap shear tensile test at a rate of 50 mm/min until failure.

The failure mechanism was determined with an N-bromosuccinimide (NBS) base visible assay. N=5. p-value was calculated with Student's t-test in Microsoft Excel (2 array, 2 tails, 2 type). n.s., p>0.05; * p≤0.05, ** p≤0.01

FIGS. 9A-9C illustrate how the adhesive strength of the powder adhesives relies on the addition of stabilizer with the passage of time from 2 minutes to 24 hours, how the addition of 2.34% NaDPCA has no effect on adhesion and, in fact, might surprisingly add adhesive strength, and the concentration of water has an appreciable effect that inverts from 2 minutes to 24 hours, according to some embodiments. FIG. 9A suggests that, consistent with the powder formulations, there appears to be significantly more adhesion in the presence of stabilizer.

FIG. 9B is interesting, as it shows the effect of adding drug to the composition αLA-LS1 (with stabilizer S1 as described above), the drug containing counterpart referred to as “αLA-LS2”. Comparing FIGS. 9A and 9B, it can be seen that the addition of the drug, 2.34% NaDPCA, appears to have no effect on the adhesiveness and, in fact, might surprisingly show an added adhesive strength. As described above, the NaDPCA is added as follows: combining a 2.21:1 weight ratio, or 2.46:1 mol ratio, of αLA:NaLA; in which the ratio of monomer:stabilizer S2 is 16:1, and the αLA-LS1 that results is loaded with 2.34% NaDCPA.

FIG. 9C shows that the amount of water in the compositions is a consideration that should be taken into the design of the compositions taught herein. The water concentration has an appreciable effect on the adhesive strength. The αLA-LS1 composition was tested with water concentrations of 66 wt %, 100 wt %, and 133 wt % at 2 minutes and, surprisingly, the effect is inverted at 24 hours. Strength continues to increase, regardless, from 2 minutes to 24 hours, the 66 wt % composition showing the lowest adhesive strength at 2 minutes and, surprisingly, the highest adhesive strength at 24

### Example 40. Biocompatibility of Liquid: Biocompatibility of Liquid Adhesives

A feature of the liquid adhesives is their biocompatibility, and this example tests the biocompatibility of αLA-LS1, the liquid form of the compositions which is created using the methods taught above.

In vivo biocompatibility of αLA-LS1 was tested by subcutaneous implantation in mice. All mice used in experiments were eight-to twelve-week-old female C57BL/6 mice purchased from Jackson Laboratory and Charles River, USA. Mice were housed in the UC Berkeley vivarium in groups of 3-5 in ventilated cages with a 12-hour light/dark cycle. All procedures and experiments were reviewed and approved by the Animal Care and Use Committee of the University of California, Berkeley (AUP #2023-03-16152) and were performed in accordance with national, state, and institutional guidelines.

The mice were anesthetized with isoflurane and administered a 30 μL subcutaneous dose of αLA-LS1 via a 31-gauge insulin syringe in the upper back. This was immediately followed with a 30 μL bolus of isotonic PBS injected at a site within about 5 mm of the implanted material to ensure rapid polymerization.

Three days after implantation, mice were sacrificed. Livers, kidneys, and skin surrounding injection sites were harvested for histological analyses. Tissues were fixed in 4% paraformaldehyde in PBS for 2 days, dehydrated in a series of alcohols, cleared in xylene, and embedded in paraffin. Blocks were sectioned at 8 μm thickness. Sections were stained with Shandon Hematoxylin Gill 2 (Thermo Scientific, Cat: 6765007) and Eosin Y (Sigma, Cat: HT110216).

FIGS. 10A-10D illustrate H&E-stained images of liver tissue (FIGS. 10A and 10B) and kidney tissue (FIGS. 10C and 10D) from mice that received subcutaneous αLA-LS1 impants, according to some embodiments. All organ tissue appear healthy with no evidence of anomalies resulting from polymer implantation. The bar scale represents 100 μm. These figures illustrate that αLA-LS1 is highly biocompatible in mice, according to some embodiments.

### Example 41. Biodegradability: Biodegradation of Adhesives

A feature of the liquid adhesives is their biodegradability, and this example tests the biodegradability of αLA-LS1, the liquid form of the compositions which is created using the methods taught above.

The liquid polymer was fully polymerized in 1×PBS and then freeze-dried to eliminate excess water. The resulting solid was incubated with 0.1 mM GSH in PBS at pH 7.4 at 37° C. at a concentration of 1 mg/mL polymer to PBS (n=3). The pH of the solution was adjusted to 7.4 every other day, and the GSH solution was refreshed every week. The sample was collected at regular intervals for 15 days, washed with milliQ, and freeze-dried to determine the mass loss.

FIG. 11 illustrates the biodegradation of the liquid adhesive, showing that a complete degradation occurred in about two weeks, in some embodiments.

### Example 42. Drug Release: Drug Release Profile of an Adhesive

This example tests the drug release profile of liquid adhesive compositions in vitro. The profile was taken of an αLA-LS1 composition that was formulated with a drug (“LS1” refers to “liquid superglue one” refers to 2.21:1 weight ratio, or 2.46:1 mol ratio, of αLA:NaLA; ratio of monomer:stabilizer is 16:1), where the drug-containing form is “αLA-LS 2” (“LS2” refers to “liquid superglue two” refers to 2.21:1 weight ratio, or 2.46:1 mol ratio, of αLA:NaLA; ratio of monomer:stabilizer S2 is 16:1, a form of αLA-LS1 loaded with 2.34% NaDCPA). The drug release profile was measured over several days.

The drug-loaded liquid precursor was cured in a small amount of 1×PBS (pH 7.4). Then, more 1×PBS (pH 7.4) was added to the cured adhesive to obtain a concentration of 2.5 mg/mL polymer to 1×PBS (n=3). The solution was incubated at 37° C., and the release of NaDPCA over time was determined by UV-Vis spectroscopy.

FIG. 12 illustrates the drug elution curve of a liquid adhesive composition eluting NaDPCA over several days, according to some embodiments. It should be appreciated that the release profile will vary by drug chemistry, whether the drug is attached to the polymer, and how the drug is attached to the polymer, whether or not a bond is labile, how labile, and the like. The release profile appears to be roughly second order, which is a release rate dependent on the square of the drug concentration. A second order release rate, for example, would slow down more rapidly as the concentration of the drug in the polymer decreases. The release rate of the NaDPCA in this example suggests a second order relationship.

To determine how NaDPCA resides in the precursor, a polydispersity analysis was performed. A mixture of αLA and NaLA in the ratio of 70:30 (mass ratio) was weighed together and dissolved in 1:1 absolute ethanol: milliQ water to produce a 5 mg/mL solution. NaDPCA was dissolved in milliQ water to produce a 0.5 mg/ml solution. The solutions were mixed vigorously at a volume ratio of 1:10 NaDPCA: (αLA+NaLA) to produce a concentrated solution that was further diluted in 1:1 absolute ethanol: milliQ water to a concentration of 0.1 mg/mL. Dynamic light scattering was performed at 25° C. to measure particle size and polydispersity.

FIGS. 13A-13C show that the NaDPCA appears to be present as a nanostructure in view of the opacity it creates in a liquid precursor for αLA-LS1, and the dispersion plots obtained using dynamic light scattering analysis. FIG. 13A is a photograph of the liquid precursor αLA-LS1 without drug, FIG. 13B is the liquid precursor αLA-LS1 with drug showing opacity from drug particles, and FIG. 13C is a dispersion plot of a Dynamic light scattering analysis of the opaque NaDPCA, αLA and NaLA (0.1 mg/mL) in 1:1 ethanol-water mixture. Dispersion plots were obtained for NaDPCA/αLA particles at concentration of 0.1 mg/mL. Z_{avg} 137±8 nm; Pdi 0.240±0.017.

The drug release profile for NaDPCA is shown over a 25 hour period, as the drug is released from the αLA-LS1, apparently as nanoparticles. It appears most of the 65-70% of the drug released occurred in the first 12 hours or so, and about 30% of the drug remained in the polymer after the 25 hours. Comparing FIG. 11 to FIG. 12 suggests that, in the case of the NaDPCA, about 30% of the drug remained in about 75% of the original polymer mass after the first day.

We claim:

1. A medical product, comprising:
a composition including
a plurality of substituted 1,2-dithiolane monomers, the substituted 1,2-dithiolane monomers functional to polymerize through a ring-opening reaction to form a polydisulfide polymer having
a first active thiol end and a second active thiol end; and,
a plurality of repeating units having a substituted dithioalkyl structure from the ring opening reaction as follows 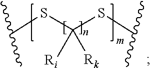
wherein,
n is 3;
each Rᵢ and Rₖ is independently selected from the group consisting of H; alkyl, cycloalkyl, alkenyl, alkynyl, and aryl groups, each of the groups having from 1-8 carbons; hydroxylated alkyl, cycloalkyl, alkenyl, alkynyl, and aryl groups, each of the groups having from 1-8 carbons; and, carboxylated alkyl, cycloalkyl, alkenyl, alkynyl, and aryl groups, each of the groups having from 1-8 carbons; wherein, i and k are integers, and at least one Rᵢ or Rₖ in each repeating unit includes a carbonyl functionality;
and,
m is an integer selected to match a desired molecular weight of the polymer;
a plurality of stabilizer molecules configured to stabilize the first active thiol end of the polymer, the plurality of stabilizer molecules being the substituted cyclic disulfide molecules derivatized to include a functional group that forms a first labile bond with the first active thiol end, the first labile bond selected from the group consisting of a thioester bond, a thiocarbamate bond, and a thioether bond; and,
a bioactive agent;
wherein, the polydisulfide polymer is formed by reacting the plurality of monomers and the plurality of stabilizers in the liquid composition;
adheres to a biological tissue in water;
is biocompatible and biodegradable; and,
releases the bioactive agent for delivery to a target tissue.

2. The medical product of claim 1, the composition further including a plurality of terminator molecules for forming a labile bond with the second active thiol group, the plurality of terminator molecules selected from the group consisting of R′OH, R′CO₂H, and R′SH, and R′ is selected from the group consisting of alkyl, cycloalkyl, alkenyl, cycloalkenyl, alkynyl, cycloalkynyl, and aryl groups, each of the groups having from 1-8 carbons; and, the second labile bond is selected from the group consisting of thioethers, thioesters, and disulfides.

3. The medical product of claim 1, the composition further including a plurality of terminator molecules for forming a labile bond with the second active thiol group, the plurality of terminator molecules selected from the group consisting of alkanols having from 1-8 carbons, alkanoic acids having from 1-8 carbons, and alkylthiols having from 1-8 carbons, and the second labile bond is selected from the group consisting of thioethers, thioesters, and disulfide.

4. The medical product of claim 1, the composition further including a plurality of terminator molecules for forming a labile bond with the second active thiol group, the plurality of terminator molecules selected from the group consisting of ethanol, and a substituted 1,2-dithiolane with a hydroxyl functionality.

5. The medical product of claim 1, wherein:
the repeating unit is a substituted 1,3-dithiopropyl structure as follows 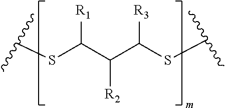
wherein,
m is an integer;
R; includes R₁, R₂, and R₃; and,
each Rₖ is H;
and,
each stabilizer molecule includes the substituted 1,3-dithiopropyl structure.

6. The medical product of claim 5, wherein:
the repeating unit is a substituted 1,3-dithiopropyl structure as follows 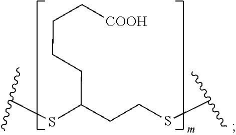
wherein,
m is an integer;
R₁ is a pentanoic acid group, and R₂ and R₅ are each H;
and,
each stabilizer molecule includes the substituted 1,3-dithiopropyl structure, wherein R₁ is a pentanoic acid group, and R₂ and R₃ are each H;
wherein, the bioactive agent is releasably attached chemically to a carboxylate group in at least one of the repeating units;
chemically to a sulfur in at least one of the repeating units;
chemically to the first active thiol end and/or the second active thiol end;
mechanically to the polymer;
or a combination thereof.

7. The medical product of claim 1, wherein the bioactive agent is a wound healing agent.

8. A method of adhering a first biological tissue to a substrate, the method comprising:
applying the medical product of claim 1 to the first biological tissue; and,
creating a joint between the first tissue and the substrate to adhere the first biological tissue to the substrate.

9. The method of claim 8, wherein the substrate is a second biological tissue, and the second biological tissue is selected from the group consisting of a lung tissue, a stomach tissue, a bladder tissue, an intestinal tissue, a vascular tissue, a heart tissue, a dural tissue, an ocular tissue, a cartilage tissue, skin tissue, a fetal membrane tissue, and a tendon tissue.

10. The method of claim 8, wherein the substrate is a medical device selected from the group consisting of wound dressings, collagen membranes, and ostomy devices.

11. The method of claim 8, wherein the method is a wound healing treatment of a subject, the bioactive agent selected to include a wound healing agent.

12. The method of claim 8, wherein the method is a topical tissue closure treatment of a subject, the bioactive agent selected to assist in the topical tissue closure.

13. The method of claim 8, wherein the method is a skin graft treatment of a subject, the bioactive agent selected to assist in the skin graft.

14. The method of claim 8, wherein the method is a wound dressing attachment in a treatment of a subject, the bioactive agent selected to include a wound healing agent.

15. The method of claim 8, wherein the method is a medical device attachment in a treatment of a subject, the bioactive agent selected to assist in the treatment of the subject.

16. The method of claim 8, wherein the method is an internal tissue closure treatment of a subject, the bioactive agent selected to assist in the internal tissue closure.

17. The method of claim 8, wherein the method is an internal tissue closure treatment of a subject, and the internal closure treats an organ puncture, the bioactive agent selected to assist in the internal tissue closure of the organ puncture.

18. The method of claim 8, wherein the method is an internal cartilage repair treatment of a subject, the bioactive agent selected to assist in the internal cartilage repair.

19. The method of claim 8, wherein the method is an internal anastomosis repair treatment of a subject, the bioactive agent selected to assist in the internal anastomosis repair.

20. The method of claim 8, wherein the method is an internal tendon or ligament repair treatment of a subject, the bioactive agent selected to assist in the tendon repair, ligament repair, or a combination thereof.
